SEQUENCE LISTING (128 sequences)

SEQ ID NO: 1 - protein, 15 aa, artificial sequence
  Consensus sequence of antimicrobial peptide
TKFRNSIXXRLKNFN

SEQ ID NO: 2 - protein, 5 aa, ARTIFICIAL
  Consensus sequence of antimicrobial peptide
KRGXG

SEQ ID NO: 3 - protein, 8 aa, artificial
  Consensue sequence of antimicrobial peptide
MVKRGXGE

SEQ ID NO: 4 - protein, 10 aa, artificial
  Consensus sequence for antimicrobial peptide
IXXTLXNFXX

SEQ ID NO: 5 - protein, 15 aa, artificial
  Consensus sequence of antimicrobial peptide
SSRSAAGTIWEFEAF

SEQ ID NO: 6 - protein, 20 aa, artificial
  Consensus sequence of antimicrobial peptide
LXLRGSSRSAAGTIWEFEAF

SEQ ID NO: 7 - protein, 35 aa, artificial
  antimicrobial peptide AGG01
KRGFGKKLRKRLKKFRNSIKKRLKNFNVVIPIPLP

SEQ ID NO: 8 - protein, 35 aa, artificial
  antimicrobial peptide AGG02
KRGLWESLKRKATKLGDDIRNTLRNFKIKFPVPRQ

SEQ ID NO: 9 - protein, 32 aa, artificial
  antimicrobial peptide AGG03
RKKGSKRHKPGSYSVIALGKPGVKKSPYMEAL

SEQ ID NO: 10 - protein, 38 aa, artificial
  antimicrobial peptide A12
MVKRGFGKRLRKRPKKFRNSIKKRLKNFNVVFPVPRPG

SEQ ID NO: 11 - protein, 38 aa, artificial
  Antimicrobial peptide 5
MVKRGLGKNLKRRATKIGNGIKNTLRNFNVVFPIPRPG

SEQ ID NO: 12 - protein, 38 aa, artificial
  Antimicrobial peptide 6
MVKRGLGESLKKRVTKFGDSIRKTLRNFNIIIPIPLPG

SEQ ID NO: 13 - protein, 24 aa, artificial
  Antimicrobial peptide 13
MVKRGFWKSLKRKLKKFGMILGID

SEQ ID NO: 14 - protein, 38 aa, artificial
  Antimicrobial peptide A4
MVKRGFGKNLRRRPTKFRNSIRNRLKNFNIEFPVPRQG

SEQ ID NO: 15 - protein, 38 aa, artificial
  Antimicrobial peptide A5
MVKRGFWKKLKRKAKKFRNSIRKTLKNFNIIIPVPLPG

SEQ ID NO: 16 - protein, 38 aa, artificial
  Antimicrobial peptide A6
MVKRGLGKKLKKKPKKFRNSIKKRLKNFNIEIPIPRQG

SEQ ID NO: 17 - protein, 38 aa, artificial
  Antimicrobial peptide A10
MVKRGFGKKLKKKAKKFRNSIRKRLKNFNVKIPVPLPG

SEQ ID NO: 18 - protein, 38 aa, artificial
  Antimicrobial peptide A11
MVKRGFGKKLRKRATKFRNSIRKRLKNFNIIFPVPRQG

SEQ ID NO: 19 - protein, 24 aa, artificial
  Antimicrobial peptide 10
MVKRGLGERLRRKLTNLGIVLRRH

SEQ ID NO: 20 - protein, 24 aa, artificial
  Antimicrobial peptide 20
MVKRGFGESLRRRVTKLRDDIKKH

SEQ ID NO: 21 - protein, 24 aa, artificial
  Antimicrobial peptide 27
MVKRGFGKRLRRKATKLGMILRRH

SEQ ID NO: 22 - protein, 24 aa, artificial
  Antimicrobial peptide 28
MVKRGLGEKLRRRPKKFGIVLGIH

SEQ ID NO: 23 - protein, 53 aa, artificial
  Antimicrobial peptide B1
MVLGDLLRRVGGKFEEFFGKIRETIRTIRICSHLLLRGSSRSAAGTIWEFEAF

SEQ ID NO: 24 - protein, 64 aa, artificial
  Antimicrobial peptide B6
MVGIVSGEFRGSFEKCVGGIDKSLGILRLSWDLVLRGILRLSWDLVLRGSSRSAAGTIWEFEAF

SEQ ID NO: 25 - protein, 38 aa, artificial
  Antimicrobial peptide E3
MVLGGCLRKGGGRLVGMFGRFRATLKNLLDMLGRRIEG

SEQ ID NO: 26 - protein, 38 aa, artificial
  Antimicrobial peptide F4
MVLGGRSGKVRERFRRIFERYEAKIRTFMEILAAPSQD

SEQ ID NO: 27 - protein, 38 aa, artificial
  Antimicrobial peptide F6
MVLRGFLKRFGGKILRNRGRDGATLKDFLKMIATRIPH

SEQ ID NO: 28 - protein, 29 aa, artificial
  Antimicrobial peptide F7
MVLGGFSRCRWLEEKLRDFMKNLVVRIAG

SEQ ID NO: 29 - protein, 38 aa, artificial
  Antimicrobial peptide F12
MVIGDRFKKFRERLVELRGRNGARLRPLMNIMGGRSAG

SEQ ID NO: 30 - protein, 47 aa, artificial
  Antimicrobial peptide G9
MVLGGFLRNVRWEIWKKGLSLFCQISYFVVRSSSRSAAGTIWEFEAF

SEQ ID NO: 31 - protein, 38 aa, artificial
  Antimicrobial peptide G10
MVIRGLLKRGKGRILGLLGRNGKRLRAIMDMFRRRSQQ

SEQ ID NO: 32 - protein, 32 aa, artificial
  Antimicrobial peptide H6
MVIGDCLRRVRGSLENTVRDLNQGLGILRLRW

SEQ ID NO: 33 - protein, 35 aa, artificial
  Antimicrobial peptide- Retro form of antimicrobial peptide AGG01
PLPIPIVVNFNKLRKKISNRFKKLRKRLKKGFGRK

SEQ ID NO: 34 - protein, 35 aa, artificial
  Antimicrobial peptide-retro form of antimicrobial peptide AGG02
QRPVPFKIKFNRLTNRIDDGLKTAKRKLSEWLGRK

SEQ ID NO: 35 - protein, 32 aa, artificial
  Antimicrobial peptide retro form of antimicrobial peptide AGG03
LAEMYPSKKVGPKGLAIVSYSGPKHRKSGKKR

SEQ ID NO: 36 - protein, 38 aa, artificial
  Antimicrobial peptide retro form of antimicrobial peptide A12
GPRPVPFVVNFNKLRKKISNRFKKPRKRLRKGFGRKVM

SEQ ID NO: 37 - protein, 38 aa, artificial
  Antimicrobial peptide retro form of antimicrobial peptide 5
GPRPIPFVVNFNRLTNKIGNGIKTARRKLNKGLGRKVM

SEQ ID NO: 38 - protein, 38 aa, artificial
  Antimicrobial peptide retro form of antimicrobial peptide 6
GPLPIPIIINFNRLTKRISDGFKTVRKKLSEGLGRKVM

SEQ ID NO: 39 - protein, 24 aa, artificial
  Antimicrobial peptide retro form of antimicrobial peptide 13
DIGLIMGFKKLKRKLSKWFGRKVM

SEQ ID NO: 40 - protein, 38 aa, artificial
  Antimicrobial peptide retro form of antimicrobial peptide A4
GQRPVPFEINFNKLRNRISNRFKTPRRRLNKGFGRKVM

SEQ ID NO: 41 - protein, 38 aa, artificial
  Antimicrobial peptide retro form of antimicrobial peptide A5
GPLPVPIIINFNKLTKRISNRFKKAKRKLKKWFGRKVM

SEQ ID NO: 42 - protein, 38 aa, artificial
  Antimicrobial peptide retro form of antimicrobial peptide A6
GQRPIPIEINFNKLRKKISNRFKKPKKKLKKGLGRKVM

SEQ ID NO: 43 - protein, 38 aa, artificial
  Antimicrobial peptide retro form of antimicrobial peptide A10
GPLPVPIKVNFNKLRKRISNRFKKAKKKLKKGFGRKVM

SEQ ID NO: 44 - protein, 38 aa, artificial
  Antimicrobial peptide retro form of antimicrobial peptide A11
GQRPVPFIINFNKLRKRISNRFKTARKRLKKGFGRKVM

SEQ ID NO: 45 - protein, 24 aa, artificial
  Antimicrobial peptide retro form of antimicrobial peptide 10
HRRLVIGLNTLKRRLREGLGRKVM

SEQ ID NO: 46 - protein, 24 aa, artificial
  Antimicrobial peptide retro form of antimicrobial peptide 20
HKKIDDRLKTVRRRLSEGFGRKVM

SEQ ID NO: 47 - protein, 24 aa, artificial
  Antimicrobial peptide retro form of antimicrobial peptide 27
HRRLIMGLKTAKRRLRKGFGRKVM

SEQ ID NO: 48 - protein, 24 aa, artificial
  Antimicrobial peptide retro form of antimicrobial peptide 28
HIGLVIGFKKPRRRLKEGLGRKVM

SEQ ID NO: 49 - protein, 53 aa, artificial
  Antimicrobial peptide retro form of antimicrobial peptide B1
FAEFEWITGAASRSSGRLLLHSCIRITRITERIKGFFEEFKGGVRRLLDGLVM

SEQ ID NO: 50 - protein, 64 aa, artificial
  Antimicrobial peptide retro form of antimicrobial peptide B6
FAEFEWITGAASRSSGRLVLDWSLRLIGRLVLDWSLRLIGLSKDIGGVCKEFSGRFEGSVIGVM

SEQ ID NO: 51 - protein, 38 aa, artificial
  Antimicrobial peptide retro form of antimicrobial peptide E3
GEIRRGLMDLLNKLTARFRGFMGVLRGGGKRLCGGLVM

SEQ ID NO: 52 - protein, 38 aa, artificial
  Antimicrobial peptide retro form of antimicrobial peptide F4
DQSPAALIEMFTRIKAEYREFIRRFRERVKGSRGGLVM

SEQ ID NO: 53 - protein, 38 aa, artificial
  Antimicrobial peptide retro form of antimicrobial peptide F6
HPIRTAIMKLFDKLTAGDRGRNRLIKGGFRKLFGRLVM

SEQ ID NO: 54 - protein, 29 aa, artificial
  Antimicrobial peptide retro form of antimicrobial peptide F7
GAIRVVLNKMFDRLKEELWRCRSFGGLVM

SEQ ID NO: 55 - protein, 38 aa, artificial
  Antimicrobial peptide retro form of antimicrobial peptide F12
GASRGGMINMLPRLRAGNRGRLEVLRERFKKFRDGIVM

SEQ ID NO: 56 - protein, 47 aa, artificial
  Antimicrobial peptide retro form of antimicrobial peptide G9
FAEFEWITGAASRSSSRVVFYSIQCFLSLGKKWIEWRVNRLFGGLVM

SEQ ID NO: 57 - protein, 38 aa, artificial
  Antimicrobial peptide retro form of antimicrobial peptide G10
QQSRRRFMDMIARLRKGNRGLLGLIRGKGRKLLGRIVM

SEQ ID NO: 58 - protein, 32 aa, artificial
  Antimicrobial peptide retro form of antimicrobial peptide H6
WRLRLIGLGQNLDRVTNELSGRVRRLCDGIVM

SEQ ID NO: 59 - protein, 35 aa, artificial; identical to SEQ ID NO: 33
  Retro-inverted peptide analog of peptide AGG01 wherein each amino acid other than glycine is a D-amino acid

SEQ ID NO: 60 - protein, 35 aa, artificial; identical to SEQ ID NO: 34
  Retro-inverted peptide analog of peptide AGG02 wherein each amino acid other than glycine is a D-amino acid

SEQ ID NO: 61 - protein, 38 aa, artificial; identical to SEQ ID NO: 36
  retro-inverso form of antimicrobial peptide A12 in which all amino acids other than glycine are D amino acids

SEQ ID NO: 62 - protein, 38 aa, artificial; identical to SEQ ID NO: 37
  retro-inverso form of antimicrobial peptide 5 in which all amino acids other than glycine are D amino acids

SEQ ID NO: 63 - protein, 38 aa, artificial; identical to SEQ ID NO: 38
  retro-inverso form of antimicrobial peptide 6 in which all amino acids other than glycine are D amino acids

SEQ ID NO: 64 - protein, 24 aa, artificial
  retro-inverso form of antimicrobial peptide 13 in which all amino acids other than glycine are D amino acids
DIGLIMGFKKLKRKLSKWFGRKVM

SEQ ID NO: 65 - protein, 38 aa, artificial; identical to SEQ ID NO: 40
  retro-inverso form of antimicrobial peptide A4 in which all amino acids other than glycine are D amino acids

SEQ ID NO: 66 - protein, 38 aa, artificial; identical to SEQ ID NO: 41
  retro-inverso form of antimicrobial peptide A5 in which all amino acids other than glycine are D amino acids

SEQ ID NO: 67 - protein, 38 aa, artificial; identical to SEQ ID NO: 42
  retro-inverso form of antimicrobial peptide A6 in which all amino acids other than glycine are D amino acids

SEQ ID NO: 68 - protein, 38 aa, artificial; identical to SEQ ID NO: 43
  retro-inverso form of antimicrobial peptide A10 in which all amino acids other than glycine are D amino acids

SEQ ID NO: 69 - protein, 38 aa, artificial; identical to SEQ ID NO: 44
  retro-inverso form of antimicrobial peptide A11 in which all amino acids other than glycine are D amino acids

SEQ ID NO: 70 - protein, 24 aa, artificial
  retro-inverso form of antimicrobial peptide 10 in which all amino acids other than glycine are D amino acids
HRRLVIGLNTLKRRLREGLGRKVM

SEQ ID NO: 71 - protein, 24 aa, artificial
  retro-inverso form of antimicrobial peptide 20 in which all amino acids other than glycine are D amino acids
HKKIDDRLKTVRRRLSEGFGRKVM

SEQ ID NO: 72 - protein, 24 aa, artificial
  retro-inverso form of antimicrobial peptide 27 in which all amino acids other than glycine are D amino acids
HRRLIMGLKTAKRRLRKGFGRKVM

SEQ ID NO: 73 - protein, 24 aa, artificial
  retro-inverso form of antimicrobial peptide 28 in which all amino acids other than glycine are D amino acids
HIGLVIGFKKPRRRLKEGLGRKVM

SEQ ID NO: 74 - protein, 53 aa, artificial; identical to SEQ ID NO: 49
  retro-inverso form of antimicrobial peptide B1 in which all amino acids other than glycine are D amino acids

SEQ ID NO: 75 - protein, 64 aa, artificial; identical to SEQ ID NO: 50
  retro-inverso form of antimicrobial peptide B6 in which all amino acids other than glycine are D amino acids

SEQ ID NO: 76 - protein, 38 aa, artificial; identical to SEQ ID NO: 51
  retro-inverso form of antimicrobial peptide E3 in which all amino acids other than glycine are D amino acids

SEQ ID NO: 77 - protein, 38 aa, artificial; identical to SEQ ID NO: 52
  retro-inverso form of antimicrobial peptide F4 in which all amino acids other than glycine are D amino acids

SEQ ID NO: 78 - protein, 38 aa, artificial; identical to SEQ ID NO: 53
  retro-inverso form of antimicrobial peptide F6 in which all amino acids other than glycine are D amino acids

SEQ ID NO: 79 - protein, 29 aa, artificial
  retro-inverso form of antimicrobial peptide F7 in which all amino acids other than glycine are D amino acids
GAIRVVLNKMFDRLKEELWRCRSFGGLVM

SEQ ID NO: 80 - protein, 38 aa, artificial; identical to SEQ ID NO: 55
  retro-inverso form of antimicrobial peptide F12 in which all amino acids other than glycine are D amino acids

SEQ ID NO: 81 - protein, 47 aa, artificial; identical to SEQ ID NO: 56
  retro-inverso form of antimicrobial peptide G9 in which all amino acids other than glycine are D amino acids

SEQ ID NO: 82 - protein, 38 aa, artificial; identical to SEQ ID NO: 57
  retro-inverso form of antimicrobial peptide G10 in which all amino acids other than glycine are D amino acids

SEQ ID NO: 83 - protein, 32 aa, artificial; identical to SEQ ID NO: 58
  retro-inverso form of antimicrobial peptide H6 in which all amino acids other than glycine are D amino acids

SEQ ID NO: 84 - DNA, 108 bp, artificial
  Nucleic acid encoding antimicrobial peptide
aagagaggatttgggaagaagttgaggaagagactgaagaaatttcggaacagcattaagaaaagattaaagaactttaacgttgtaattcctatcccactgccaggg

SEQ ID NO: 85 - DNA, 108 bp, artificial
  Nucleic acid encoding antimicrobial peptide
aagagaggactttgggagagtttgaagaggaaagcgacgaaactcggggatgatattaggaatacattaaggaactttaaaattaaatttcctgtcccccgccagggg

SEQ ID NO: 86 - DNA, 108 bp, artificial
  Nucleic acid encoding antimicrobial peptide
aagcgaggatttgggaagaagctcaggaagagactgaagaaatttaggaatagtattaagaagagattaaagaactttaacgttgtaattccaatcccactgccggga

SEQ ID NO: 87 - DNA, 108 bp, artificial
  Nucleic acid encoding antimicrobial peptide
aagcgaggactttgggagagtctcaagaggaaagcgacgaaacttggggatgatattaggaatacattaaggaactttaaaattaaatttccagtcccacggcaggga

SEQ ID NO: 88 - DNA, 128 bp, artificial
  consensus sequence
ccatggtgaagagaggayttkggragarkctcargargarasygamgaaayttrggratrrtattargaakasattaargaactttaamrttrwawttccartcccackgcmgggatagctcgagtta

SEQ ID NO: 89 - protein, 36 aa, artificial
  misc_feature (4)..(4): The ′Xaa′ at location 4 stands for Leu, or Phe.
KRGXXXXLXXXXXKXXXXIXXXLXNFXXXXPXPXXG

SEQ ID NO: 90 - DNA, 108 bp, artificial; identical to SEQ ID NO: 86
  sequence encoding antimicrobial peptide

SEQ ID NO: 91 - DNA, 108 bp, artificial; identical to SEQ ID NO: 87
  sequence encoding antimicrobial peptide

SEQ ID NO: 92 - DNA, 1723 bp, artificial
  bovine beta-casein promoter
tcgaatccatctctatcaattaatgtaattcaaaattggtgagagacagtcattaggaaattctctgtttattgcacaatatgtaaagcatcttcctgagaaaagggaaatgttgaatgggaaggacatgctttcttttgtattccttttctcagaaatcacacttttttgcctgtggccttggcaaccaaaagctaacacataaagaaaggcatatgaagtagccaaggccttttctagttatatctatgacactgagttcatttcatcatttattttcctgacttcctcctgggccatatgagcagtcttagaatgaatattagctgaataatccaaatgcatagtagatgttgatttgggttttctaagcaatacaagacttctatgacagtgagatgtattaccatccaacacacatctcagcatgatataaatgtaaggtatattgtgaagaaaaattatcaattatgtcaaagtgcttactttagaagatcatctatctgtcccaaagctgtgaatatatatattgaacataattaatagacgaaacaaaccttgtaaaaatgagtagtgtaaaatacaactacatttatgaacatctatcactaaagaggcaaagaaagttgaggactgcttttgtaaatgggctcttattaatgaaaagtacttttgaggtctggcttagactctattgtagtacttatggtaagaccctcctcttgtctgggctttcattttctttcttccttccctcatttgcccttccatgaatactagctgataaacattgactcactataaaagatatgaggccaaacttgagctgtccattttaataaatctgtataaataatatttgttctacagaagtatctctaaataaatgtactttctctcttaaaatccctcaacaaatccccactatctagagaataagattgacattccctggagtcacagcatgctttgtctgccattatctgacccctttctctttctctcttctcacctccatctactcctttttccttgcaatacatgacccagattcactgtttgatttggcttgcatgtgtgtgtgctgagttgtgtctcactcttgtcaaccccatgaatgacagtccaccaggctccactatttccagttaagaatactggagtggattgtgtttcctacttcatttgattaatttagtgactttttaaatttttttccatattcaggaggctattctttccttttagtctatactgtcttcgctcttcaggtctaagctatcatcatgtgcttgttagcttgtttctttctccattatagcataaacactaacaactattcaggttagcatgagattgtgttctttgtgtggcctgtgtatttctggtgtgtattagaatttaccccaagatctcaaagacccaccgaatactaaagagacctcattgtagttacaataatttggggactgggccaaaacttccgtgtgtcccagccaaggtctgtagctactggacaatttaatttcctttatcagattgtgaattattccctttaaaatgctccccagaatttttggggacagaaaaataggaagaattcattttctaatcatgcagatttctaggaattcaaatccactattggttttatttcaaaccacaaaattagcatgccattaaatactatatataaacaaccacaaaatcagatcattatcca

SEQ ID NO: 93 - DNA, 595 bp, artificial
  murine prolactin-inducible mammary gland promoter
ggatccaagtagtagttgagtctcatgctaaatgccaccatgttccatcccttttcccaaggctctcagttatgagtctccatatcaaggggctttcctggactttgtcctatggctaggttggacagacaaatatcacctttgatcctaggatgtgatacatcccctttccacgttctgtatgtgtttaggggtaagcatggagttggctgtagccaactgtgttttccagtcacctcccttgtattgtctctgaagcctcctttgttccaaaagtaggttaaggaaatcctgcttcctggaagcagccctaaaagaaatgaaggtttaccagagccaagtgagaagctgggtcatgtgtggaattatgtgggaagaaaacaatacttggtattgactggatcgaggagatggggggagggtggcaggatggagggaggctggcaggctcagggtttctattttggcataagcatctcttcatcattgtcttcctagagagaaggcccggtgccaggaggccagaggccttcttcatacataaaagcagatgaagtgagcggtgtctgcattacaaggtccaggagcagtcaaa

SEQ ID NO: 94 - DNA, 706 bp, artificial
  Bubalis bubalis alpha-lactalbumin promoter
tatttagtggtattggtggttggggatggggaagctgataacatctcagagggtagctagatactgtcatacacacttttcaagttctccatttttgtgaaatagaaagtctctggatccaagttatatgtgattctcagtctctgtggtcatattctatttctattcctgaccactcaacaaggaaccaagatatcaagggacacttcttttgtttcatgcctgggttgagtgggccagtgtcagctctgatcctgggaccatgacatacgatgatgtacagtcctttcccatattctgtatgtctctaaggggaaggaggagttggccatggaccctttgtgcattttctcattttctgattgcttcacttgtattacccctgaggccccctttgttcctgaagtatgttgggcacatcttgcttcctagaaccaacactaccagaaacaacataaagccaaatgggaaacaggatcatgtttgtaacactgcttgggcaggtaacaatacctagtatggactagagattctggggaggaaaggaaaagtggggtgaaattactgaaggaagctagcaggctcaatgtttctttgttggttttactggcctctctcgtcatcctcttcctggatgtaaggcttgatgccagggcccctgaggctttttccacaaataaaaggaggtgagcagtgtggtgacccc

SEQ ID NO: 95 - DNA, 2410 bp, artificial
  murine whey acidic gene promoter
gaattctttcactgctaaaacagggcgggaggagtccagagccctgccactgggtgcagaacatgaagaccccttgagtggaaaggggttatacagctggacagtggtggcgcacacctttaatcccagcactcgggaggcagaggcagacggatctctgagttcgaggccagcctggtctacagaatgagttccgggacagccagggccacacagagaaactcttgtctcgaaaaaccaaaaaaaaacaaaaaaggaaaggggttacacaacagagactcaggtcacagctacccatcacacacaggatacacatacaaaggtgttcacaggcagatgaggaacgaggagaaggggctcaagcaagggcctaaagtttctttttttttttttcttctttttttttttccctgtggcctagagtttcaagaggctgaggacctaggcatgaaccaagaggggccaaaccacttcaagaagcagggggtagcagcagaatctcactatcagccttgagcacagctgggaaggagatccatggaaacaaccaagaaagagctgaaaggggctggagagatggctcagcagttaagagcactgagtgctcttccgaagtcctagattcaaatcccagcaaccatatggtgggtcacaaccatctgtaatgagatctgatgccctcttctggtgtgtctgaagacagctacagtgtacttatgtataataaataaataaatctttagaaagggagggggggagagagagagagagagagagagagagagagagagagagagagagagagagagagagctggaagagggagatctgggaagtctgctggctttatatgctgaccatatatagtcacctgtgtttacacactgtgctcatcactttgaaatctcagtggtttcttctttgagcctgtgtctgtaagttcaccaggagagtggtacataggcaagaataacagccagtgggcataggacacagagtgcatgggccccagcaagactgtagagagaacagagctctggctcctaagacatagggccttctgggaaactcaagcagccaagcaaccctagccagccctttcctggtggccctccttctgttccagcaaaggcggaaatgggaacaggggtggaagcagagcattggcagagcataggtatgacttagtcttgactaacacaagcatggcagtagcctgacagtggcctaaatgtggggatgactgccttagatggggatgactgccttagatgggcatgactgccttagatggggatgactgccttagatggggatgactgccttagatggaacaacaaacatctatgggcatgctgtggaacactggcccacacacggaactgaaggcactggcaatttccatagggcagttaaacctaaaagcatgctcacactcaacaggctgccggaatctcatgagacacctggaatagacgaatgtagaaacagagcagagagttggttgccaaggtctgggggctcagaggacaagcaagaggcgcggctttcctttggggctggcatgaaaggaaatatcgaggttacagcctgagagggcttcccctgacacttcgtattcaaagaggccatgggcaccagtgaagacaaaggagtatggcctgcaccacaggctggcnctgacagtcagtaagcacacagtcactctgggtcatcccatccccttccttgcaagagaaatcaaggaaatgtcccgagaacaatggggcacagtgccagcaggacatctcttcctgcccatggcaccctttggcacggtatgggcccttctgggaaggtggccttccaaattgctctgcacaggcagctccttttcaatgtatgcccgacactctctacatggagcaagcgcctccacactcttagaagaattttagaaaactccagaaaagcaccaggagaagtcaccctcagatgtagcccggactcgagccttgctcaaaacctcctgtcttgttttctatgtgacctgtacaaatttggagctcagaattgcctttgtctgtgatgggttccaacccaaccactcaaagtgacacttgtcacatttgtcactgatcctatttcttctttttctgctccttcattttctccgctttcataataaacaagtattactttttaagtgggggaaaaaatgaccacccttacaaaggactttttaaaaatggcctccattgtggcccttgttcctggcagcctgggcctgctctctctgtgtggccaagaaggaagtgttgtagcccatctagagctgtgccagcctcttcccccaccccacccccaaagtcttcctcctgtgggtcctttaaatgcatcccagacactcagacagccatcagtcacttgc

SEQ ID NO: 96 - DNA, 1332 bp, artificial
  camel whey acidic gene promoter
tctgaagaggggacattttgtgacctgccaacatgcaaagttaccaaaacatagcaagtcgccatcggccaggacctctagaccccagtcgctaaagctcagtgctggctacccagggaggggcctggactgaggtcctagaactctgctgaggccttgtagggactgagatggtgctacctggggcctggggcctggggcctggggcctggagcggggtgagccagggggaccgtagcagcctgtcaaagtggaagggtgttctgggcatctggaactgcatgcagtccaggctgagggccccagagaagtactgaggggctctgtgtccaaggccaagaagccacaggccaggcagaggagtggggcctggaccaggggtgggcactgaccaccagcacacgcagtcatcccgggcacaccttccttgtccaagccctcagggcaaaaggatcaaggaaattccccggagaaggagggcacctagcctgagtgatcatcctgtccccaccccggtccctacacagggacacaggcggggcccttctgggaataggctttcccagtgtctgccctgcgcagaaacagccccgaccctgaacctgcctgcccctccctttctaagacgcccgacatcctctgcacagagcatacggctcctaagtacaagacgactcgttcttgccgtggaaagttcgggaaaagacaaaaagatactgcaggaggaataaaatgccctccaaggtccccccacacccgggctcctcctccttctcctctcccggcggacggagtgctgatttggacacgtcactccctgtccctgaagggcctctccacgaccactgtgttgtgtcattgcggattctatttcttcctttgtctgcccttaattttttgaatgttcacaataaacatgcattactttcaaagtggaaaaaaatggatccactttatgaggaattcttttttatttaaaaacgtggcccaaggcagtggccgcccagtctggggtcggtccaagctggaagtcttttggtccaactggggcagggccagccactcaccctcccccaccgtgttcttcttcccgcctcctcctttaaaggtgccccagggccacgagccaccatctgtcacctgcctgccacctgccaccatgcgctgtcttgccagctctggcctctggcctctgatctcctctggaggctgctctttgcactggccccagccatctccttgccaggtaagcccaggaggggcatcctgccatccctctgctccaggtccccccaccccccgaaatgctgcccaggcctcacagtttggtga

SEQ ID NO: 97 - DNA, 1167 bp, artificial
  rat whey acidic gene promoter
aggaaagcacactcgacactcgaacggactgcctactgtcagatcccatttacatgagatgcccagaatagacagacgcagaaaccgagcagagaggtagttgccaaggcctgggggctcggggaactagcgagaggctgctggcaggcacaggttttcctttggggctggcctgaaacgaaacatcaaggttacagcctgaaagagcttcccctgggactttgtcttcaaagaggagaggccatgggccacagtgaagacctccggccagtcaaaggagtatgggctgcaccataggctggcgcgacagccagtaaacacacagtcactcactctcgagtcattgcatccccttccttgcaagagaagtcaaggaaatgtcccgagagcaatgggcacagtgcccaacaggacatcccatccgggcccatgacaccgttggcacagcatggggcccttctgagaagtgggctttcaaggttccctgcacaggcaatccttttttgatgtgtaccctgtactctctacaaggagcaagtgcctccacattcttataaaactttttagaaaactccagaaaagcaccaagaaaagaaaccatcctctgatgtgactgtacacatttggagctcggaatttccttttttttttttttttaaagatttttatttatttcatgtatgggagcacactgtcgctatcttcagacacaccagaagagggcatcagatcccactggatcccagatggttgtgagccaccatgtggttgctgggacctgaactcaggacctctggaagagcagtcagtgctcccaaccactgagccatctctccagccctcggaatttcctttgtccgagaaaggggtcccaacccaaccattcaaagtgatatctgtcacatttgttacagatcccatttcttccttctctgctccttaatttttttcgttttggccataaacaagttttaccttttaagtgaaaaaataacgaccacccttacaaaggacttcttaaaaatggactccgaattgtgaaccttgttctggtagcctgggcctgctctctgcatgtgtccaagaggaagtgttttagcccatctacgcctatgcaagcctgcccccctccttccccaaagtcttcctcctgtgggtcctttaaat

SEQ ID NO: 98 - DNA, 195 bp, artificial
  Capra hircus beta-lactalbumin promoter
ggcccagagggggacttcctgcttggccccggatggaagaaggcctcctattgtcctcgtagaggaagccaccccggggcccggggatgagccaagtaggattccgggaacctcgtggctggggcgcggcccgggctggctggctggcacgcctcctgtataaggccccgagcccgctgtctcagccctccactc

SEQ ID NO: 99 - DNA, 4204 bp, artificial
  Ovis aries beta-lactalbumin gene promoter
gtcgacctgcaggtcaacggatctctgtgtctgttttcatgttagtaccacactgttttggtggctgtagctttcagctacagtctgaagtcataaagcctggtacctccagctctgttctctctcaagattgtgttctgctgtttgggtctttagtgtctccacacaatttttagaattgtttgttctagttctgtgaaaaatgatgctggtattttgataaggattgcattgaatctgtaaagctacagatatagtcattgggtagtacagtcactttaacaatattaactcttcacatctgtgagcatgatatattttccccctctatatcatcttcaattcctcctatcagtttctttcattgcagttttctgagtacaggtcttacacctccttggttagagtcattcctcagtattttattcctttgatacaattgtgaatgaggtaattttcttagtttctctttctgatagctcattgttagtgtatatatagaaaagcaacagatttctatgtattaattttgtatcctgcaacagatttctatgtattaattttgtatcctgctactttacggaattcacttattagctttttggtgacatcttgaggattttctgaagaaaatggcatggtatggtaggacaaggtgtcatgtcatctgcaaacagtggcagttttccttcttcccttccaacctggatttctttgatttctttctgtctgagtacgactaggattcccaatactataccgaataaaagtggcaagagtggacatccttgtcttatttttctgaccttagaggaaatgctttcagtttttcaccattaattataatgtttactgtgggcttgtcatatgtggccttcattatatggaggtctattccctctatacccaccttgttgagagtttttatcataaaagtatgttgaattttgtcaaaagtttttcctgcatctattgagatgatttttactcttcaattcattaatgatttttattcttcattttgttaatgatttccattcttcaatttgttaacgtggtatatcacattgattgatttgtggatacctttgtatccctgggataaacctcacttgatcatgagctttcaatgtatttttgaattcactttgctaatattctgttgggtatttttgcatctctattcatcaatgatattggcctaagaaaggttttgtctggttttagtatcagggtgatgctggcctcatagagagagtttagaagcatttcctcctctttgatttttcggaatagtttgagtaggataggtattaactcttctttaaatgtttggggacttccctggtgagccggtggttgagaatccgcctcagggatgtgggtttgatccctggtcagggaaccattaataagatcccacatgctgcaggcaacaagcccccaagctgcaaccactgagctgcaaccgctgcagtgcccacaggccacgaccagagaaagcccacatacagcagggaagacccagcacaaccggaaaaaggagtttggtggaatacagctgtgaagccgtctggtcctggactcctgcttgagggaattttttaaaaattattgattcaatttcattactggtaactggtctgttcatattttctatttcttccgggttcagtcttgggagattgtacatgcctaggaatgtgtccgtttcttctaggttgtccattttattggacatgcatgggagcacacagcaccgaccagcgagactcatgctggcttcctggggccaggctggggccccaagcagcatggcatcctagagtgtgtgaaagcccactgaccctgcccagccccacaatttcattctgagaagtgattccttgcttctgcacttacaggcccaggatctgacctgcttctgaggagcaggggttttggcaggacggggagatgctgagagccgacgggggtccaggtcccctcccaggcccccctgtctggggcagcccttgggaaagattgccccagtctccctcctacagtggtcagtcccagctgccccaggccagagctgctttatttccgtctctctctctggatggtattctctggaagctgaaggttcctgaagttatgaatagctttcgggtgaagggcatggtttgtggtcacggttcacaggaagcttgggagaccctgcagctcagacgtcccgagattggtggcacccagatttcctaagctcgctggggaacagggcgcttgtttctccctggctgacctccctcctccctgcatcacccagttctgaaagcagagcggtgctggggtcacagcctctcgcatctaacgccggtgtccaaaccacccgtgctggtgttcggggggctacctatggggaagggcttctcactgcagtggtgccccccgtcccctctgagatcagaagtcccagtccggacgtcaaacaggccgagctccctccagaggctccagggagggatccttgcccccccgctgctgcctccagctcctggtgccgcacccttgagcctgatcttgtagacgcctcagtctagtctctgcctccgtgttcacacgccttctccccatgtcccctccgtgtccccgttttctctcacaaggacaccggacattagattagcccctgttccagcctcacctgaacagctcacatctgtaaagacctagattccaaacaagattccaacctgaagttcccggtggatgtgagttctggggcgacatccttcaaccccatcacagcttgcagttcatcgcaaaacatggaacctggggtttatcgtaaaacccaggttcttcatgaaacactgagcttcgaggcttgttgcaagaattaaaggtgctaatacagatcagggcaaggactgaagctggctaagcctcctctttccatcacaggaaaggggggcctgggggcggctggaggtctgctcccgtgagtgagctctttcctgctacagtcaccaacagtctctctgggaaggaaaccagaggccagagagcaagccggagctagtttaggagacccctgaacctccacccaagatgctgaccagccagcgggccccctggaaagaccctacagttcaggggggaagaggggctgacccgccaggtccctgctatcaggagacatccccgctatcaggagattcccccaccttgctcccgttcccctatcccaatacgcccaccccacccctgtgatgagcagtttagtcacttagaatgtcaactgaaggcttttgcatcccctttgccagaggcacaaggcacccacagcctgctgggtaccgacgcccatgtggattcagccaggaggcctgtcctgcaccctccctgctcgggccccctctgtgctcagcaacacacccagcaccagcattcccgctgctcctgaggtctgcaggcagctcgctgtagcctgagcggtgtggagggaagtgtcctgggagatttaaaatgtgagaggcgggaggtgggaggttgggccctgtgggcctgcccatcccacgtgcctgcattagccccagtgctgctcagccgtgcccccgccgcaggggtcaggtcactttcccgtcctggggttattatgactcttgtcattgccattgccatttttgctaccctaactgggcagcaggtgcttgcagagccctcgataccgaccaggtcctccctcggagctcgacctgaaccccatgtcacccttgccccagcctgcagagggtgggtgactgcagagatcccttcacccaaggccacggtcacatggtttggaggagctggtgcccaaggcagaggccaccctccaggacacacctgtccccagtgctggctctgacctgtccttgtctaagaggctgaccccggaagtgttcctggcactggcagccagcctggacccagagtccagacacccacctgtgcccccgcttctggggtctaccaggaaccgtctaggcccagaggggacttcctgcttggccttggatggaagaaggcctcctattgtcctcgtagaggaagccaccccggggcctgaggatgagccaagtgggattccgggaaccgcgtggctgggggcccagcccgggctggctggcctgcatgcctcctgtataaggccccaagcctgctgtctcagccctcc

SEQ ID NO: 100 - DNA, 3834 bp, artificial
  Capra hircus beta-lactalbumin gene promoter
gtcaacggatctctgtgtctgttttcatgttagtaccatactgttttggtggctgtagctttgagctatagtctgaagtcataaagcccgatacctccagctctgttcttctttctcaagattgtgttctgctgtttgggtctttagtgtctccacacaatttttagaattgtgtgttctagttctgtgaaaaatgatgctggcattttgataaggattgcattgaatctgtaaagctacagatatagtcattgggtagtacaatcactttaacaatattaactcttcaaatccgtgagcatgatgtattttccccctccatatcatcttcaattccttctatcagtttctttcattgcagttttctgagtataggtcttacacctccttgattagagtcattcctcagtattttattcctttgatacaattgtgaatgagatcattttcttagtttctctttctgatagcccattgttagtgtatagaaaagcaacagagttctatgtattaattttgtatcctgcaacagatttctatgtattaattttgtatcctgctactttactgaatttacttattagctttttggtgacatcttaaggattttcttaagaaaatggcatggtatggtaggacaaggtgtcacgtcatctgcaaacagtggcagttttacttcttcccttccagcctggatttctttgatttctttctgtctgagtactgtgactaggattcccaatactataccgaacaaaagtggcaagagtggacatccttgtcttatttttctgaccttagaggaaatgctttcagtttttcaccattaattataatgtttactgtgggcttgtcatatgtggccttcattatatggaggtctattccctctatacccactttgttgagagtttttatcatgaaagtatgttgaattttgtcaacagtttttcctgcatctattgagatgatttttactcttcaattcattaatgatttttattcttcattttgttaatgatttccattcttcaatgtgttaacgtggtatatcacattgattgatttgtggatatctttgtatccctgggataaacctcacctgatcatgagctttcaatgtatttttgaattcactttgctaatattctgctgggtatttttacatctctattcatcaatgatattgacctaagattttctttctttttttttttgtaaagtttttgtgtggttttagtatcagggtgatgctggcctcatagagagagtttagaagcatttcctcctctttgattttttggaatagtttgagtaggataggtattaactcttctttaaatgtttggggacttccctggtgagccggtggttgagaatccgcctcagggatgtgggtttgatccctggtcagggaaccattaataagctcccacatgctgcagggcaacaagcccccaagctgcaaccactgagctgcaaccgctgcagtgcccacgggccacgaccagagaaagcccacatacagcagggaagacccagcacaacctaaaaaaggagtttggtggaatacagctgtgaagccatctggtcctggactcctgcttgagggaatttttttaaaattattgattcaatttcattactgattgccccagtctccctcccacagtggtcagtcccagctgccccaggccagaggtgctttatttccgtctctctctctggatggtattctttggaagctgaagattcctggaagttatgaatagcttcgccctgaagggcatggtttatggtcacggttcacaggaacttgggagaccctgcagctcagacgtcccgaggttggtggcacccagatttcctaagctcgctggggaagggggcgcttgtttctccctggctgacctccctccgccctgcatcacccagttctgagagcagagcggtgctggggggcacagcctctcgcatctgacgccggtgtccaaaccacccgtgctggtgttcggggggctacctatggggaagggctcctcactgcaggggtgccccccgtcccctctgagatcagaagtcccagtccggacagcgaacaggccaagctccctccagaggctccaggaagggatccttgccccccgccgccgcctccagctcctggtgccgcacccttgagcctgatcttgtagacgcctcagtctagtctctgcctccgtgttcacatgccttctccccatgtcccctccatgtccccgttttctctcacaaggacaccggacagtagattagcccctgttccagcctcacctgaacagctcacatctgtaaagacctagattccaaacaagattccaacctgaagttcctggtggatgtgagttctggggcaacatccttcaaccccatcacagcttgcagttcatcacaaaacatggaacctggggtttatcataaaacctaggttcttcatgaaacactgagcttcgaggcttgttgcaagaattaaaggtgctaatacagatcaaggcaaggactgaagctggccaagcctactctttccatcacaggaaaggggggtctgggggcggctgggggtctgctcccgtgagtgagctcttttctgctacagtcaccaacagtctctccgggaaggaaaccagaggccagagagcaagccagagctagtttaggagacccccgaacctccaaccaagatgctgaccaggccagcgggccccctggaaagaccctacagttcaggggggaagaggggctgacccgccaggtccctgctatcaggagacatccccgctatcaggagattcccccaccttgctcccgttccgctaccccaatacgcccaccccacccctgtgatgagtggtttagccacttagaatgtcaactgaaggcttttgcaccctctttgccagaggcacaaggcacccacagcccgctgggtaccaacgcccatgtggattcagccaggaggcctgtcctgcaccctccctgctcgggccccttctgtactcagcaacacacccagcaccagcattcccactgctcctgaggtctgcaggcagctcgctgtagcctgagcggtgtggagggaagtgtcctgggagacttaaaatgtgggaggtgggagggggggaggttgggccctgtgggcctgcccaccccgtgtgcctgcatggagccccagtgctgctcagccgtgcccccgccgcaggggtcaggtcactttcccgtcctgggggttattatgaccgttgtcattttcattgccatttttgctaccctaactgggcagcaggtgcttgcagagccctcgataccgaccaggtcccccctcggagctccacctgaaccccgtgtcacccttgccccagcctgcagaggatggggtcactgcagagatcccttcacccaaggccacggtcacatggtttggaggagctggtgcccaaggcagaggccaccctctaggacacacctgtccccagtgctggctctgacctgcccttgtctaagaggctgaccccggaagtgttcctggcactggcagccagcctgacccagagtccagacacccacctgtgcccccacttctggggtctaccaggaaccgtctaggcccagagggggacttcctgcttggccccggatggaagaaggcctcctattgtcctcgtagaggaagccaccccggggcccggggatgagccaagtaggattccgggaacctcgtggctgggggcccggcccgggctggctggctggcacgcctcctgtataaggccccgagcccgctgtctcagccctccg

SEQ ID NO: 101 - protein, 21 aa, artificial
  alpha-1 lactalbumin secretory signal
MMSFVSLLLVGILFHATQAVN

SEQ ID NO: 102 - protein, 16 aa, artificial
  alpha S1-casein secretory signal
MKLLILTCIVAVAARL

SEQ ID NO: 103 - protein, 16 aa, artificial
  beta lactoglobulin signal peptide
MKCLLLALALTCGAQA

SEQ ID NO: 104 - protein, 162 aa, Macropus eugenii
MRGLTMQVLLLVLGLLSLMTPLGYAQDQPYQDVLNRFIQEYNTKSESESLFRLSVLNLPSQESNDPTAPQLLKFTIRETVCSKSEHRNPEECDFKKNGLVEECIGTVDLDSSSPSVDISCDGPEKVKRGFGKKLRKRLKKFRNSIKKRLKNFNVVIPIPLPG

SEQ ID NO: 105 - protein, 161 aa, Macropus eugenii
MRDSTMQVLLLVLGLLSLMTSLACAQDQPYQDVLNRFIQEYNTKSESESLFRLSVLNLPPEESNDPAVPLLKFTIRETVCPKTEHRNADECDFKKNGLVKQCIGTIDLDSPNPSVDISCDRPAKVKRGLWESLKRKATKLGDDIRNTLRNFKIKFPVPRQG

SEQ ID NO: 106 - protein, 155 aa, Bos taurus
METPRASLSLGRWSLWLLLLGLALPSASAQALSYREAVLRAVDQLNEQSSEPNIYRLLELDQPPQDDEDPDSPKRVSFRVKETVCSRTTQQPPEQCDFKENGLLKRCEGTVTLDQVRGNFDITCNNHQSIRITKQPWAPPQAARLCRIVVIRVCR

SEQ ID NO: 107 - protein, 176 aa, Bos taurus
METQRASLSLGRCSLWLLLLGLVLPSASAQALSYREAVLRAVDQFNERSSEANLYRLLELDPTPNDDLDPGTRKPVSFRVKETDCPRTSQQPLEQCDFKENGLVKQCVGTVTLDPSNDQFDINCNELQSVRFRPPIRRPPIRPPFYPPFRPPIRPPIFPPIRPPFRPPLGPFPGRR

SEQ ID NO: 108 - protein, 144 aa, Bos taurus
MQTQRASLSLGRWSLWLLLLGLVVPSASAQALSYREAVLRAVDQLNELSSEANLYRLLELDPPPKDNEDLGTRKPVSFTVKETVCPRTIQQPAEQCDFKEKGRVKQCVGTVTLDPSNDQFDLNCNELQSVILPWKWPWWPWRRG

SEQ ID NO: 109 - protein, 159 aa, Bos taurus
METQRASLSLGRWSLWLLLLGLALPSASAQALSYREAVLRAVDQLNEKSSEANLYRLLELDPPPKEDDENPNIPKPVSFRVKETVCPRTSQQSPEQCDFKENGLLKECVGTVTLDQVGSNFDITCAVPQSVGGLRSLGRKILRAWKKYGPIIVPIIRIG

SEQ ID NO: 110 - protein, 158 aa, Bos taurus
METQRASLSLGRWSLWLLLLGLALPSASAQALSYREAVLRAVDQFNERSSEANLYRLLELDPPPKEDDENPNIPKPVSFRVKETVCPRTSQQPAEQCDFKENGLVKQCVGTVTLDAVKGKINVTCEELQSVGRFKRFRKKFKKLFKKLSPVIPLLHLG

SEQ ID NO: 111 - protein, 165 aa, Bos taurus
METQRASFSLGRSSLWLLLLGLVVPSASAQDLSYREAVLRAVDQFNERSSEANLYRLLELDPPPEQDVEHPGARKPVSFTVKETVCPRTTPQPPEQCDFKENGLVKQCVGTVTRYWIRGDFDITCNNIQSAGLFRRLRDSIRRGQQKILEKARRIGERIKDIFRG

SEQ ID NO: 112 - protein, 5 aa, artificial
  enterokinase cleavage signal
DDDDK

SEQ ID NO: 113 - DNA, 15 bp, artificial
  Oligonucleotide for overlap extension designated MC1
aagcgaggayttkgg

SEQ ID NO: 114 - DNA, 23 bp, artificial
  Oligonucleotide for overlap extension designated MC2
ytcytgagactctcccmaartcc

SEQ ID NO: 115 - DNA, 24 bp, artificial
  Oligonucleotide for overlap extension designated MC3
tcargargagactgaagaaayttr

SEQ ID NO: 116 - DNA, 26 bp, artificial
  Oligonucleotide for overlap extension designated MC4
tmttcytaatatcatcccyaartttc

SEQ ID NO: 117 - DNA, 22 bp, artificial
  Oligonucleotide for overlap extension designated MC5
argaakgattaaagaactttaa

SEQ ID NO: 118 - DNA, 24 bp, artificial
  Oligonucleotide for overlap extension designated MC6
aytggaaatttaattttaaagttc

SEQ ID NO: 119 - DNA, 16 bp, artificial
  Oligonucleotide for overlap extension designated MC7
ttccartcccackgcm

SEQ ID NO: 120 - DNA, 12 bp, artificial
  Oligonucleotide for overlap extension designated MC8
ctatccckgcmg

SEQ ID NO: 121 - DNA, 25 bp, artificial
  Oligonucleotide for overlap extension designated MC9
catgccatgcaagcgaggayttkgg

SEQ ID NO: 122 - DNA, 26 bp, artificial
  Oligonucleotide for overlap extension designated MC10
taagagctcctatccckgcmgtggga

SEQ ID NO: 123 - DNA, 19 bp, artificial
  Oligonucleotide for overlap extension designated MC17F
aagagaggatttgggaaga

SEQ ID NO: 124 - DNA, 58 bp, artificial
  Oligonucleotide for overlap extension designated MC18R
taatactattcctaaatttcktcrstytcytcytgagmytcttcccaaatcctctctt

SEQ ID NO: 125 - DNA, 42 bp, artificial
  Oligonucleotide for overlap extension designated MC19F
gaaatttaggaatagtattargaakasattaaagaactttaa

SEQ ID NO: 126 - DNA, 46 bp, artificial
  Oligonucleotide for overlap extension designated MC20R
ctatccckgcmgtgggaytggaawtwyaaykttaaagttctttaat

SEQ ID NO: 127 - DNA, 27 bp, artificial
  Oligonucleotide for overlap extension designated MC9-1
catgccatggtgaagagaggayttkgg

SEQ ID NO: 128 - DNA, 26 bp, artificial
  Oligonucleotide for overlap extension designated MC10-1
taactcgagctatccckgcmgtggga

(19) United States
(12) Patent Application Publication
(10) Pub. No.: US 2011/0209228 A1
(43) Pub. Date: Aug. 25, 2011

(54) METHOD OF TREATMENT USING ANTIMICROBIAL COMPOSITION

(76) Inventors: Benjamin Cocks, Victoria (AU); German Spangenberg, Victoria (AU); Jianghui Wang, Victoria (AU)

(21) Appl. No.: 12/736,143

(22) PCT Filed: Mar. 13, 2009
(86) PCT No.: PCT/AU2009/000301

§ 371 Date: May 6, 2011

(30) Foreign Application Priority Data
Mar. 13, 2008 (AU) 2008901249

(51) Int. Cl. A61K 38/02; C07K 2/00; A61K 31/7088; A61P 31/04; A61P 11/00; A61P 1/00; C12N 15/63; C12N 7/01; A01K 67/00; C12P 21/00; C12N 5/10; A23C 9/12

(52) U.S. Cl. 800/4; 530/300; 514/2.6; 514/44 R; 514/2.4; 435/320.1; 435/235.1; 800/14; 435/325; 426/42

(57) ABSTRACT

The present invention provides peptides and analogs and derivatives thereof having antimicrobial activity at least against Streptococcus uberis for the treatment of a range of infectious disease mastitis, otitis externa, clostridial intestinal disease and respiratory disease.

### RELATED APPLICATIONS

This application claims priority from Australian Patent Application No. 2008901249 filed Mar. 13, 2008, the contents of which are incorporated herein in their entirety.

### FIELD OF THE INVENTION

The present invention relates to antibacterial peptide reagents and methods employing same for the treatment of microbial disease(s), in particular microbial disease(s) mediated in part of whole by one or more bacteria and/or fungi.

### BACKGROUND OF THE INVENTION

Human and animal health are valuable commercial sectors and bacterial and fungal pathogenic infections in humans, livestock and domestic pets represent a high cost to these sectors in terms of lost productivity and existing treatment costs. Many bacterial and fungal pathogens of diseases in humans, livestock animals and domestic pets are also recalcitrant to treatment with existing antibiotics, further exacerbating these adverse consequences of infection.

For example, the economic value of the dairy industry worldwide is significant. For example, the International Dairy Foods Association estimates that sales of cow milk in USA alone in 2006 was USD 23.9 billion. This value will be significantly increased when considered on a worldwide scale, and expanded to include all dairy products, e.g., cheese and butter, and to include sales of products from all major animal producers of dairy products, e.g., goats, sheep, camels and buffalo. The pharmaceutical and biotechnology industries have also developed an interest in dairy mammals as suitable bioreactors for the production of biological agents, particularly peptides and proteins. In this respect, the combination of large daily protein output, post-translational processing capabilities, ease of access to recombinant protein by milking and low capital cost of production plants, i.e., farms compared to high volume industrial fermenters makes dairy mammals excellent candidates for the production of recombinant peptides and proteins (Echelard, Curr. Opin. Biotechnol., 7: 536-540, 1996).

Mastitis is currently the most economically important disease of dairy mammals (Pyörälä Reprod. Dom Anim., 37: 211-216, 2002 and Bergonier et al., 34: 689-716, 2003). The annual costs of mastitis in dairy cattle alone is estimated to be about 10% of the total sales of farm milk, i.e., about USD 2 billion in USA alone (Radostits et al.,: Veterinary Medicine: A Textbook of the Diseases of Cattle, Sheep, Pigs, Goats and Horses, Ninth Ed., Elsevier Health Sciences, 2000). In this respect, the costs of mastitis extend beyond treatment and prevention costs and include losses in milk production, labor costs and loss of animals due to culling Other effects of mastitis include the impact of agricultural use of antibiotics that are used to treat mastitis on the development of antibiotic resistant human pathogens (Smith et al., Proc. Natl. Acad. Sci., USA 99: 6434-6439, 2002) and the effect of residues of these antibiotics on human health (Clement Anim. Pharm., 407: 22-23, 1998). Moreover, the limited milk production resulting from mastitis limits the utility of mammals as bioreactors for producing pharmaceutical agents.

Mastitis is an inflammatory reaction of the mammary gland, usually to microbial infection. This condition is characterized by an influx of somatic cells, primarily polymorphonuclear neutrophils (PMN), into the mammary gland, and by an increase in milk protease content (Verdi et al., J. Dairy Sci., 70: 230-242, 1987). Mastitis is generally classified into clinical infections and non-clinical infections. Clinical infections are diagnosed by red, swollen appearance of a mammary gland and flakes or clots (protein aggregates) in the milk. Sub-clinical infections show no obvious symptoms. The majority of cases of mastitis are caused by one or more of Staphylococcus aureus, Streptococcus dysgalactiae, Streptococcus agalactiae, Streptococcus uberis or Escherichia coli. In this respect, S. aureus, S. dysgalactiae and S. agalactiae have a contagious route of transmission, whereas S. uberis and E. coli are environmental pathogens (Kerr and Wellnitz, J. Anim. Sci., 81: 38-47, 2003).

Susceptibility of a mammal to an intra-mammary infection leading to mastitis dramatically increases during early involution and during the periparturient period (Nickerson J. Dairy Sci., 72: 1665-1678, 1989; and Oliver and Sordillo, J. Dairy Sci., 71: 2584-2606, 1988). These infections are often associated with clinical mastitis during early lactation, and can have a marked detrimental effect on subsequent milk yield and/or quality. Susceptibility to mastitis is also high during the prepartum period in first-lactation bovine heifers (Nickerson et al., J. Dairy Sci., 78: 1607-1618, 1995). These infections are associated with a decrease in alveolar epithelial and luminal area and an increase in connective tissue in the mammary gland, potentially causing a life-long reduction in milk yield in the infected mammal.

The incidence of contagious mastitis has declined over the last thirty years as a result of a five point control plan that recommends use of correctly maintained milking equipment, post-milking teat disinfection, both therapeutic and prophylactic use of antibiotics and culling of persistently infected animals (Bramley and Dodd, J. Dairy Res., 51: 481-512, 1984). Notwithstanding that implementation of this plan has almost eliminated S. dysgalactiae and S. agalactiae from many herds, as discussed supra the use of antibiotics is both expensive and may have a detrimental effect on human health. Moreover, S. aureus, which accounts for 15% to 30% of contagious infections has proven to be resistant to traditional management approaches. In this respect, the cure rate of treatment of S. aureus infection is often less than 15%. This reduced cure rate is attributed in part to antibiotic resistant strains of S. aureus, and to incomplete penetration of the antibiotics through a mammary gland thereby permitting bacteria to survive within the gland. Moreover, S. aureus is able to survive within mammary gland epithelial cells, within which antibiotic concentration is insufficient to cause bacterial cell death (Craven and Anderson J. Dairy Res., 51: 513-523, 1984, and Yancey et al., Eur. J. Clin. Microbiol. Infect. Dis., 10: 107-113, 2991).

Antibiotic treatment is used to treat mastitis caused by environmental pathogens, e.g., S. uberis, however these pathogens are often resistant to conventional antibiotics. Moreover, recurrence of infection from environmental reservoirs, e.g., within dairy barns, is a continuing problem (Kerr and Wellnitz, supra).

Current therapies for mastitis make use of conventional antibiotics, e.g., β-lactams including penicillins and cephalosporins. Notwithstanding that these antibiotics may be effective in the treatment of some pathogens that cause mastitis, some bacterial pathogens are resistant to these compounds. There is also a risk that ongoing use of these compounds may contribute to emergence of antibiotic resistant human pathogens (Smith et al., Proc. Natl. Acad. Sci. USA, 99: 6434-6439, 2002). Moreover, concern that accidental exposure of susceptible consumers of milk products containing traces of the antibiotic may produce drug-induced anaphylaxis has resulted in regulatory-bodies enforcing a post-treatment milk discard period and strict industry surveillance of all milk shipments (Kerr and Wellnitz, supra). Clearly, these additional regulatory requirements lead to increased cost in production of dairy products and loss of commercially valuable resources through wastage.

The disadvantages of conventional antibiotics in the treatment and/or prevention of mastitis has lead to the dairy industry investigating alternative sources of therapeutic and/or prophylactic compounds, e.g., using vaccines and immuno-regulatory agents. For example, researchers have immunized animals with live or attenuated bacteria, cell lysates or subunit vaccines comprising one or more cellular components of a bacterium. However, such approaches have met with limited success, e.g., because of a failure to protect against more than a single causative pathogen.

In a further example, Clostridium difficile is found in many hospital environments and is a major cause of diarrhoea, hemorrhagic enteritis and abomasitis in humans e.g., undergoing antibiotic therapy. In the US, more people die from C. difficile infections than all other intestinal infections combined, with most deaths involving patients aged 65 years or over. The disease is believed to have contributed to more than 8,000 deaths in the UK in 2007. The cost of managing the disease worldwide is high. The chronic bowel infection caused by C. difficile is also very difficult to treat.

In a further example, Clostridium perfringens causes clostridial necrotizing enteritis in humans e.g., subjects receiving TNF antagonist therapy (e.g., Enbrel, Humira) for chronic inflammation (essentially immunocompromised people). The disease comprises necrotizing inflammation of the jejunum and ileum. C. perfringens may also causes other severe inflammatory diseases in the small bowel e.g., the jejunum, wherein inflammation is segmental, involving small or large patches with varying degrees of haemorrhage and necrosis. Perforation of the intestine may also occur in severe cases.

Infections by C. perfringens are also the most common causes of clostridial hemorrhagic enteritis in neonatal ruminants, and in domestic animals such as felines and canines, especially in animals that have been overfed, fed on barely thawed or contaminated colostrum, or in animals that have decreased gut motility. The gram-positive bacilli are often found on affected mucosa and in sub-mucosa. For example, infected calves may exhibit tympany, hemorrhagic abomasitis, and abomasal ulceration. Infected animals develop pasty yellow and bloody diarrhea, and the abdomen becomes distended and painful. Known therapies are not highly effective and the disease has a high mortality rate.

In a further example, Respiratory Disease in livestock animals is a common cause of illness and death in pigs/swine and cattle, e.g., causing between 50% and 90% of all sickness and deaths in Australian feedlot cattle, and the disease is common in the first four weeks after cattle enter the feedlot. In addition to the costs associated with treatment, wasted feed and cattle deaths, Bovine Respiratory Disease (BRD) causes performance losses due to decreased weight gain and feed conversion efficiency of infected animals. During the transition from a paddock to a feedlot system, feedlot cattle are commonly exposed to a range of stress factors that may depress their immune system. Respiratory disease in livestock animals is caused by a plurality of viral and bacterial pathogens. Exemplary bacterial pathogens associated with respiratory disease in cattle include Haemophilus somnus, Pasteurella multocida, Mannheimia (Pasteurella) hemolytica and Mycoplasma spp. e.g., M. bovis. Exemplary bacterial pathogens associated with respiratory disease in swine (Swine Respiratory Disease, SRD) include Actinobacillus pleuropneumoniae, Actinobacillus suis, P. multicoda, Streptococcus suis, Haemophilus parasuis, Bordetella bronchiseptica, Salmonella choleraesuis and Mycoplasma hyopneumoniae. Antimicrobial resistance among bacterial pathogens responsible for BRD and SRD is a serious global problem that complicates the management of infection. For example, resistance to treatment with known antibiotics e.g., tylosin, nalidixic acid, sulfamethozaxole trimethoprim and ampicillin, ranges from 35% to 55%.

In further examples, human and animal inflammatory diseases of the ear such as otitis externa and otitis media caused e.g., by Proteus spp., and/or Pseudomonas spp. and/or the yeast/fungus Malassezia pachydermatis and/or Staphylococcus spp. result in alterations in the normal environment of the canal, including enlargement of the apocrine (cerumen) glands and/or reduced width of the ear canal and/or calcification of auricular cartilage and/or rupture of the tympanum and/or a painful inflammation of the vestibulocochlear nerve. Infections are often mixed with, or due entirely to Malassezia pachydermatis. Infection also results in the production of purulent and malodorous exudate. It is a common disease in humans and domestic animals such as felines and canines, especially pendulous-eared canines and animals with stenosis or hirsutism or the ear canal. Current treatments are costly, and include administration of systemic antibiotics e.g., trimethoprim-potentiated sulfonamides, cephalexin, enrofloxacin and clindamycin, and/or systemic antifungals e.g., ketoconazole. Systemic anti-inflammatory corticosteroids may also be administered e.g., prednisone, to reduce swelling and/or pain associated with otitis externa. Resistance to such antibiotics may occur.

In a further example, the two most common cutaneous fungal infections in small animals are dermatophytosis and Malassezia dermatitis. Malassezia can be found in very large proportion on the skin of diseased animals, especially dogs. It often causes illness together with other pathogens (e.g. Staphylococcus intermedius). Some breeds are predisposed. Malassezia pachydermatis dermatitis is resistant to treatment with glucocorticoids. In cats, disease produced by Malassezia is most often seen as ear infections, severe acne, and generalized redness and scaling. Severe disease may be associated with underlying HIV infection. Dogs with corneal ulcer are more likely to develop Malassezia pachydermatis dermatitis which suggests its possible role at least as an aggravating factor in the disease. Current treatments are limited, including topical application of ketoconazole, and terbinafine has been used to decrease itchiness.

Accordingly, there is a need in the art for suitable alternatives for the treatment (prophylactic and/or therapeutic) of organisms such as those referred to supra that are resistant to conventional antibiotic treatment and/or against which conventional antibiotics are not effective e.g., not bacteriostatic or bactericidal and/or for which treatment regimes are limited and there is a potential for resistance to develop and/or for which existing treatment regimes are otherwise poorly effective.

There is also a need in the art for suitable therapeutic and prophylactic peptide-based and/or protein-based reagents and methods for the treatment of microbial disease(s) such as one or more infections of the mammary gland (e.g., mastitis) and/or one or more infections of the respiratory system (e.g., respiratory disease in humans, and/or livestock such as pigs and/or cattle, and/or domestic animals such as dogs and/or cats) and/or one or more infections of the digestive system (e.g., clostridial intestinal disease(s) in humans, and/or livestock such as pigs and/or cattle, and/or domestic animals such as dogs and/or cats, including diarrhoea and/or hemorrhagic enteritis and/or abomasitis), and/or one or more infections of the ear(s) (e.g., otitis externa or otitis media) and/or one or more infections of the skin (e.g., dermatophytosis or Malassezia dermatitis). In the case of applications in the dairy industry, there is also a desire for such peptide and/or protein-based reagents to have low activity against one or more gut-friendly bacteria e.g., lactobacilli.

There is also a need in the art for suitable therapeutic and prophylactic peptide-based and/or protein-based reagents and methods for the treatment of microbial disease(s) in humans such as one or more infections of the mammary gland (e.g., mastitis) and/or one or more infections of the digestive system (e.g., clostridial intestinal disease(s) such as diarrhoea and/or hemorrhagic enteritis and/or nectrotising enteritis and/or abomasitis), and/or one or more infections of the ear(s) (e.g., otitis externa or otitis media).

There is also a need in the art for suitable therapeutic and prophylactic peptide-based and/or protein-based reagents and methods for the treatment of microbial disease(s) in mammals that produce food products for human consumption such as livestock animals and/or animals that act as bioreactors e.g., pigs and/or cattle and/or poultry, such as one or more infections of the mammary gland (e.g., mastitis) and/or one or more infections of the respiratory system (e.g., bovine respiratory disease and/or swine respiratory disease) and/or one or more infections of the digestive system (e.g., clostridial intestinal disease(s) such as diarrhoea and/or hemorrhagic enteritis and/or abomasitis). In the case of applications in the dairy industry, there is also a desire for such peptide and/or protein-based reagents to have low activity against one or more gut-friendly bacteria e.g., lactobacilli.

There is also a need in the art for suitable therapeutic and prophylactic peptide-based and/or protein-based reagents and methods for the treatment of microbial disease(s) in domesticated mammals such as dogs and/or cats such as one or more infections of the respiratory system (e.g., feline respiratory disease) and/or one or more infections of the ear(s) (e.g., otitis externa or otitis media) or one or more infections of the skin (e.g., dermatophytosis or Malassezia dermatitis).

There is also a need in the art for suitable therapeutic and prophylactic peptide-based and/or protein-based reagents and methods for the treatment of microbial disease(s) mediated by Streptococcus spp. e.g., S. uberis and/or S. suis and/or S. agalactiae.

There is also a need in the art for suitable therapeutic and prophylactic peptide-based and/or protein-based reagents and methods for the treatment of microbial disease(s) mediated by enterobacteria e.g., Escherichia coli and/or Clostridium difficile and/or Clostridium perfringens.

There is also a need in the art for suitable therapeutic and prophylactic peptide-based and/or protein-based reagents and methods for the treatment of microbial disease(s) mediated by Staphylococcus spp. e.g., S. aureus and/or S. schleifen subsp. coagulans and/or S. schleiferi and/or S. intermedius and/or S. epidermis and/or S. pseudointermedin.

There is also a need in the art for suitable therapeutic and prophylactic peptide-based and/or protein-based reagents and methods for the treatment of microbial disease(s) mediated by Pasteurella spp. e.g., Mannheimia haemolytica (P. haemolytica) and/or P. multocida.

There is also a need in the art for suitable therapeutic and prophylactic peptide-based and/or protein-based reagents and methods for the treatment of microbial disease(s) mediated by Actinobacillus spp. e.g., A. pleuropneumoniae (APP) and/or A. suis

There is also a need in the art for suitable therapeutic and prophylactic peptide-based and/or protein-based reagents and methods for the treatment of microbial disease(s) mediated by Salmonella choleraesuis.

There is also a need in the art for suitable therapeutic and prophylactic peptide-based and/or protein-based reagents and methods for the treatment of microbial disease(s) mediated by Mycoplasma spp. e.g., Mycoplasma hyopneumoniae.

There is also a need in the art for suitable therapeutic and prophylactic peptide-based and/or protein-based reagents and methods for the treatment of microbial disease(s) mediated by Proteus spp. e.g., Proteus mirabili.

There is also a need in the art for suitable therapeutic and prophylactic peptide-based and/or protein-based reagents and methods for the treatment of microbial disease(s) mediated by Haemophilus spp. e.g., H. parasuis and/or or H. somnus.

There is also a need in the art for suitable therapeutic and prophylactic peptide-based and/or protein-based reagents and methods for the treatment of microbial disease(s) mediated by Pseudomonas spp.

There is also a need in the art for suitable therapeutic and prophylactic peptide-based and/or protein-based reagents and methods for the treatment of microbial disease(s) mediated by Malassezia pachydermatis.

There is also a need in the art for suitable therapeutic and prophylactic peptide-based and/or protein-based reagents and methods for the treatment of microbial disease(s) mediated by Bordetella spp. e.g., B. bronchiseptica.

The following publications provide conventional techniques of molecular biology. Such procedures are described, for example, in the following texts that are incorporated by reference:
1. Sambrook, Fritsch & Maniatis, Molecular Cloning: A Laboratory Manual, Cold Spring Harbor Laboratories, New York, Third Edition (2001), whole of Vols I, II, and III;
2. DNA Cloning: A Practical Approach, Vols. I and II (D. N. Glover, ed., 1985), IRL Press, Oxford, whole of text;
3. Ausubel et al., Current Protocols in Molecular Biology. Wiley Interscience, ISBN 047 150338, 1987, whole of text;
4. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
5. Nucleic Acid Hybridization: A Practical Approach (B. D. Hames & S. J. Higgins, eds., 1985) IRL Press, Oxford, whole of text;
6. Perbal, B., A Practical Guide to Molecular Cloning (1984);
7. Animal Cell Culture: Practical Approach, Third Edition (John R. W. Masters, ed., 2000), ISBN 0199637970, whole of text;
8. J. F. Ramalho Ortigão, “The Chemistry of Peptide Synthesis” In: Knowledge database of Access to Virtual Laboratory website (Interactiva, Germany);
9. Sakakibara, D., Teichman, J., Lien, E. Land Fenichel, R. L. (1976). Biochem. Biophys. Res. Commun. 73 336-342;
10. Merrifield, R. B. (1963). J. Am. Chem. Soc. 85, 2149-2154.
11. Barany, G. and Merrifield, R. B. (1979) in The Peptides (Gross, E. and Meienhofer, J. eds.), vol. 2, pp. 1-284, Academic Press, New York.
12. Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart.
13. Bodanszky, M. (1984) Principles of Peptide Synthesis, Springer-Verlag, Heidelberg.
14. Bodanszky, M. & Bodanszky, A. (1984) The Practice of Peptide Synthesis, Springer-Verlag, Heidelberg.
15. Nagy et al eds. “Manipulating the Mouse Embryo”, Cold Spring Harbor Laboratory, 3rd Edition, 2002, ISBN 0879695749; and
16. Methods in Molecular Biology: Transgenic Mouse Methods and Protocols (Hofker and Deursen, eds., 2002), Humana Press, NJ, USA.

### SUMMARY OF INVENTION

In work leading up to the present invention, the inventors sought to identify peptide-based and/or protein-based therapeutic and prophylactic reagents for treatment of bacterial and fungal diseases in humans and animals, especially livestock animals and domestic pets. The inventors were particularly interested in developing peptide-based and protein-based therapeutics for treatment of infections by gram-negative and/or gram-positive bacteria in humans and such animals.

The inventors also sought to produce peptide-based and/or protein-based reagents for the treatment of microbial disease(s) such as one or more infections of the mammary gland (e.g., mastitis) and/or one or more infections of the respiratory system (e.g., respiratory disease in humans, and/or livestock such as pigs and/or cattle, and/or domestic animals such as dogs and/or cats) and/or one or more infections of the digestive system (e.g., clostridial intestinal disease(s) in humans, and/or livestock such as pigs and/or cattle, and/or domestic animals such as dogs and/or cats, including diarrhoea and/or hemorrhagic enteritis and/or abomasitis), and/or one or more infections of the ear(s) (e.g., otitis externa or otitis media) and/or one or more infections of the skin (e.g., dermatophytosis or Malassezia dermatitis). For applications in the dairy industry, the inventors sought to produce peptide and/or protein-based reagents having low activity against one or more gut-friendly bacteria e.g., lactobacilli.

The inventors also sought to produce peptide-based and/or protein-based reagents for the treatment of microbial disease(s) in humans such as one or more infections of the mammary gland (e.g., mastitis) and/or one or more infections of the digestive system (e.g., clostridial intestinal disease(s) such as diarrhoea and/or hemorrhagic enteritis and/or nectrotising enteritis and/or abomasitis), and/or one or more infections of the ear(s) (e.g., otitis externa or otitis media).

The inventors also sought to produce peptide-based and/or protein-based reagents for the treatment of microbial disease(s) in mammals that produce food products for human consumption such as livestock animals and/or animals that act as bioreactors e.g., pigs and/or cattle and/or poultry, such as one or more infections of the mammary gland (e.g., mastitis) and/or one or more infections of the respiratory system (e.g., bovine respiratory disease and/or swine respiratory disease) and/or one or more infections of the digestive system (e.g., clostridial intestinal disease(s) such as diarrhoea and/or hemorrhagic enteritis and/or abomasitis). In the case of applications in the dairy industry, there is also a desire for such peptide and/or protein-based reagents to have low activity against one or more gut-friendly bacteria e.g., lactobacilli.

The inventors also sought to produce peptide-based and/or protein-based reagents for the treatment of microbial disease(s) in domesticated mammals such as dogs and/or cats such as one or more infections of the respiratory system (e.g., feline respiratory disease) and/or one or more infections of the ear(s) (e.g., otitis externa or otitis media) or one or more infections of the skin (e.g., dermatophytosis or Malassezia dermatitis).

The inventors also sought to produce peptide-based and/or protein-based reagents for the treatment of microbial disease(s) mediated by Streptococcus spp. e.g., S. uberis and/or S. suis and/or S. agalactiae.

The inventors also sought to produce peptide-based and/or protein-based reagents for the treatment of microbial disease(s) mediated by enterobacteria e.g., Escherichia coli and/or Clostridium difficile and/or Clostridium perfringens.

The inventors also sought to produce peptide-based and/or protein-based reagents for the treatment of microbial disease(s) mediated by Staphylococcus spp. e.g., S. aureus and/or S. schleifen subsp. coagulans and/or S. schleiferi and/or S. intermedius and/or S. epidermis and/or S. pseudointermedin.

The inventors also sought to produce peptide-based and/or protein-based reagents for the treatment of microbial disease(s) mediated by Pasteurella spp. e.g., Mannheimia haemolytica (P. haemolytica) and/or P. multocida.

The inventors also sought to produce peptide-based and/or protein-based reagents for the treatment of microbial disease(s) mediated by Actinobacillus spp. e.g., A. pleuropneumoniae (APP) and/or A. suis

The inventors also sought to produce peptide-based and/or protein-based reagents for the treatment of microbial disease(s) mediated by Salmonella choleraesuis.

The inventors also sought to produce peptide-based and/or protein-based reagents for the treatment of microbial disease(s) mediated by Mycoplasma spp. e.g., Mycoplasma hyopneumoniae.

The inventors also sought to produce peptide-based and/or protein-based reagents for the treatment of microbial disease(s) mediated by Proteus spp. e.g., Proteus mirabili.

The inventors also sought to produce peptide-based and/or protein-based reagents for the treatment of microbial disease(s) mediated by Haemophilus spp. e.g., H. parasuis and/or or H. somnus.

The inventors also sought to produce peptide-based and/or protein-based reagents for the treatment of microbial disease(s) mediated by Pseudomonas spp.

The inventors also sought to produce peptide-based and/or protein-based reagents for the treatment of microbial disease(s) mediated by Malassezia pachydermatis.

The inventors also sought to produce peptide-based and/or protein-based reagents for the treatment of microbial disease(s) mediated by Bordetella spp. e.g., B. bronchiseptica.

More particularly, the inventors have isolated and/or produced several antimicrobial peptides having activity against at least one agent of mastitis and/or at least one agent of respiratory disease e.g., Bovine Respiratory Disease and/or Swine Respiratory and/or at least one agent of a clostridial intestinal disease and/or at least one agent of otitis externa and/or at least one agent of dermatophytosis and/or at least one agent of Malassezia dermatitis.

The peptides of the invention are useful in formulations for administration directly to humans and animals e.g., milk-producing animals and/or for ectopic expression in animals that can be used as bioreactors for their production and/or for ectopic expression in animals to protect them against infection. For example, the peptides may be expressed in one or more cells of the mammary gland (e.g., mammary cells), for example in a non-secretory or secretory form, to thereby provide prophylactic and/or therapeutic protection against the bacterium e.g., by virtue of being secreted into a mammary structure e.g., alveoli and/or lobule and/or duct and/or gland and/or teat and/or alveolar lumen and/or teat canal and/or teat cisterna and/or Furstenberg's rosette and/or by virtue of being secreted in the vicinity of a tissue such as alveolar secretory tissue (e.g., comprising cuboidal cells) and/or alveolar epithelium (e.g., basal membrane) and/or myoepithelium (e.g., comprising contractile myoepithelial cells). By way of example, the antimicrobial peptides of the invention have activity against a plurality of agents of mastitis comprising S. uberis and a further organism selected from Escherichia coli, Staphylococcus aureus, Streptococcus agalactiae, and combinations thereof, including one or two or three or all four of said organisms.

As used herein, the term “ectopic expression” shall be taken to mean expression of a peptide by means of transgenics or transient expression, the only requirement being that the peptide being expressed ectopically is not expressed in or by the cell or tissue in nature.

By “non-secretory form” is meant that an antimicrobial peptide is expressed in a cell such that it is not secreted by the cell and therefore has antimicrobial activity in the cell e.g., to provide a prophylactic or therapeutic benefit against at least one causative agent of mastitis.

By “secretory form” is meant that an antimicrobial peptide is expressed in a cell such that it is secreted by the cell in which it is expressed and therefore has antimicrobial activity outside that cell e.g., to provide a prophylactic or therapeutic benefit against at least one causative agent of mastitis. For example, a protein may be secreted to a structure of the mammary gland in the vicinity of a cell in which it is expressed, or alternatively, outside that vicinity if it is capable of being transported from a site at which it is expressed to a structure of the mammary gland e.g., by the vasculature.

The term “vicinity” shall be construed broadly to mean that a secreted peptide is found in sufficient physical proximity to a stated tissue or cell to have antimicrobial bioactivity in a therapeutic and/or prophylactic context.

Structural and functional characterization of the antimicrobial peptides of the present invention identifies sub-classes of peptides suitable for specific formulations or modes of administration to achieve a therapeutic or prophylactic benefit.

For example, the class of antimicrobial peptides described herein are active against one or more mastitis agents at salt concentrations normally found in milk products (e.g., milk, buttermilk, cream, butter and derivatives thereof) and in milk (e.g., fresh milk, pasteurised milk, homogenized milk and combinations and variants thereof such as those variants differing in milk fat composition). Such antimicrobial peptides are particularly useful for a wide range of formulations and for expression in a non-secretory or secretory form to animals at various developmental phases e.g., during prenatal development, prepubertal development, postpubertal development, pregnancy or early or late lactation.

In one example, one or more peptides of the invention has low activity against lactobacilli, especially one or more Lactis spp., in particular one or more organisms selected individually or collectively from the group consisting of: L. helveticus, L. acidophilus, L. lactis, L. bugaricus and L. citrovorum, and especially L. acidophilus. This low activity against lactobacilli suggests utility of the peptides in dairy applications e.g., dairy starter cultures, cheese production or yoghurt production, etc.

By way of example, peptides that retain their antimicrobial activity in milk are generally heat-resistant under conditions used to pasteurise milk and milk products. For example, the peptides can retain their antimicrobial activity following incubation at approximately 56° C. for 30 minutes or approximately 70° C. for 15 minutes, i.e., temperatures used for pasteurisation of milk and milk products. Such peptides are clearly suitable for being expressed ectopically in mammary glands in a secretable form for subsequent extraction from the milk e.g., prior to, during or following pasteurisation, as a bioactive agent.

As used herein, the term “milk” shall be taken to include fresh milk, pasteurised milk, homogenized milk and combinations and variants thereof such as those variants differing in milk fat composition, and milk products derived from fresh milk, pasteurised milk, homogenized milk and combinations and variants thereof such as those variants differing in milk fat composition.

The term “fresh milk” as used herein means milk that has not been pasteurised so as to kill bacteria normally present in milk.

Bioactive antimicrobial peptides extracted or purified from milk are used in pharmaceutical formulations e.g., for therapeutic and/or prophylactic treatment of infection by any one of a number of organisms against which the peptide is active i.e., not merely mastitis agents. For example, in addition to bioactivity against one or more mastitis agents, the antimicrobial peptide may have activity against any number of gram positive and/or gram negative bacteria and/or a fungus such as, for example one or more Streptococcus spp. e.g., S. uberis and/or S. suis and/or S. agalactiae and/or one or more enterobacteria e.g., Escherichia coli and/or Clostridium difficile and/or Clostridium perfringens and/or one or more Staphylococcus spp. e.g., S. aureus and/or S. schleifen subsp. coagulans and/or S. schleiferi and/or S. intermedius and/or S. epidermis and/or S. pseudointermedin and/or one or more Pasteurella spp. e.g., Mannheimia haemolytica (P. haemolytica) and/or P. multocida and/or one or more Actinobacillus spp. e.g., A. pleuropneumoniae (APP) and/or A. suis and/or one or more Mycoplasma spp. e.g., Mycoplasma hyopneumoniae and/or M. bovis and/or one or more Proteus spp. e.g., Proteus vulgaris and/or Proteus mirabili and/or one or more Haemophilus spp. e.g., H. parasuis and/or or H. somnus and/or one or more Pseudomonas spp. e.g., P. aeruginosa, and/or Malassezia pachydermatis and/or Salmonella choleraesuis and/or B. bronchiseptica. The accompanying examples also demonstrate efficacy of these peptides against one or more bacteria selected from the group consisting of S. uberis, S. suis, S. agalactiae, P. aeruginosa, E. coli, S. aureus, S. schleifen subsp. coagulans, S. schleiferi, S. epidermis, S. pseudointermedin, Mannheimia haemolytica (P. haemolytica), P. multocida, A. pleuropneumoniae (APP), H. somnus, Salmonella choleraesuis and B. bronchiseptica.

The inventors also identified a class of antimicrobial peptides having conditional bioactivity against one or more of the disease agents referred to herein, especially those effective against an agent of mastitis. By “conditional bioactivity” is meant that the antimicrobial activity of the peptide against one or more organisms is reduced or antagonized or partially or completely inhibited when contacted with milk as hereinbefore defined. Such antimicrobial peptides are particularly useful for non-milk formulations and for expression in a non-secretory or secretory form to non-lactating animals e.g., during prenatal development, prepubertal development or postpubertal development or early pregnancy. Such peptides provide the added advantage of safety in humans, because they do not require a separate inactivation process, e.g., (pasteurisation or filtration) prior to human consumption. Accordingly, milk products treated with such peptides are generally less expensive to produce.

The inventors also identified a class of antimicrobial peptides comprising an amino acid sequence selected from the group consisting of:

a) the consensus sequence TKFRNSIX₁X₂RLKNFN (SEQ ID NO: 1), wherein X₁ is a basic amino acid e.g., K or R, and wherein X₂ is N or K; and
b) a sequence having at least about 65% identity to SEQ ID NO: 7.

By way of example, peptides falling within this structural group comprise a sequence selected from the group consisting of SEQ ID NO: 10, SEQ ID NO: 14, SEQ ID NO: 15, SEQ ID NO: 16, SEQ ID NO: 17 and SEQ ID NO: 18. For the purposes of nomenclature, each of SEQ ID NO: 10, SEQ ID NO: 14, SEQ ID NO: 15, SEQ ID NO: 16, SEQ ID NO: 17 and SEQ ID NO: 18 is derived from a base peptide consisting of SEQ ID NO: 7 by mutagenesis as described herein.

The inventors also identified a class of antimicrobial peptides comprising an amino acid sequence selected from the group consisting of:

a) the consensus sequence KRGXG (SEQ ID NO: 2), wherein X is a basic amino acid e.g., R or K, or a non-polar amino acid e.g., L or F; and
b) a sequence having at least about 55% identity to SEQ ID NO: 7 or SEQ ID NO: 8.

By way of example, peptides falling within this structural group comprise a sequence selected from the group consisting of SEQ ID NO: 19, SEQ ID NO: 20, SEQ ID NO: 22 and SEQ ID NO: 31. For the purposes of nomenclature, each of SEQ ID NO: 19, SEQ ID NO: 20 and SEQ ID NO: 22 is derived from a base peptide consisting of SEQ ID NO: 8 by mutagenesis, and SEQ ID NO: 31 is derived from a consensus sequence for antimicrobial C-terminal fragments of cathelicidin proteins as described herein.

The inventors also identified a class of antimicrobial peptides comprising an amino acid sequence selected from the group consisting of:

a) the consensus sequence MVKRGXGE (SEQ ID NO: 3), wherein X is a non-polar amino acid e.g., L or F; and
b) a sequence having at least about 55% identity to SEQ ID NO: 7 or SEQ ID NO: 8.

By way of example, peptides falling within this structural group comprise a sequence selected from the group consisting of SEQ ID NO: 19, SEQ ID NO: 20 and SEQ ID NO: 22.

The inventors also identified a class of antimicrobial peptides comprising an amino acid sequence selected from the group consisting of:

a) the consensus sequence IX₁X₂TLX₃NFX₄X₅, (SEQ ID NO: 4) wherein X₁ and X₃ are each a basic amino acid, e.g., K or R, X₂ and X₄ are each K or N, and X₅ is a non-polar amino acid e.g., F or L; and
b) a sequence having at least about 54% identity to SEQ ID NO: 8.

By way of example, peptides falling within this structural group comprise a sequence selected from the group consisting of SEQ ID NO: 11, SEQ ID NO: 12, SEQ ID NO: 15, SEQ ID NO: 16, SEQ ID NO: 19, SEQ ID NO: 20 and SEQ ID NO: 21. For the purposes of nomenclature, each of SEQ ID NO: 11, SEQ ID NO: 12, SEQ ID NO: 15, SEQ ID NO: 16, SEQ ID NO: 19, SEQ ID NO: 20 is derived from a base peptide consisting of SEQ ID NO: 8 by mutagenesis as described herein.

The inventors also identified a class of antimicrobial peptides comprising an amino acid consensus sequence set forth in SEQ ID NO: 88. For the purposes of nomenclature, the sequence set forth in SEQ ID NO: 88 is derived by alignment of the sequences of antimicrobial peptides set forth in SEQ ID NO: 7 and 8 and derivatives thereof having enhanced antimicrobial activity.

Peptides falling within the parameters of each of these structural classes are produced without undue experimentation, e.g., using standard synthetic techniques for producing peptides, and determining antimicrobial activity e.g., by growth inhibition assay.

The inventors also produced the antimicrobial exemplified by amino acid sequences set forth in SEQ ID Nos: 23-32 by mutagenesis based on a consensus sequence for antimicrobial C-terminal fragments of cathelicidin proteins as described herein.

Of the sequences set forth in SEQ ID Nos: 10-32, sequences set forth in SEQ ID Nos: 10-13 and 23-32 were shown to have enhanced growth inhibition relative to their parent base peptide(s) i.e., SEQ ID NO: 7 and/or SEQ ID NO: 8.

The inventors have also derived retro-analogs and retro-inverso analogs of the sequences set forth in each of SEQ ID Nos: 7-22.

### Specific Embodiments

In one example, the present invention provides an antimicrobial peptide or an analog or derivative thereof, said peptide, analog or derivative having antimicrobial activity against one or more Streptococcus spp. e.g., S. uberis and/or S. suis and/or S. agalactiae and/or one or more enterobacteria e.g., Escherichia coli and/or Clostridium difficile and/or Clostridium perfringens and/or one or more Staphylococcus spp. e.g., S. aureus and/or S. schleifen subsp. coagulans and/or S. schleiferi and/or S. intermedius and/or S. epidermis and/or S. pseudointermedin and/or one or more Pasteurella spp. e.g., Mannheimia haemolytica (P. haemolytica) and/or P. multocida and/or one or more Actinobacillus spp. e.g., A. pleuropneumoniae (APP) and/or A. suis and/or one or more Mycoplasma spp. e.g., Mycoplasma hyopneumoniae and/or M. bovis and/or one or more Proteus spp. e.g., Proteus mirabili and/or one or more Haemophilus spp. e.g., H. parasuis and/or or H. somnus and/or one or more Pseudomonas spp. e.g., P. aeruginosa, and/or Malassezia pachydermatis and/or Salmonella choleraesuis and/or B. bronchiseptica.

In another example, the present invention provides an antimicrobial peptide or an analog or derivative thereof, said peptide, analog or derivative having antimicrobial activity against one or more bacteria selected from the group consisting of S. uberis, S. suis, S. agalactiae, P. aeruginosa, E. coli, S. aureus, S. schleifen subsp. coagulans, S. schleiferi, S. epidermis, S. pseudointermedin, Mannheimia haemolytica (P. haemolytica), P. multocida, A. pleuropneumoniae (APP), H. somnus, Salmonella choleraesuis and B. bronchiseptica. Peptides having activity against combinations of said bacteria, including peptides having activity against one or two or three or four or five or six or seven or eight or nine or ten or eleven or twelve or thirteen or fourteen or fifteen or sixteen of said organisms in any combination are clearly encompassed by the present invention.

In another example, the present invention provides an antimicrobial peptide or an analog or derivative thereof, said peptide, analog or derivative having antimicrobial activity against Streptococcus uberis, and preferably a plurality of agents of mastitis comprising S. uberis and a further organism selected from S. suis, S. agalactiae, P. aeruginosa, E. coli, S. aureus, S. schleifen subsp. coagulans, S. schleiferi, S. epidermis, S. pseudointermedin, Mannheimia haemolytica (P. haemolytica), P. multocida, A. pleuropneumoniae (APP), H. somnus, Salmonella choleraesuis and B. bronchiseptica and any combinations thereof, including one or two or three or four or five or six or seven or eight or nine or ten or eleven or twelve or thirteen or fourteen or fifteen or all sixteen of said organisms

In another example, one or more antimicrobial peptides of the invention has low activity against lactobacilli, especially one or more Lactis spp., in particular one or more organisms selected individually or collectively from the group consisting of L. helveticus, L. acidophilus, L. lactis, L. bugaricus and L. citrovorum, and especially L. acidophilus.

As used herein, the term “antimicrobial” shall be taken to mean that the peptide or analog or derivative thereof is capable of killing a microorganism and/or preventing growth of a microorganism, i.e., the peptide has microbicidal activity and/or microbiostatic activity. Methods for determining the antimicrobial activity of a peptide or analog or derivative thereof will be apparent to the skilled artisan and/or described herein. For example, the peptide or analog or derivative is applied to a substrate upon which a microorganism has been previously grown and, after a suitable period of time, the level of growth inhibition and/or cell death of the microorganism is determined. The term “microorganism” encompasses any microscopic organism, preferably S. uberis and, optionally includes any other microscopic organism, preferably a microorganism that causes mastitis and/or respiratory disease e.g., Bovine Respiratory Disease and/or Swine Respiratory Disease, and/or clostridial intestinal disease and/or otitis externa and/or dermatophytosis and/or Malassezia dermatitis, and preferably a bacterium that causes mastitis and/or respiratory disease e.g., Bovine Respiratory Disease and/or Swine Respiratory Disease, and/or clostridial intestinal disease and/or otitis externa. Exemplary microorganisms in this context are bacteria e.g., gram-positive bacteria or gram-negative bacteria e.g., S. uberis and/or S. aureus and/or S. agalactiae and/or E. coli and/or S. suis and/or P. aeruginosa and/or S. schleifen subsp. coagulans and/or S. schleiferi and/or S. epidermis and/or S. pseudointermedin and/or Mannheimia haemolytica (P. haemolytica) and/or P. multocida and/or A. pleuropneumoniae (APP) and/or H. somnus and/or Salmonella choleraesuis and/or B. bronchiseptica and any combinations thereof, including one or two or three or four or five or six or seven or eight or nine or ten or eleven or twelve or thirteen or fourteen or fifteen or all sixteen of said organisms.

As used herein, the term “analog” includes a peptide modified by varying the amino acid sequence, e.g., by substituting an amino acid with a naturally-occurring amino acid and/or by substituting an amino acid with a non-naturally occurring amino acid and/or by addition of a naturally-occurring amino acid and/or by addition of a non-naturally occurring amino acid and/or by deleting one or more amino acids. Analogs also include peptidomimetics, e.g., in which one or more peptide bonds have been modified. Preferred analogs include an analog of a peptide as described according to any embodiment hereof comprising one or more non-naturally-occurring amino acid analogs; an analog of a peptide described according to any embodiment hereof comprising one or more D-amino acids; an analog of a peptide described according to any embodiment hereof in which at least one peptide bond is replaced by a non-peptide bond (i.e., a peptidomimetic); an isostere of a peptide as described according to any embodiment hereof; a retro-peptide analog of a peptide as described according to any embodiment hereof; and a retro-inverted peptide analog of a peptide as described according to any embodiment hereof.

As used herein the term “derivative” with reference to a stated peptide shall be taken to include e.g., a fragment or processed form of the stated peptide, a variant or mutant comprising one or more amino acid substitutions, deletions of additions relative to the stated peptide, a fusion protein comprising the stated peptide or a peptide comprising one or more additional non-peptide components relative to the stated peptide e.g., a chemical component, e.g., polyethylene glycol (PEG).

Preferred fusion proteins encompassed by the present invention include a fusion protein comprising a peptide, analog or derivative as described according to any embodiment hereof fused or linked to another peptide, polypeptide or protein, e.g., a fusion protein comprising a plurality of peptides, polypeptides or analogs of the present invention, optionally separated by a protease cleavage site. A further example of a fusion protein comprises one or more peptide, analogs or derivatives fused to a heterologous protein to thereby display the peptide(s) analog(s) and/or derivative(s) within said heterologous protein. Another example of a fusion protein comprises a peptide, analog or derivative as described according to any embodiment hereof fused or linked to an epitope to facilitate detection or isolation of the peptide. An exemplary epitope is a FLAG epitope or a V5 epitope or an HA epitope. In another example, a fusion protein comprises a plurality of peptides and/or analogs and/or derivatives as described according to any embodiment hereof.

In one example, the antimicrobial peptide or analog or derivative thereof retains its antimicrobial activity at salt concentrations normally found in a milk product (e.g., milk, buttermilk, cream, butter and derivatives thereof) and/or in milk (e.g., fresh milk, pasteurised milk, homogenized milk and combinations and variants thereof such as those variants differing in milk fat composition). For example, the antimicrobial peptide or analog or derivative thereof has antimicrobial activity at a salt concentration between about 20 mM NaCl and about 100 mM NaCl. Preferably the antimicrobial peptide or analog or derivative thereof has antimicrobial activity at a salt concentration between about 25 mM NaCl and about 75 mM NaCl. More preferably the antimicrobial peptide or analog or derivative thereof has antimicrobial activity at a salt concentration between about 25 mM NaCl and about 50 mM NaCl. Even more preferably the antimicrobial peptide or analog or derivative thereof has antimicrobial activity at a salt concentration of about 30 mM NaCl.

Alternatively, or in addition, the antimicrobial peptide or analog or derivative thereof has antimicrobial activity following heating to a temperature at which milk or a milk product is generally pasteurized. For example, the antimicrobial peptide or analog or derivative thereof has antimicrobial activity following incubation at approximately 56° C. for 30 minutes or approximately 70° C. for 15 minutes. In this respect, the level of antimicrobial activity of the peptide or analog or derivative thereof following heating need not be the same as the level of activity before treatment. For example, the level of activity can be enhanced, reduced or approximately the same following heating.

Alternatively, or in addition, the antimicrobial activity of said peptide or analog or derivative thereof is completely or partially reduced or inhibited following heating to a temperature at which milk or a milk product is generally pasteurized. For example, the antimicrobial activity of said peptide or analog or derivative thereof is completely or partially reduced or inhibited following incubation at approximately 56° C. for 30 minutes or approximately 70° C. for 15 minutes.

Alternatively, or in addition, the antimicrobial activity of the antimicrobial peptide and/or analog and/or derivative is reduced or antagonized or partially or completely inhibited when contacted with milk.

Alternatively or in addition, the antimicrobial peptide or analog or derivative is resistant to a protease expressed and/or active in milk or a mammary gland or cell or tissue thereof.

Alternatively or in addition, the antimicrobial peptide or analog or derivative thereof comprises an amino acid sequence selected individually or collectively from the group consisting of:

a) the consensus sequence TKFRNSIX₁X₂RLKNFN (SEQ ID NO: 1), wherein X₁ is a basic amino acid e.g., K or R, and wherein X₂ is N or K; and/or
b) the consensus sequence KRGXG (SEQ ID NO: 2), wherein X is a basic amino acid e.g., R or K, or a non-polar amino acid e.g., L or F; and/or
c) the consensus sequence MVKRGXGE (SEQ ID NO: 3), wherein X is a non-polar amino acid e.g., L or F; and/or
d) the consensus sequence IX₁X₂TLX₃NFX₄X₅, (SEQ ID NO: 4) wherein X₁ and X₃ are each a basic amino acid, e.g., K or R, X₂ and X₄ are each K or N, and X₅ is a non-polar amino acid e.g., F or L; and/or
e) the consensus sequence set forth in SEQ ID NO: 88,
wherein the amino acid sequence of said antimicrobial peptide is at least about 50% identical to SEQ ID NO: 7 and/or SEQ ID NO: 8.

Alternatively or in addition, the antimicrobial peptide, derivative or analog possesses enhanced antimicrobial activity against one or more bacterial agents, e.g., one or more bacterial agents described herein, compared to an antimicrobial peptide comprising the sequence of SEQ ID NO: 7 or SEQ ID NO: 8 under the same conditions.

Alternatively or in addition, the antimicrobial peptide, derivative or analog comprises an amino acid sequence other than SEQ ID NO: 7 or SEQ ID NO: 8 or SEQ ID NO: 9 hereof.

Alternatively or in addition, the antimicrobial peptide comprises a sequence set forth in any one of SEQ ID NOs: 10-32 or an analog or derivative thereof having antimicrobial activity.

Preferred peptides, and analogs and derivatives comprise a sequence selected from SEQ ID Nos: 10-13 and 23-32, more preferably SEQ ID Nos: 10-13, 24, 25, 28 and 30 and analogs and derivatives thereof.

In one example, the analog is a retro-peptide analog. The skilled artisan will be aware that a retro-peptide analog is a peptide analog in which the sequence of at least two amino acids is reversed. Preferably a retro-peptide analog as described according to any embodiment hereof is a peptide analog in which the sequence of all amino acids in the analog is reversed. In one example, a retro-peptide analog as described according to any embodiment hereof comprises a sequence set forth in any one of SEQ ID NOs: 33-58, preferably 36-58, more preferably 36-39 and 49-58, and still more preferably SEQ ID Nos: 36-39, 50, 51, 54 and 56.

In another example, the analog is a retro-inverted peptide analog. The skilled artisan will also be aware that a retro-inverted peptide analog is a peptide analog in which the sequence of at least two amino acids is reversed and those amino acids are D-amino acids. Preferably a retro-inverted peptide analog as described according to any embodiment hereof is a peptide analog in which the sequence of all amino acids in the analog is reversed and all amino acids in the analog other than glycine are D-amino acids. In one example, a retroinverted-peptide analog as described according to any embodiment hereof comprises a sequence set forth in any one of SEQ ID NOs: 59-83, preferably 61-83, more preferably 61-64 and 74-83, and still more preferably SEQ ID Nos: 61-64, 75, 76, 79 and 81.

The present invention also encompasses a complex of peptides and/or analogs and/or derivatives of the present invention. Without being bound by theory or mode of action or suggesting that a complex is necessary for performance of the present invention, such a complex is useful for enhancing the antimicrobial activity of an antimicrobial peptide or analog or derivative of the invention. For example, the present invention provides a complex of the same peptide, analog or derivative. Alternatively, the present invention provides a complex or aggregate comprising a plurality of different peptides and/or analogs and/or derivatives of the invention. Such complex or aggregate may comprise additional antimicrobial peptides known in the art. Synergistic combinations of two or more peptides, analogs or derivatives of the present invention are also encompassed.

In another example, the present invention also provides a composition comprising an amount of a peptide, analog derivative, fusion protein or complex as described herein in any embodiment and a suitable carrier or excipient. Such compositions may also comprise a plurality of peptides, analogs or derivatives described according to any example hereof, including combinations of peptides, analogs or derivatives wherein the combination provides an additive or non-additive i.e., synergistic, effect against one or more microbial agents compared to the individual peptide, analog or derivate components of the combination. For example, the present invention provides a pharmaceutical composition. Such a composition may take any of a number of forms, such as, for example, a solution (e.g., a spray solution or a pharmaceutical solution, e.g., a nasal spray solution or syrup), an aerosol, a cream, a lotion, a gel or a powder. Suitable compositions will be apparent to the skilled artisan based on the description herein.

Preferably the composition comprises an amount of a peptide and/or analog and/or derivative and/or complex sufficient to kill and/or prevent growth of S. uberis and, optionally, further organism selected from S. aureus and/or S. agalactiae and/or E. coli and/or S. suis and/or P. aeruginosa and/or S. schleifen subsp. coagulans and/or S. schleiferi and/or S. epidermis and/or S. pseudointermedin and/or Mannheimia haemolytica (P. haemolytica) and/or P. multocida and/or A. pleuropneumoniae (APP) and/or H. somnus and/or Salmonella choleraesuis and/or B. bronchiseptica and any combinations thereof, including one or two or three or four or five or six or seven or eight or nine or ten or eleven or twelve or thirteen or fourteen or fifteen or all sixteen of said organisms. Preferably the composition comprises an amount of a peptide and/or analog and/or derivative and/or complex to treat or prevent mastitis and/or respiratory disease e.g., Bovine Respiratory Disease and/or Swine Respiratory Disease, and/or clostridial intestinal disease and/or otitis externa and/or dermatophytosis and/or Malassezia dermatitis, and preferably to treat mastitis and/or respiratory disease e.g., Bovine Respiratory Disease and/or Swine Respiratory Disease, and/or clostridial intestinal disease and/or otitis externa. Again, pluralities of peptides, analogs or derivative are encompassed, including synergistic combinations.

As used herein, the term “suitable carrier or excipient” shall be taken to mean a compound or mixture thereof that is suitable for use in a formulation for administration to a cell, tissue, organ or subject. In one example, the carrier or excipient is suitable for administration to a mammary gland of cell or tissue thereof albeit not necessarily limited in use to that context.

A suitable carrier or excipient may be an “intra-mammary carrier or excipient”. In this respect, an “intra-mammary carrier or excipient” is compound or mixture thereof that is described in the art only with reference to a use in a composition for administration to a mammary gland or tissue or cell thereof. Such a carrier or excipient for production of a composition for treatment or prophylaxis of mastitis and/or respiratory disease e.g., Bovine Respiratory Disease and/or Swine Respiratory Disease, and/or clostridial intestinal disease and/or otitis externa and/or dermatophytosis and/or Malassezia dermatitis, and preferably mastitis and/or respiratory disease e.g., Bovine Respiratory Disease and/or Swine Respiratory Disease, and/or clostridial intestinal disease and/or otitis externa.

Alternatively, the carrier or excipient is a carrier or excipient for intramammary application”. The term “carrier or excipient for intramammary application” shall be taken to mean a compound or mixture thereof that is suitable for application to a mammary gland or cell or tissue thereof, and which may be suitable for use in other contexts.

In another example, the present invention also provides a solid surface coated with or having adsorbed thereto the peptide and/or analog and/or derivative, complex or composition as described herein in any embodiment. For example, the present invention provides a bead or implant coated with the peptide and/or analog and/or derivative and/or complex of the invention, e.g., an automatic milking device and/or an intramammary device.

As discussed herein above an antimicrobial peptide and/or analog and/or derivative of the present invention is suitable for ectopic expression in one or more cells of a mammary gland, e.g., to provide prophylactic and/or therapeutic protection against S. uberis and/or to treat or prevent mastitis and/or to produce the peptide and/or analog and/or derivative, e.g., for therapeutic or prophylactic purposes. Accordingly, the another example of the present invention provides means for expressing a peptide and/or analog and/or derivative as described according to any embodiment hereof in a mammary gland or cell or tissue thereof. An exemplary expression construct comprises nucleic acid encoding a peptide and/or analog and/or derivative as described according to any embodiment hereof operably linked to a promoter that confers expression on said nucleic acid in a mammary gland or cell or tissue thereof. In one example, the expression construct of the present invention comprises a nucleic acid comprising a sequence set forth in any one of SEQ ID NOs: 84-88, 90 or 91, or alternatively, a sequence capable of encoding any one or more peptides or retro-peptides comprising an amino acid sequence set forth in SEQ ID Nos: 10-58.

As used herein, the term “promoter” is to be taken in its broadest context and includes transcriptional regulatory sequences of a classical genomic gene, including the TATA box which is required for transcription initiation, with or without a CCAAT box sequence and additional regulatory elements (e.g., upstream activating sequences, enhancers and silencers) which confer gene expression in a mammary gland or cell or tissue thereof. A promoter is usually, but not necessarily, positioned upstream, or 5′, of a structural gene, upon which it confers expression. Furthermore, the regulatory elements comprising a promoter are usually positioned within 2 kb of the start site of transcription of a gene. Preferred promoters can contain additional copies of one or more specific regulatory elements to further enhance expression and/or alter the spatial expression and/or temporal expression of said nucleic acid. Preferably the promoter preferentially or specifically confers expression on the nucleic acid in a mammary gland or cell or tissue thereof.

As used herein, the term “operably linked to” means positioning a promoter relative to a nucleic acid, e.g., a transgene such that expression of the nucleic acid is controlled by the promoter. For example, a promoter is generally positioned 5′ (upstream) to the nucleic acid, the expression of which it controls. To construct heterologous promoter/nucleic acid combinations (i.e., an expression construct of the present invention), it is generally preferred to position the promoter at a distance from the gene transcription start site that is approximately the same as the distance between that promoter and the nucleic acid it controls in its natural setting, i.e., the gene from which the promoter is derived. As is known in the art, some variation in this distance can be accommodated without loss of promoter function.

Suitable methods for linking nucleic acids will be apparent to the skilled artisan and/or described herein and include enzymatic ligation, e.g., T4 DNA ligase, topoisomerase-mediated ligation e.g., using Vaccinia DNA topoisomerase I, recombination in cis or trans, e.g., using a recombinase or by random integration, amplification from one or more primer sequences including primer extension means, amplification from a vector, or chemical ligation, e.g., cyanogen bromide-mediated condensation of nucleic acids.

As used herein, and unless the context requires otherwise, the word “confer” and variations thereof such as “conferring” shall be taken to mean the ability of a promoter, for example in the context of other factors such as DNA conformation and/or cis-acting DNA sequence(s) and/or trans-acting factor(s) and/or signalling pathway(s) and/or transcript structure and/or transcript processing, to produce expression or a pattern of expression of nucleic acid to which the promoter is operably-linked in a mammary gland or cell or tissue thereof, e.g., in response to one or more developmental and/or environmental and/or hormonal and/or other stimuli that would normally elicit the expression or pattern of expression for nucleic acid to which the promoter is operably-connected in its native context.

Preferably the promoter confers expression on the nucleic acid operably-linked thereto at a time at which a mammal is at risk of being infected by a microorganism that causes mastitis and/or is at risk of developing mastitis, e.g., during pregnancy and before lactation commences and/or at the time of or following giving birth, and/or during involution and/or during lactation.

Suitable promoters will be apparent to the skilled artisan and include, for example, a β-casein gene promoter (e.g., comprising a sequence set forth in SEQ ID NO: 92) or a prolactin-inducible mammary specific promoter (e.g., comprising a sequence set forth in SEQ ID NO: 93) or a α-lactalbumin gene promoter (e.g., comprising a sequence set forth in SEQ ID NO: 94) or a whey acidic protein (WAP) gene promoter (e.g., comprising a sequence set forth in SEQ ID NO: 95 or 96 or 97) or a β-lactoglobulin gene promoter (e.g., comprising a sequence set forth in SEQ ID NO: 98 or 99 or 100). Each of these promoters confers expression on a nucleic acid operably linked thereto at least in a mammary epithelial cell at least during lactation. A lactalbumin promoter also confers expression on a nucleic acid linked thereto in at least mammary epithelial cells during pregnancy. In another example, the promoter is a nuclear factor-κB (NF-κB) responsive promoter. In this respect, NF-κB is expressed at increased levels in mammary epithelial cells during mastitis and/or during involution. For example, the promoter is derived from a lactoferrin gene, e.g., a bovine lactoferrin gene. Such a promoter confers expression on a nucleic acid operably-linked thereto in a mammary cell, and this expression is increased during infection, e.g., by a microorganism that causes mastitis.

The expression construct may also comprise one or more intron sequences positioned downstream of the promoter and transcription start site and optionally downstream of a translation start site for the encoded protein to be expressed. The intron sequence may enhance transcript stability. Preferred introns must be capable of being processed form a primary transcript in the target cell or tissue in which the fusion protein is to be expressed and will generally be employed for expressing an antimicrobial peptide, analog or derivative of the invention or a fusion protein comprising same in a eukaryotic cell or tissue. Preferred introns are intron 1 sequences derived from native genomic genes. For example, preferred intron 1 sequences for expression in bovine cells and tissues are described by Mossallam et al., J. App. Sci. 3(11), 1400-1406 (2007) incorporated herein by reference.

In one example, an expression construct of the present invention further comprises a sequence encoding a signal peptide that provides for expression of a fusion protein comprising an N-terminal signal peptide and C-terminal antimicrobial peptide, analog or derivative. Preferably, the signal peptide directs secretion of the peptide and/or analog and/or derivative of the invention into milk. Accordingly, the encoded antimicrobial peptide or analog or derivative is in secretable form. Suitable signal peptides will be apparent to the skilled artisan and/or described herein and include an α-1 lactalbumin signal peptide (e.g., comprising a sequence set forth in SEQ ID NO: 101) or a αS-1 casein signal peptide (e.g., comprising a sequence set forth in SEQ ID NO: 102) or a β-lactoglobulin signal peptide (e.g., comprising a sequence set forth in SEQ ID NO: 103). Generally, a sequence encoding a signal peptide is positioned between the promoter sequence and sequence encoding the antimicrobial peptide, analog or derivative.

Alternatively, or in addition, an expression construct of the present invention comprises a sequence encoding a prepro sequence of a cathelicidin protein that when linked in the same reading frame to a sequence encoding an antimicrobial peptide, analog or derivative described according to example hereof provides for expression of a fusion protein comprising an N-terminal prepro sequence and C-terminal antimicrobial peptide, analog or derivative. Preferably, the fusion protein is subsequently processed by cellular protease(s) to release the mature and bioactive antimicrobial peptide, analog or derivative. By expressing the antimicrobial peptide, analog or derivative as a prepro protein, the stability of the encoded peptide, analog or derivative and/or resistance of the encoded peptide, analog or derivative to proteolysis may be enhanced. Suitable prepro sequences of cathelicidin proteins are readily available to the skilled artisan and include milk-expressed or mammary gland expressed proteins from any one of a number of mammals, including the Macropus eugenii sequences set forth in SEQ ID Nos: 104 and 105. Any one of the bovine sequences set forth in SEQ ID Nos: 106-111 may also be employed. Generally, a sequence encoding a cathelicidin prepro sequence is positioned between the promoter sequence and sequence encoding the antimicrobial peptide, analog or derivative. When a signal sequence is also present in such a construct, it is preferred to position the protein-encoding elements downstream of a promoter sequence and in a configuration that provides for expression of a fusion protein comprising N-terminal signal peptide and C-terminal antimicrobial peptide, analog or derivative with an intervening cathelicidin prepro sequence. Alternatively, the protein-encoding elements are positioned downstream of a promoter sequence and in a configuration that provides for expression of a fusion protein comprising N-terminal cathelicidin prepro sequence and C-terminal antimicrobial peptide, analog or derivative with an intervening signal peptide. Alternatively, the protein-encoding elements are positioned downstream of a promoter sequence and in a configuration that provides for expression of a fusion protein comprising N-terminal cathelicidin prepro sequence and C-terminal signal peptide with an intervening antimicrobial peptide, analog or derivative.

In another example, the expression construct of the present invention comprises a sequence encoding a fusion protein comprising a plurality of antimicrobial peptides and/or a plurality of analogs thereof and/or a plurality of derivatives thereof. For example, the fusion protein comprises at least about 2 or 3 or 4 or 5 or 6 or 7 or 8 or 9 or 10 antimicrobial peptides and/or analogs and/or derivatives thereof. As with constructs comprising a single copy of an antimicrobial peptide, analog or derivative of the invention, constructs comprising a plurality of peptides, analogs or derivatives may also include one or more sequences encoding signal sequence(s) and/or one or more sequences encoding prepro sequence(s) of cathelicidin protein(s).

It is also within the scope of the present invention for an expression construct to include one or more sequences encoding recognition sites for protease(s) e.g., a serine protease(s) or cysteine protease(s), such as positioned between a sequence encoding a prepro sequence and a sequence encoding the antimicrobial peptide, analog or derivative, or alternatively, positioned between a sequence encoding a signal sequence and a sequence encoding the antimicrobial peptide, analog or derivative, or alternatively, positioned between each sequence encoding an antimicrobial peptide, analog or derivative in a construct comprising a plurality of peptides, analogs or derivatives. Preferred recognition sequences for proteases will not be present in the antimicrobial peptide, analog or derivative moiety of the fusion protein, or if present in that moiety, will not be high affinity site(s) for proteases present in a cell, tissue or other protease-containing environment in which the fusion protein is expressed. For example, a sequence encoding the enterokinase recognition sequence (D-D-D-D-K; SEQ ID NO: 112) and/or a sequence encoding a recognition sequence for a protease selected from furin (R-V-R-R), filaggrin (R-K-R-R), HGF-SF (K-Q-L-R), MT-SP1/matriptase (R-Q-A-R), PAR2 (S-K-G-R) or uPA/urokinase (P-R-F-K) may be employed. For fusion proteins expressed as prepro proteins in milk or with signal sequences targeting the fusion protein or its processed form to milk, it is preferred to employ a sequence encoding a recognition sequence for a protease present in milk e.g., plasmin, plasminogen, plasminogen activator, thrombin, cathepsin D, acid milk protease or aminopeptidase. High affinity recognition sequences for the human serine proteases plasmin, thrombin, factor Xa, plasmin and urokinase plasminogen activator are known in the art e.g., Gosalia et al., Proteomics 5, 1292-1298 (2005) incorporated herein by reference. High affinity recognition sequences for the human MT-SP1/matriptases proteases plasmin, thrombin, factor Xa, plasmin and urokinase plasminogen are also known in the art e.g., Proc. Natl. Acad. Sci (USA) 104(14), 5771-5776 (2007) incorporated herein by reference. In this manner, following expression of the fusion protein, individual peptides and/or analogs and/or derivatives are released by protease cleavage to thereby exert an antimicrobial activity, e.g., to reduce or prevent or treat mastitis.

Alternatively, each peptide or analog or derivative is separated from another peptide or analog or derivative by a cleavage site of a protease that is not expressed or active in a target cell, tissue or other protease-containing environment in which the fusion protein is expressed e.g., mammary gland or cell or tissue thereof or secretion thereof. In this example, a fusion protein comprising the antimicrobial peptide, analog or derivative and other protein elements e.g., signal sequence and/or prepro sequence separated by protease recognition and cleavage site(s) is isolated from the cell or tissue in which it is expressed milk, and cleaved with the relevant protease recognizing the protease recognition and cleavage site to thereby release bioactive antimicrobial peptides and/or analogs and/or derivatives, e.g., for therapeutic or prophylactic use. For example, TEV protease may be employed in such applications.

In another example, the present invention provides an expression vector comprising an expression construct as described according to any embodiment hereof. The skilled artisan will be aware that in addition to the expression construct of the present invention, an expression vector generally comprises one or more sequences to permit it to be maintained in a cell e.g., one or more selectable marker genes e.g., to confer antibiotic resistance on a cell comprising the expression vector, and one or more origins of replication e.g., for replication in bacterial cells and/or yeast cells. An expression vector may also include one or more recombinase site sequences to permit excision of a portion of its DNA in a cell and/or to facilitate integration into host cell DNA. Expression vectors encompassed by the present invention include a plasmid, bacteriophage, phagemid, cosmid, virus, sub-genomic or genomic fragment, bacterial artificial chromosome, yeast artificial chromosome or other nucleic acid capable of maintaining and or replicating heterologous DNA in an expressible format. Selection of appropriate vectors is within the knowledge of those having skill in the art.

Expression constructs and/or expression vectors as described according to any embodiment hereof are also useful for producing a recombinant cell comprising said expression vector or expression construct. Accordingly, the present invention provides a cell comprising an expression vector or expression vector as described according to any embodiment hereof. In one example, the cell is a zygote or an oocyte or an embryonic cell or a stem cell, e.g., a pluripotent cell.

In another example, the present invention also provides a genetically-modified non-human mammal comprising an expression construct or an expression vector as described according to any embodiment hereof. Preferably said genetically-modified non-human mammal expresses a peptide and/or analog and/or derivative as described according to any embodiment hereof in a mammary gland or a cell or tissue thereof and/or a secretion of a mammary gland or cell or tissue thereof, e.g., milk.

As used herein, the term “genetically-modified” shall be taken to mean a non-human mammal that comprises genetic material additional to the naturally-occurring nucleic acid within said non-human mammal, i.e., nucleic acid encoding an antimicrobial peptide and/or analog and/or derivative as described according to any embodiment hereof.

In this respect, the present invention is not to be limited to a non-human mammal comprising the expression construct or expression vector within their genome. Rather, the present invention also encompasses a genetically-modified non-human mammal comprising an episomal expression construct, e.g., within an artificial chromosome or expression vector or viral nucleic acid. The present invention also encompasses a genetically-modified non-human mammal in which a transgene is only within one or more cells within a mammary gland, i.e., either integrated into the genome of the cell within the mammary gland or episomal within the cell within the mammary gland. A genetically-modified non-human mammal comprising an episomal expression construct within a cell of the mammary gland is produced, for example, using a virus and/or by administration of naked DNA to a mammary gland cell. Suitable methods for such administration are described herein.

Preferably the genetically-modified non-human mammal comprises a genetic construct as described according to any embodiment hereof within its genome. However, the present invention is not to be limited by the means of integration of the nucleic acid into the genome of the non-human mammal. For example, the nucleic acid can be randomly integrated into the animal's genome or integrated at a pre-determined site by homologous recombination (e.g., “knocked-in”).

As used herein, the term “non-human mammal” shall be taken to refer to any endothermic animal comprising at least one mammary gland, other than a human. Preferably the non-human mammal produces milk that is consumed by humans, e.g., that is collected and distributed for human consumption. For example, the non-human animal is a ruminant mammal. By “ruminant mammal” is meant any artiodactyl mammal (i.e., of the order Artiodactyla) that digests its food in two steps, first by eating raw material and regurgitating a semi-digested form known as cud from within their first stomach (known as the rumen) and again chewing the cud to break down plant matter therein and stimulate digestion. Ruminants include cattle, goats, sheep, camels, alpacas, llamas, giraffes, American Bison, European bison, yaks, water buffalo, deer, wildebeest and antelope. Preferably the non-human mammal is of the suborder Ruminantia.

Preferably a non-human mammal is a bovine mammal, preferably Bos taurus or a cattle cross, an ovine mammal, preferably Ovis aries, a caprine mammal, preferably Capra hircus, a camel, preferably Camelus dromedarius, a Bubalis mammal, preferably Bubalus bubalis or a Rangifer mammal, preferably Rangifer tarandus. Preferably the non-human mammal is a bovine mammal, preferably Bos taurus, an ovine mammal, preferably Ovis aries, a caprine mammal, preferably Capra hircus. More preferably the non-human mammal is a bovine mammal, preferably Bos taurus.

Preferably the genetically-modified non-human mammal expresses a sufficient level of an antimicrobial peptide or an analog or derivative thereof encoded by the expression construct or expression vector in a mammary gland or cell or tissue thereof to kill or prevent growth of S. uberis and, optionally one or more additional microorganisms selected from S. aureus, S. agalactiae and E. coli and combinations thereof in the mammary gland or cell or tissue thereof, and/or to treat or prevent mastitis.

In another example, the present invention additionally provides a zygote or an embryo or an offspring of a genetically-modified non-human mammal as described according to any embodiment hereof, wherein said zygote or embryo or offspring comprises an expression construct or expression vector as described according to any embodiment hereof. In this respect, the present invention contemplates a zygote or embryo or offspring that is homozygous or heterozygous for an expression construct as described according to any embodiment hereof. Furthermore, the present invention contemplates zygotes, embryos or offspring that are inbred (and, as a consequence, substantially isogenic compared to their parents) or outbred. Methods for producing zygotes, embryos or offspring of a genetically-modified non-human mammal, e.g., by breeding or in vitro fertilization or intracellular sperm injection will be apparent to the skilled artisan.

In another example, the present invention provides reproductive material from a genetically-modified non-human mammal as described according to any embodiment hereof, wherein said reproductive material comprises an expression construct or expression vector as described according to any embodiment hereof. For example, the reproductive material is an ovary or a part thereof comprising an oocyte or precursor thereof, or an oocyte, or a precursor of an oocyte, or a testis or a part thereof comprising a sperm cell or precursor thereof, or an epididymis comprising a sperm cell, or a sperm cell, or a precursor of a sperm cell.

In another example, the present invention also provides a cell or a tissue derived or isolated from a transgenic animal as described according to any embodiment hereof wherein said cell comprises an expression construct or expression vector of the present invention. For example, the cell is a pluripotent cell or a totipotent cell or a mammary gland cell or a precursor cell of a mammary gland cell.

In another example, the present invention also provides a method for producing a genetically-modified non-human mammal expressing an antimicrobial peptide an/or analog and/or derivative of the present invention in a mammary gland or cell or tissue thereof, said method comprising introducing an expression construct or expression vector as described according to any embodiment hereof into a non-human mammal cell and regenerating or otherwise producing a non-human mammal there from. Preferably the nucleic acid construct is introduced into the genome of a pronucleus of a fertilized oocyte and the oocyte is maintained under conditions sufficient for an animal to be regenerated there from. Alternatively, the cell is a stem cell and said stem cell is maintained under conditions sufficient to regenerate a non-human mammal, e.g., introduced into a non-human mammal blastocyst, which is in turn introduced into a pregnant or pseudo-pregnant non-human mammal and permitted to develop into a non-human mammal. Alternatively, the cell is a somatic cell and the nucleus of said cell or said cell is introduced into an oocyte under conditions for de-differentiation to occur and the resulting cell maintained under conditions sufficient for an embryo to develop, which is in turn introduced into a pregnant or pseudo-pregnant non-human mammal and permitted to develop into a non-human mammal.

In another example, the present invention provides a method for producing a genetically-modified non-human mammal expressing an antimicrobial peptide and/or analog and/or derivative as described according to any embodiment hereof in a mammary gland or cell or tissue thereof, said method comprising:

(i) providing or obtaining an oocyte or sperm cell comprising an expression construct or expression vector as described according to any embodiment hereof; and
(ii) fertilizing said oocyte with a sperm cell or fertilizing an oocyte with said sperm cell to thereby produce a zygote and maintaining the zygote under conditions sufficient for development of a genetically-modified non-human mammal expressing an antimicrobial peptide and/or analog and/or derivative as described according to any embodiment hereof in a mammary gland or cell or tissue thereof,
thereby producing a genetically-modified non-human mammal expressing the antimicrobial peptide and/or analog and/or derivative as described according to any embodiment hereof in a mammary gland or cell or tissue thereof.

For example, the zygote is maintained under conditions sufficient for an embryo to form and the embryo administered or implanted into a uterus of a non-human mammal and permitted to develop into a genetically-modified non-human mammal.

In another example, the present invention provides a method for producing a genetically-modified non-human mammal expressing an antimicrobial peptide and/or analog and/or derivative as described according to any embodiment hereof in a mammary gland or cell or tissue thereof, said method comprising:

(i) providing or obtaining a zygote or embryo comprising an expression construct or expression vector as described according to any embodiment hereof; and
(ii) maintaining the zygote or embryo under conditions sufficient for development of a genetically-modified non-human mammal expressing an antimicrobial peptide and/or analog and/or derivative as described according to any embodiment hereof in a mammary gland or cell or tissue thereof,
thereby producing genetically-modified non-human mammal expressing the antimicrobial peptide and/or analog and/or derivative as described according to any embodiment hereof in a mammary gland or cell or tissue thereof.

In another example, the present invention additionally provides a method for producing a genetically-modified non-human mammal expressing an antimicrobial peptide and/or analog and/or derivative as described according to any embodiment hereof in a mammary gland or cell or tissue thereof, said method comprising:

(i) providing or obtaining a genetically-modified non-human mammal as described according to any embodiment hereof;
(ii) mating the genetically-modified non-human mammal at (i) with a non-human mammal of the same species to thereby produce offspring of the genetically-modified non-human mammal; and
(iii) identifying or selecting an offspring at (ii) comprising an expression construct or expression vector of the present invention,
thereby producing a genetically-modified non-human mammal expressing the antimicrobial peptide and/or analog and/or derivative as described according to any embodiment hereof in a mammary gland or cell or tissue thereof.

The method may additionally comprises identifying or selecting an offspring at (ii) and/or (iii) that expresses the antimicrobial peptide and/or analog and/or derivative as described according to any embodiment hereof in a mammary gland or cell or tissue thereof

As will be apparent from the foregoing, the present invention also provides for use of an expression construct or expression vector or genetically-modified non-human mammal as described according to any embodiment hereof to produce a genetically modified non-human mammal expressing an antimicrobial peptide and/or analog and/or derivative thereof as described according to any embodiment hereof in a mammary gland or cell or tissue thereof.

In another example, the antimicrobial peptides and/or analogs and/or complex and/or expression constructs and/or expression vectors are useful for preventing or treating bacterial infection in a human or non-human mammal.

In one example, the present invention provides a method for treating or preventing mastitis in a mammal e.g., a non-human mammal, said method comprising administering to mammal in need of treatment or prophylaxis a peptide and/or analog and/or derivative and/or complex and/or expression construct and/or expression vector of the present invention. Preferably the peptide and/or analog and/or derivative and/or complex and/or expression construct and/or expression vector of the present invention is administered for a time and under conditions sufficient to kill and/or prevent growth of S. uberis and, optionally, a microorganism selected from S. aureus, S. agalactiae and E. coli and combinations thereof in a mammary gland or cell or tissue thereof.

In one example, a subject in need of treatment suffers from mastitis and/or is infected with S. uberis and/or S. aureus and/or S. agalactiae and/or E. coli. For example, the subject has an increased number of somatic cells e.g., polymorphonuclear neutrophils (PMN) in a mammary gland or secretion thereof e.g., milk, and/or has a red, swollen mammary gland and/or flakes or clots (protein aggregates) in milk compared to a normal and/or healthy subject.

In one example, a subject in need of treatment or prophylaxis is a mammalian subject at risk of developing mastitis, e.g., a mammal in a prepartum period, e.g., in a non-human mammal that is not lactating and/or a lactating human female or other pregnant or lactating mammalian subject.

In a preferred example, the present invention provides a method for treating or preventing mastitis in a mammal e.g., a non-human mammal, said method comprising expressing in a mammary gland or a cell or tissue thereof a peptide and/or analog and/or derivative of the present invention in the mammalian subject in need of treatment or prophylaxis. For example, the method comprises administering an expression construct or expression vector of the present invention to a mammary gland or cell or tissue of the subject to thereby express the peptide and/or analog and/or derivative of the present invention. Suitable methods of administration of an expression construct or expression vector will be apparent to the skilled artisan and/or described herein. For example, the expression construct or expression vector is administered by high-pressure jet injection, or a virus, e.g., an adenovirus, comprising an expression construct is administered to a mammary gland of the subject or a or cell or tissue thereof.

In one example, a peptide and/or analog and/or derivative of the present invention is expressed in a non-human mammal by producing or obtaining a genetically-modified non-human mammal as described according to any embodiment hereof, wherein said genetically-modified non-human mammal expresses the antimicrobial peptide or analog or derivative of the present embodiment in a mammary gland or cell or tissue thereof. For example, the genetically modified non-human mammal is produced or obtained by performing a method described according to any embodiment hereof.

Preferably the genetically-modified non-human mammal expresses an amount of an antimicrobial peptide or analog or derivative sufficient to kill and/or prevent growth of S. uberis and, optionally a microorganism selected from S. aureus, S. agalactiae, E. coli and combinations thereof in a mammary gland or a cell or tissue thereof.

In one example, the antimicrobial activity of the antimicrobial peptide and/or analog and/or derivative is reduced or antagonized or partially or completely inhibited when contacted with milk, and said peptide and/or analog and/or derivative is expressed in a cell prior to lactation. For example, nucleic acid encoding such a peptide is operably-linked to a lactalbumin gene promoter or a lactoferrin gene promoter, which confer expression in a mammary gland or cell or tissue thereof prior to lactation. Expression of such a peptide is useful e.g., to prevent mastitis and/or prevent infection by S. uberis and/or S. aureus and/or S. agalactiae and/or E. coli prior to commencement of a first lactation. For example, the peptide comprises a sequence set forth in SEQ ID NO: 8.

In another example, the antimicrobial peptide and/or analog and/or derivative retains its activity in milk and/or a milk product and/or other dairy product e.g., cheese starter culture, yoghurt starter culture, cheese or yoghurt.

Expression of such a peptide prior to lactation and/or during lactation is useful for preventing or treating mastitis and/or treating or preventing infection by S. uberis and/or S. aureus and/or S. agalactiae and/or E. coli. Such a peptide can also be secreted into milk to treat or prevent mastitis and/or said infection. For example, the peptide comprises a sequence set forth in SEQ ID NO: 7.

In another example, the present invention also provides for use of an expression construct or expression vector or antimicrobial peptide and/or analog and/or derivative as described according to any embodiment hereof to treat or prevent mastitis and/or to treat or prevent an infection in a mammary gland or cell or tissue thereof by S. uberis and/or S. aureus and/or S. agalactiae and/or E. coli.

In another example, the present invention also provides for use of an expression construct or expression vector or antimicrobial peptide and/or analog and/or derivative as described according to any embodiment hereof in the manufacture of a medicament to treat or prevent mastitis and/or to treat or prevent an infection in a mammary gland or cell or tissue thereof by S. uberis and/or S. aureus and/or S. agalactiae and/or E. coli.

As will be apparent to the skilled artisan based on the description herein, mastitis is associated with reduced milk production in mammals. Because the present invention provides methods for treating or preventing mastitis, the present invention also provides methods for improving milk production in a non-human mammal. Accordingly, the present invention also provides a process for improving milk production in a non-human mammal, said process comprising performing a method as described according to any embodiment hereof to prevent or treat mastitis in a non-human mammal thereby improving milk production in the genetically-modified non-human mammal.

In another example, the present invention further provides a process for improving production of a recombinant polypeptide in milk of a genetically-modified non-human mammal, said process comprising performing a method as described according to any embodiment hereof to prevent or treat mastitis in a genetically-modified non-human mammal, wherein said genetically-modified non-human mammal secretes a recombinant peptide or polypeptide into milk produced from its mammary gland(s), thereby improving production of the recombinant polypeptide in milk of a genetically-modified non-human mammal. In this respect, the recombinant peptide or polypeptide can be an antimicrobial peptide or analog or derivative of the present invention, or any other peptide or polypeptide.

In another example, the present invention also provides a process for producing an antimicrobial peptide and/or analog and/or derivative as described according to any embodiment hereof, said process comprising:

(i) producing or obtaining a genetically-modified non-human mammal as described according to any embodiment hereof or producing a genetically-modified non-human mammal by performing a method as described according to any embodiment hereof;
(ii) maintaining the genetically-modified non-human mammal for a time and under conditions sufficient for the antimicrobial peptide and/or analog and/or derivative to be expressed,
thereby producing the antimicrobial peptide and/or analog and/or derivative.

Preferably, the process comprises obtaining or producing a genetically-modified non-human mammal that secretes the antimicrobial peptide and/or analog and/or derivative into milk, and maintaining the genetically-modified non-human mammal for a time and under conditions sufficient for lactation to occur to thereby produce milk comprising said antimicrobial peptide and/or analog and/or derivative.

In another example, the process additionally comprises isolating the antimicrobial peptide and/or derivative and/or analog e.g., from milk. Methods for isolating the antimicrobial peptide and/or analog and/or derivative will be apparent to the skilled artisan and/or described herein.

In another example, the present invention also provides for use of an expression construct or expression vector or genetically-modified non-human mammal as described according to any embodiment hereof to produce the antimicrobial peptide and/or derivative and/or analog of the present invention.

In another example, the present invention provides a method of producing a dairy product e.g., a fermented dairy product comprising providing an antimicrobial peptide, analog or derivative as described according to any embodiment hereof to a fermentation culture comprising one or more lactobacilli against which the peptide, analog or derivative has low activity to thereby prevent contamination or infection by a microbe against which the peptide has significant antimicrobial activity. The dairy product may be any dairy product comprising lactobacilli or for which lactobacilli are, used in production, e.g., cheese starter culture, yoghurt starter culture, cheese or yoghurt. Other dairy products are not excluded.

As will be apparent to the skilled artisan, an antimicrobial peptide and/or analog and/or derivative as described according to any embodiment hereof and/or produced by performing a method as described according to any embodiment hereof and/or milk comprising an antimicrobial peptide having bioactivity therein is useful for a variety of purposes in addition to treatment or prevention of mastitis, e.g., to treat or prevent microbial growth and/or to treat or prevent and infection and/or to prolong storage life of a perishable product. Suitable uses for the antimicrobial peptide and/or analog and/or derivative will be apparent to the skilled artisan based on the description herein.

In another example, the present invention provides a method for treating or preventing respiratory disease e.g., BRD or SRD, in a human or non-human mammal e.g., a bovine or porcine animal, said method comprising administering to mammal in need of treatment or prophylaxis a peptide and/or analog and/or derivative and/or complex and/or expression construct and/or expression vector of the present invention. Preferably the peptide and/or analog and/or derivative and/or complex and/or expression construct and/or expression vector of the present invention is administered for a time and under conditions sufficient to kill and/or prevent growth of E. coli and/or S. suis and/or P. aeruginosa and/or Mannheimia haemolytica (P. haemolytica) and/or P. multocida and/or A. pleuropneumoniae (APP) and/or H. somnus and/or Salmonella choleraesuis and/or B. bronchiseptica and combinations thereof, including one or two or three or four or five or six or seven or eight or nine of said organisms.

In another example, the present invention provides a method for treating or preventing clostridial intestinal disease in a human or non-human mammal e.g., an immune-compromized subject or subject receiving anti-TNF therapy, said method comprising administering to mammal in need of treatment or prophylaxis a peptide and/or analog and/or derivative and/or complex and/or expression construct and/or expression vector of the present invention. Preferably the peptide and/or analog and/or derivative and/or complex and/or expression construct and/or expression vector of the present invention is administered for a time and under conditions sufficient to kill and/or prevent growth of at least one bacterium of Clostridium spp., e.g., C. difficile and/or C. perfringens and combinations thereof.

In another example, the present invention provides a method for treating or preventing otitis externa in a human or non-human mammal e.g., a domestic animal such as a feline or canine, said method comprising administering to mammal in need of treatment or prophylaxis a peptide and/or analog and/or derivative and/or complex and/or expression construct and/or expression vector of the present invention. Preferably the peptide and/or analog and/or derivative and/or complex and/or expression construct and/or expression vector of the present invention is administered for a time and under conditions sufficient to kill and/or prevent growth of S. aureus and/or S. schleifen subsp. coagulans and/or S. schleiferi and/or S. epidermis and/or S. pseudointermedin and/or P. aeruginosa and any combinations thereof.

### DEFINITIONS

This specification contains nucleotide and amino acid sequence information prepared using PatentIn Version 3.4, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (e.g. SEQ ID NO: 1 refers to the sequence in the sequence listing designated as <400>1).

The designation of nucleotide residues referred to herein are those recommended by the IUPAC-TUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

### BRIEF DESCRIPTION OF THE DRAWINGS

FIG. 1a is a graphical representation of the minimum inhibitory concentration (MIC) of an antimicrobial peptide comprising an amino acid sequence set forth in SEQ ID NO: 7 as determined using a radial diffusion assay. The MIC (μg/ml) is indicated on the X-axis. The MIC is defined as the χ intercept of the least mean square regression lines through the respective data points. Results are means±standard error of the mean (SEM) from two experiments. The microorganism being tested is indicated on the Y axis.

FIG. 1b is a graphical representation of the minimum inhibitory concentration (MIC) of an antimicrobial peptide comprising an amino acid sequence set forth in SEQ ID NO: 8 as determined using a radial diffusion assay. The MIC (μg/ml) is indicated on the X-axis. The MIC is defined as the χ intercept of the least mean square regression lines through the respective data points. Results are means±standard error of the mean (SEM) from two experiments. The microorganism being tested is indicated on the Y axis.

FIG. 2a is a schematic representation showing the alignment of degenerate overlapping oligonucleotides (SEQ ID Nos: 113-122) to the sequences of AGG01 (Cath3; SEQ ID NO: 7) and AGG02 (Cath 4; SEQ ID NO: 8) peptides, showing the positions of variable residues as cross-bars. The schematic shows how the positions of variable sequences are conserved in the alignment overlapping oligonucleotides used for producing assembled mutated peptides as described in Example 2 hereof. Sequences of the oligonucleotides are presented below the aligned sequences.

FIG. 2b is a schematic representation showing the alignment of degenerate overlapping oligonucleotides (SEQ ID Nos: 123-128) to the sequences of AGG01 (Cath3; SEQ ID NO: 7) and AGG02 (Cath 4; SEQ ID NO: 8) peptides, showing the positions of variable residues as cross-bars. The schematic shows how the positions of variable sequences are conserved in the alignment overlapping oligonucleotides used for producing assembled mutated peptides as described in Example 2 hereof. Sequences of the oligonucleotides are presented below the aligned sequences.

FIG. 3a provides a graphical representation showing growth inhibition of antimicrobial peptides produced as described in FIGS. 2a and 2b (Example 2) over a time course of up to about 4 hours. Peptides are shown to the right. Growth was determined by optical density measurement at 600 nm. Data demonstrate that peptides designated A12, 5, 6 and 13 have enhanced growth inhibition activity compared to SEQ ID NO: 7 (AGG01) or SEQ ID NO: 8 (AGG02), or calmodulin and vector controls.

FIG. 3b provides a graphical representation showing growth inhibition of antimicrobial peptides produced as described in FIGS. 2a and 2b (Example 2) over a time course of up to about 4 hours. Peptides are shown to the right. Growth was determined by optical density measurement at 600 nm. Data demonstrate that peptides designated B6, E3, F7 and G9 have enhanced growth inhibition activity compared to SEQ ID NO: 7 (AGG01) or SEQ ID NO: 8 (AGG02).

### DETAILED DESCRIPTION OF THE PREFERRED EMBODIMENTS

### Peptides, Derivatives and Analogs

In one example, the present invention provides an antimicrobial peptide encoded by a nucleic acid comprising a sequence set forth in SEQ ID NO: 5 or an analog of said peptide or a derivative of said peptide. Preferably said peptide has antimicrobial activity against S. uberis and optionally, one or more microorganisms selected from S. aureus, or S. agalactiae and/or E. coli. Suitable antimicrobial peptides will be apparent to the skilled artisan based on the description herein.

As disclosed herein above, preferred antimicrobial peptides have a sequence having a degree of percentage identity to a reference sequence. In determining whether or not two amino acid sequences fall within the defined percentage identity limits supra, those skilled in the art will be aware that it is possible to conduct a side-by-side comparison of the amino acid sequences. In such comparisons or alignments, differences will arise in the positioning of non-identical residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using software of the Computer Genetics Group, Inc., University Research Park, Madison, Wis., United States of America, e.g., using the GAP program of Devereaux et al., Nucl. Acids Res. 12, 387-395, 1984, which utilizes the algorithm of Needleman and Wunsch, J. Mol. Biol. 48, 443-453, 1970. Alternatively, the CLUSTAL W algorithm of Thompson et al., Nucl. Acids Res. 22, 4673-4680, 1994, is used to obtain an alignment of multiple sequences, wherein it is necessary or desirable to maximize the number of identical/similar residues and to minimize the number and/or length of sequence gaps in the alignment.

Alternatively, a suite of commonly used and freely available sequence comparison algorithms is provided by the National Center for Biotechnology Information (NCBI) Basic Local Alignment Search Tool (BLAST) (Altschul et al. J. Mol. Biol. 215: 403-410, 1990), which is available from several sources, including the NCBI, Bethesda, Md. The BLAST software suite includes various sequence analysis programs including “blastn,” that is used to align a known nucleotide sequence with other polynucleotide sequences from a variety of databases and “blastp” used to align a known amino acid sequence with one or more sequences from one or more databases. Also available is a tool called “BLAST 2 Sequences” that is used for direct pairwise comparison of two nucleotide sequences.

As used herein the term “NCBI” shall be taken to mean the database of the National Center for Biotechnology Information at the National Library of Medicine at the National Institutes of Health of the Government of the United States of America, Bethesda, Md., 20894.

In this respect, non-natural amino acids shall be considered to be identical to their natural counterparts. Accordingly, a peptide comprising only non-natural amino acids (e.g., D-amino acids) equivalent to those set forth in SEQ ID NO: 7 shall be considered to have an amino acid sequence 100% identical to SEQ ID NO: 7.

Preferably an antimicrobial peptide or analog or derivative thereof is between about 6 to about 100 residues long (or any value there between), preferably from about 15 to 75 residues (or any value there between), preferably from about 20 to about 50 residues (or any value there between), and even more preferably from about 24 to about 40 residues (or any value there between).

### Peptide Analogs

Suitable peptide analogs include, for example, an antimicrobial peptide comprising one or more conservative amino acid substitutions. A “conservative amino acid substitution” is one in which the amino acid residue is replaced with an amino acid residue having a similar side chain.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine).

The importance of the hydropathic amino acid index in conferring interactive biological function on a protein is generally understood in the art (Kyte & Doolittle, J. Mol. Biol. 157, 105-132, 1982). It is known that certain amino acids may be substituted for other amino acids having a similar hydropathic index or score and still retain a similar biological activity, for example, the ability to bind to a membrane of a microorganism and/or kill the microorganism. The hydropathic index of amino acids also may be considered in determining a conservative substitution that produces a functionally equivalent molecule. Each amino acid has been assigned a hydropathic index on the basis of their hydrophobicity and charge characteristics, as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making changes based upon the hydropathic index, the substitution of amino acids whose hydropathic indices are within +/−0.2 is preferred. More preferably the substitution will involve amino acids having hydropathic indices within +/−0.1, and more preferably within about +/−0.05.

It is also understood in the art that the substitution of like amino acids is made effectively on the basis of hydrophilicity. As detailed in U.S. Pat. No. 4,554,101, the following hydrophilicity values have been assigned to amino acid residues: arginine (+3.0); lysine (+3.0); aspartate (+3.0+/−0.1); glutamate (+3.0+/−0.1); serine (+0.3); asparagine (+0.2); glutamine (+0.2); glycine (0); threonine (−0.4); proline (−0.5+/−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, it is preferred to substitute amino acids having hydrophilicity values within about +/−0.2 of each other, more preferably within about +/−0.1, and even more preferably within about +/−0.05

The present invention also contemplates non-conservative amino acid changes. For example, of particular interest are substitutions of charged amino acids with another charged amino acid and with neutral or positively charged amino acids. The latter of these substitutions results in an antimicrobial peptide analog having reduced positive charge, thereby improving the characteristics of the antimicrobial peptide.

Additional preferred peptide analogs have reduced immunogenicity compared to an antimicrobial peptide of the invention. Alternatively, or in addition, a preferred peptide analog has enhanced stability compared to an antimicrobial peptide of the invention.

It also is contemplated that other sterically similar compounds may be formulated to mimic the key portions of the peptide structure. Such compounds, which may be termed peptidomimetics, may be used in the same manner as the peptides of the invention and hence are also analogs of a peptide of the invention. The generation of such an analog may be achieved by the techniques of modeling and chemical design known to those of skill in the art. It will be understood that all such sterically similar antimicrobial peptide analogs fall within the scope of the present invention.

Another method for determining the “equivalence” of modified peptides involves a functional approach. For example, a given peptide analog is tested for its antimicrobial activity e.g., using any screening method described herein.

Particularly preferred analogs of a peptide of the invention will comprise one or more non-naturally occurring amino acids or amino acid analogs. For example, an antimicrobial peptide analog of the invention comprises one or more naturally-occurring non-genetically encoded L-amino acids, synthetic L-amino acids or D-enantiomers of an amino acid. For example, the peptide comprises only D-amino acids. In another example, the analog comprises one or more residues selected from the group consisting of: hydroxyproline, β-alanine, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylananine 3-benzothienyl alanine 4-chlorophenylalanine, 2-fluorophenylalanine, 3-fluorophenylalanine, 4-fluorophenylalanine, penicillamine, 1,2,3,4-tetrahydrotic isoquinoline-3-carboxylic acid β-2-thienylalanine, methionine sulfoxide, homoarginine, N-acetyl lysine, 2,4-diamino butyric acid, p-aminophenylalanine, N-methylvaline, homocysteine, homoserine, ε-amino hexanoic acid, δ-amino valeric acid, 2,3-diaminobutyric acid and mixtures thereof.

Commonly-encountered amino acids that are not genetically encoded and which can be present, or substituted for an amino acid in an analog of an antimicrobial peptide of the invention include, but are not limited to, β-alanine (β-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); δ-aminovaleric acid (Ava); methylglycine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4—Cl)); 2-fluorophenylalanine (Phe(2—F)); 3-fluorophenylalanine (Phe(3—F)); 4-fluorophenylalanine (Phe(4—F)); penicillamine (Pen); 1,2,3,4-tetrahydroisoquinoline-3-carboxylic acid (Tic); .beta.-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH₂)); N-methyl valine (MeVal); homocysteine (hCys) and homoserine (hSer).

Other amino acid residues that are useful for making the peptides and peptide analogs described herein can be found, e.g., in Fasman, 1989, CRC Practical Handbook of Biochemistry and Molecular Biology, CRC Press, Inc., and the references cited therein.

The present invention additionally encompasses an isostere of a peptide described herein. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) known to those skilled in the art. Such modifications include modifications of the amide nitrogen, the α-carbon, amide carbonyl, complete replacement of the amide bond, extensions, deletions or backbone crosslinks. Several peptide backbone modifications are known, including ψ[CH₂S], ψ[CH₂NH], ψ[CSNH₂], ψ[NHCO], ψ[COCH₂], and ψ[(E) or (Z) CH═CH]. In the nomenclature used above, w indicates the absence of an amide bond. The structure that replaces the amide group is specified within the brackets.

Other modifications include, for example, an N-alkyl (or aryl) substitution (ψ[CONR]), or backbone cross-linking to construct lactams and other cyclic structures. Other derivatives of the modulator compounds of the invention include C-terminal hydroxymethyl derivatives, O-modified derivatives (e.g., C-terminal hydroxymethyl benzyl ether), N-terminally modified derivatives including substituted amides such as alkylamides and hydrazides.

In another embodiment, the peptide analog is a retro peptide analog (Goodman et al., Accounts of Chemical Research, 12:1-7, 1979). A retro peptide analog comprises a reversed amino acid sequence of an antimicrobial peptide of the present invention. For example, a retro peptide analog of an antimicrobial peptide of the present comprises an amino acid sequence set forth in any one of SEQ ID NOs: 33-58.

In a preferred embodiment, an analog of an antimicrobial peptide of the invention is a retro-inverted peptide (Sela and Zisman, FASEB J. 11:449, 1997). Evolution has ensured the almost exclusive occurrence of L-amino acids in naturally occurring proteins. As a consequence, virtually all proteases cleave peptide bonds between adjacent L-amino acids. Accordingly, artificial proteins or peptides composed of D-amino acids are preferably resistant to proteolytic breakdown. Retro-inverted peptide analogs are isomers of linear peptides in which the direction of the amino acid sequence is reversed (retro) and the chirality, D- or L-, of one or more amino acids therein is inverted (inverso) e.g., using D-amino acids rather than L-amino acids, e.g., Jameson et al., Nature, 368, 744-746 (1994); Brady et al., Nature, 368, 692-693 (1994). The net result of combining D-enantiomers and reverse synthesis is that the positions of carbonyl and amino groups in each amide bond are exchanged, while the position of the side-chain groups at each alpha carbon is preserved.

An advantage of retro-inverted peptides is their enhanced activity in vivo due to improved resistance to proteolytic degradation, i.e., the peptide has enhanced stability. (e.g., Chorev et al., Trends Biotech. 13, 438-445, 1995).

Retro-inverted peptide analogs may be complete or partial. Complete retro-inverted peptides are those in which a complete sequence of an antimicrobial peptide of the invention is reversed and the chirality of each amino acid in a sequence is inverted e.g., a peptide comprising an amino acid sequence set forth in any one of SEQ ID Nos:59-83. Partial retro-inverted peptide analogs are those in which some or all of the peptide bonds are reversed (i.e., completely reversed sequence) and the chirality of some, but not all, amino acid residues is inverted. Partial retro-inverted peptide analogs can also have only some of the peptide bonds are reversed and the chirality of only those amino acid residues in the reversed portion inverted. For example, one or two or three or four or five or six or seven or eight or nine or ten or eleven or twelve or thirteen or fourteen or fifteen or sixteen or seventeen or eighteen or nineteen or twenty or twenty one or twenty two or twenty three or twenty four or twenty five or twenty six or twenty seven or twenty eight or twenty nine or thirty or thirty one or thirty two or thirty three or thirty four or thirty five or thirty six or thirty seven or thirty eight amino acid residues are D-amino acids. The present invention clearly encompasses both partial and complete retro-inverted peptide analogs.

In another example, an analog of a peptide is modified to reduce the immunogenicity of said analog. Such reduced immunogenicity is useful for a peptide that is to be injected into a subject. Methods for reducing the immunogenicity of a peptide will be apparent to the skilled artisan. For example, an antigenic region of a peptide is predicted using a method known in the art and described, for example, in Kolaskar and Tongaonkar FEBS Letters, 276: 172-174, 1990. Any identified antigenic region may then be modified to reduce the immunogenicity of a peptide analog, provided that said analog is an antimicrobial peptide analog.

### Peptide Derivatives

Preferred derivatives include, for example, a fragment or processed form of an antimicrobial peptide of the invention. Preferred derivatives have reduced immunogenicity. For example, by deleting an antigenic determinant from an antimicrobial peptide of the invention, a derivative is produced having reduced immunogenicity.

Alternatively, or in addition, a preferred derivative of an antimicrobial peptide of the invention has enhanced antimicrobial activity.

Alternatively, or in addition, a preferred derivative of an antimicrobial peptide of the invention has enhanced stability. For example, a cleavage site of a protease active in a subject to which a peptide is to be administered is mutated and/or deleted to produce a stable derivative of an antimicrobial peptide of the invention.

Methods for producing additional derivatives of an antimicrobial peptide of the invention will be apparent to the skilled artisan and include recombinant methods, e.g., as exemplified herein. For example, the sequences encoding two antimicrobial peptides are aligned, and a consensus sequence produced that is capable of encoding either peptide. Such a consensus sequence is capable of encoding an amino acid that occurs at each position in either peptide, in addition to encoding amino acids that do not occur in either peptide. In this manner peptides that are hybrids of both base peptides are produced.

In another example, a nucleic acid encoding an antimicrobial peptide of the invention or an analog thereof is amplified using mutagenic PCR and the resulting nucleic acid expressed to produce a peptide using a method known in the art and/or described herein.

In a preferred embodiment, the nucleic acid fragments are modified by amplifying a nucleic acid fragment using mutagenic PCR. Such methods include a process selected from the group consisting of: (i) performing the PCR reaction in the presence of manganese; and (ii) performing the PCR in the presence of a concentration of dNTPs sufficient to result in mis-incorporation of nucleotides.

Methods of inducing random mutations using PCR are known in the art and are described, for example, in Dieffenbach (ed) and Dveksler (ed) (In: PCR Primer: A Laboratory Manual, Cold Spring Harbour Laboratories, NY, 1995). Furthermore, commercially available kits for use in mutagenic PCR are obtainable, such as, for example, the Diversify PCR Random Mutagenesis Kit (Clontech) or the GeneMorph Random Mutagenesis Kit (Stratagene).

Peptide derivatives of the present invention also encompass an antimicrobial peptide or an analog thereof as described herein in any embodiment that is modified to contain one or more-chemical moieties other than an amino acid. The chemical moiety may be linked covalently to the peptide or analog e.g., via an amino terminal amino acid residue, a carboxy terminal amino acid residue, or at an internal amino acid residue. Such modifications include the addition of a protective or capping group on a reactive moiety in the peptide, addition of a detectable label, and other changes that do not adversely destroy the activity of the peptide compound (e.g., its antimicrobial activity).

An “amino-terminal capping group” of a peptide derivative described herein is any chemical compound or moiety that is covalently linked to or conjugated to the amino terminal amino acid residue of a peptide or analog. An amino-terminal capping group may be useful to inhibit or prevent intramolecular cyclization or intermolecular polymerization, to protect the amino terminus from an undesirable reaction with other molecules, or to provide a combination of these properties. A peptide derivative of this invention that possesses an amino-terminal capping group may possess other beneficial activities as compared with the uncapped peptide, such as enhanced efficacy or reduced side effects. Examples of amino terminal capping groups that are useful in preparing peptide derivatives according to the invention include, but are not limited to, 1 to 6 naturally occurring L-amino acid residues, preferably 1-6 lysine residues, 1-6 arginine residues, or a combination of lysine and arginine residues; urethanes; urea compounds; lipoic acid (“Lip”); glucose-3-O-glycolic acid moiety (“Gga”); or an acyl group that is covalently linked to the amino terminal amino acid residue of a peptide, wherein such acyl groups useful in the compositions of the invention may have a carbonyl group and a hydrocarbon chain that ranges from one carbon atom (e.g., as in an acetyl moiety) to up to 25 carbons (e.g., palmitoyl group, “Palm” (16:0) and docosahexaenoyl group, “DHA” (C22:6-3)). Furthermore, the carbon chain of the acyl group may be saturated, as in Palm, or unsaturated, as in DHA. It is understood that when an acid, such as docosahexaenoic acid, palmitic acid, or lipoic acid is designated as an amino terminal capping group, the resultant peptide compound is the condensed product of the uncapped peptide and the acid.

A “carboxy-terminal capping group” of a peptide derivative described herein is any chemical compound or moiety that is covalently linked or conjugated to the carboxy terminal amino acid residue of a peptide or analog. The primary purpose of such a carboxy-terminal capping group is to inhibit or prevent intramolecular cyclization or intermolecular polymerization or to provide a combination of these properties. A peptide derivative of this invention possessing a carboxy-terminal capping group may also possess other beneficial activities as compared with an uncapped peptide, such as enhanced efficacy, reduced side effects, enhanced hydrophilicity, enhanced hydrophobicity. Carboxy-terminal capping groups that are particularly useful in the peptide derivatives described herein include primary or secondary amines that are linked by an amide bond to the α-carboxyl group of the carboxy terminal amino acid of the peptide compound. Other carboxy terminal capping groups useful in the invention include aliphatic primary and secondary alcohols and aromatic phenolic derivatives, including flavenoids, with 1 to 26 carbon atoms, which form esters when linked to the carboxylic acid group of the carboxy-terminal amino acid residue of a peptide described herein.

Other chemical modifications of a peptide or analog, include, for example, glycosylation, acetylation (including N-terminal acetylation), carboxylation, carbonylation, phosphorylation, PEGylation, amidation, addition of trans olefin, substitution of α-hydrogens with methyl groups, derivatization by known protecting/blocking groups, circularization, linkage to an antibody or other cellular ligand, etc. Any of numerous chemical modifications may be carried out by known techniques, including but not limited to specific chemical cleavage by cyanogen bromide, trypsin, chymotrypsin, papain, V8 protease, NaBH₄, acetylation, formylation, oxidation, reduction, etc.

### Fusion Proteins and Complexes

The present invention provides an additional derivative of an antimicrobial peptide of the invention, such as, for example a fusion protein comprising one or more of the antimicrobial peptides and/or analogs of the invention. For example, the antimicrobial peptide or analog is fused to a tag or label. Such a tag or label facilitates purification or isolation of the antimicrobial peptide and/or analog and/or derivative or detection of the peptide, analog or derivative. Suitable tags will be apparent to the skilled artisan and include, for example, influenza virus hemagglutinin (HA), Simian Virus 5 (V5), polyhistidine, c-myc or FLAG.

In another example, a fusion protein of the present invention comprises a plurality of antimicrobial peptides of the invention and/or analogs thereof. In this respect, the fusion protein may comprise multiple copies of the same antimicrobial peptide or analog and/or a plurality of antimicrobial peptides and/or analogs (whether present in a single copy or a plurality of copies).

In one embodiment, such a fusion protein comprises one or more additional components, such as, for example, a tag or label and/or an additional antimicrobial peptide or analog or derivative thereof.

In a further example, a fusion protein as described according to any embodiment hereof comprises an antimicrobial peptide or analog or derivative thereof fused to another protein or a fragment thereof so as to constrain and/or display said antimicrobial peptide or analog within said other protein. Polypeptides used for such purposes are capable of reducing the flexibility of another protein's amino and/or carboxyl termini. Preferably such proteins provide a rigid scaffold or platform for the protein. In addition, such proteins preferably are capable of providing protection from proteolytic degradation and the like, and/or are capable of enhancing solubility. In one example, the antimicrobial peptide or analog thereof is fused to a protein or fragment thereof that is expressed in a non-human mall in nature. In one example, the antimicrobial peptide or analog is fused to a naturally-occurring antimicrobial peptide, e.g., a peptide comprising a sequence set forth in any one of SEQ ID NOs: 104-111, or a fragment thereof, e.g., a prepro region of a sequence set forth in any one of SEQ ID NOs: 104-111.

Each of the components of a derivative of an antimicrobial peptide of the invention may optionally be separated by a linker that facilitates the independent folding of each of said components. A suitable linker will be apparent to the skilled artisan. For example, it is often unfavourable to have a linker sequence with high propensity to adopt α-helix or β-strand structures, which could limit the flexibility of the protein and consequently its functional activity. Rather, a more desirable linker is a sequence with a preference to adopt extended conformation. In practice, most currently designed linker sequences have a high content of glycine residues that force the linker to adopt loop conformation. Glycine is generally used in designed linkers because the absence of a β-carbon permits the polypeptide backbone to access dihedral angles that are energetically forbidden for other amino acids.

Preferably the linker is hydrophilic, i.e. the residues in the linker are hydrophilic.

Linkers comprising glycine and/or serine have a high freedom degree for linking of two proteins, i.e., they enable the fused proteins to fold and produce functional proteins. Robinson and Sauer Proc. Natl. Acad. Sci. 95: 5929-5934, 1998 found that it is the composition of a linker peptide that is important for stability and folding of a fusion protein rather than a specific sequence. For example, the authors found that a fusion protein comprising a linker consisting almost entirely of glycine was unstable. Accordingly, the use of amino acid residues other than glycine, such as, for example, alanine or serine, is also useful for the production of a linker.

In one embodiment, the linker is a glycine rich linker. Preferably the linker is a glycine linker that additionally comprises alanine and/or serine.

### Peptide Synthesis

In one example, an antimicrobial peptide of the invention or an analog or derivative thereof synthesized using a chemical method known to the skilled artisan. For example, synthetic peptides are prepared using known techniques of solid phase, liquid phase, or peptide condensation, or any combination thereof, and can include natural and/or unnatural amino acids Amino acids used for peptide synthesis may be standard Boc (Nα-amino protected Nα-t-butyloxycarbonyl)amino acid resin with the deprotecting, neutralization, coupling and wash protocols of the original solid phase procedure of Merrifield, J. Am. Chem. Soc., 85:2149-2154, 1963, or the base-labile Nα-amino protected 9-fluorenylmethoxycarbonyl (Fmoc) amino acids described by Carpino and Han, J. Org. Chem., 37:3403-3409, 1972. Both Fmoc and Boc Nα-amino protected amino acids can be obtained from various commercial sources, such as, for example, Fluka, Bachem, Advanced Chemtech, Sigma, Cambridge Research Biochemical, Bachem, or Peninsula Labs.

Generally, chemical synthesis methods comprise the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis. Synthesis. Biology, Vol. 1, for classical solution synthesis. These methods are suitable for synthesis of an antimicrobial peptide of the present invention or an analog or derivative thereof.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobomyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

The antimicrobial peptide, analog or derivative of the present invention can also be chemically prepared by other methods such as by the method of simultaneous multiple peptide synthesis. See, e.g., Houghten Proc. Natl. Acad. Sci. USA 82: 5131-5135, 1985 or U.S. Pat. No. 4,631,211.

As will be apparent to the skilled artisan based on the description herein, an analog or derivative of an antimicrobial of the invention may comprise D-amino acids, a combination of D- and L-amino acids, and various unnatural amino acids (e.g., α-methyl amino acids, Cα-methyl amino acids, and Nα-methyl amino acids, etc) to convey special properties. Synthetic amino acids include ornithine for lysine, fluorophenylalanine for phenylalanine, and norleucine for leucine or isoleucine. Methods for the synthesis of such peptides will be apparent to the skilled artisan based on the foregoing.

### Recombinant Peptide Production

In another example, an antimicrobial peptide or analog or derivative thereof is produced as a recombinant protein. To facilitate the production of a recombinant peptide or fusion protein nucleic acid encoding same is preferably isolated or synthesized. Typically the nucleic acid encoding the constituent components of the fusion protein is/are isolated using a known method, such as, for example, amplification (e.g., using PCR or splice overlap extension) or isolated from nucleic acid from an organism using one or more restriction enzymes or isolated from a library of nucleic acids. Methods for such isolation will be apparent to the ordinary skilled artisan and/or described in Ausubel et al (In: Current Protocols in Molecular Biology. Wiley Interscience, ISBN 047 150338, 1987), Sambrook et al (In: Molecular Cloning: Molecular Cloning: A Laboratory Manual, Cold Spring Harbor Laboratories, New York, Third Edition 2001).

For example, nucleic acid (e.g., genomic DNA or RNA that is then reverse transcribed to form cDNA) from a cell or organism capable of expressing an antimicrobial peptide of the invention is isolated using a method known in the art and cloned into a suitable vector. The vector is then introduced into a suitable organism, such as, for example, a bacterial cell. Using a nucleic acid probe from a known antimicrobial peptide encoding gene a cell comprising the nucleic acid of interest is isolated using methods known in the art and described, for example, in Ausubel et al (In: Current Protocols in Molecular Biology. Wiley Interscience, ISBN 047 150338, 1987), Sambrook et al (In: Molecular Cloning: Molecular Cloning: A Laboratory Manual, Cold Spring Harbor Laboratories, New York, Third Edition 2001).

Alternatively, nucleic acid encoding an antimicrobial peptide of the invention is isolated using polymerase chain reaction (PCR). Methods of PCR are known in the art and described, for example, in Dieffenbach (ed) and Dveksler (ed) (In: PCR Primer: A Laboratory Manual, Cold Spring Harbour Laboratories, NY, 1995). Generally, for PCR two non-complementary nucleic acid primer molecules comprising at least about 20 nucleotides in length, and more preferably at least 25 nucleotides in length are hybridized to different strands of a nucleic acid template molecule, and specific nucleic acid molecule copies of the template are amplified enzymatically. Preferably the primers hybridize to nucleic acid adjacent to a nucleic acid encoding an antimicrobial peptide of the invention, thereby facilitating amplification of the nucleic acid that encodes the subunit. Following amplification, the amplified nucleic acid is isolated using a method known in the art and, preferably cloned into a suitable vector.

Other methods for the production of a nucleic acid of the invention will be apparent to the skilled artisan and are encompassed by the present invention.

For expressing protein by recombinant means, a protein-encoding nucleotide sequence is placed in operable connection with a promoter or other regulatory sequence capable of regulating expression in a cell-free system or cellular system. For example, nucleic acid comprising a sequence that encodes an antimicrobial peptide of the present invention in operable connection with a suitable promoter is expressed in a suitable cell for a time and under conditions sufficient for expression to occur. Nucleic acid encoding an antimicrobial protein of the present invention is readily derived from the publicly available amino acid sequence.

Should it be preferred that a peptide or fusion protein of the invention is expressed in vitro a suitable promoter includes, but is not limited to a T3 or a T7 bacteriophage promoter (Hanes and Plückthun Proc. Natl. Acad. Sci. USA, 94 4937-4942 1997).

Typical expression vectors for in vitro expression or cell-free expression have been described and include, but are not limited to the TNT T7 and TNT T3 systems (Promega), the pEXP1-DEST and pEXP2-DEST vectors (Invitrogen).

Typical promoters suitable for expression in bacterial cells include, but are not limited to, the lacz promoter, the Ipp promoter, temperature-sensitive λL or λR promoters, T7 promoter, T3 promoter, SP6 promoter or semi-artificial promoters such as the IPTG-inducible tac promoter or lacUV5 promoter. A number of other gene construct systems for expressing the nucleic acid fragment of the invention in bacterial cells are well-known in the art and are described for example, in Ausubel et al (In: Current Protocols in Molecular Biology. Wiley Interscience, ISBN 047 150338, 1987), U.S. Pat. No. 5,763,239 (Diversa Corporation) and Sambrook et al (In: Molecular Cloning: Molecular Cloning: A Laboratory Manual, Cold Spring Harbor Laboratories, New York, Third Edition 2001).

Numerous expression vectors for expression of recombinant polypeptides in bacterial cells and efficient ribosome binding sites have been described, and include, for example, PKC30 (Shimatake and Rosenberg, Nature 292, 128, 1981); pKK173-3 (Amann and Brosius, Gene 40, 183, 1985), pET-3 (Studier and Moffat, J. Mol. Biol. 189, 113, 1986); the pCR vector suite (Invitrogen), pGEM-T Easy vectors (Promega), the pL expression vector suite (Invitrogen) the pBAD/TOPO or pBAD/thio-TOPO series of vectors containing an arabinose-inducible promoter (Invitrogen, Carlsbad, Calif.), the latter of which is designed to also produce fusion proteins with a Trx loop for conformational constraint of the expressed protein; the pFLEX series of expression vectors (Pfizer Inc., CT, USA); the pQE series of expression vectors (QIAGEN, CA, USA), or the pL series of expression vectors (Invitrogen), amongst others.

Typical promoters suitable for expression in viruses of eukaryotic cells and eukaryotic cells include the SV40 late promoter, SV40 early promoter and cytomegalovirus (CMV) promoter, CMV IE (cytomegalovirus immediate early) promoter amongst others. Preferred vectors for expression in mammalian cells (e.g., 293, COS, CHO, 10T cells, 293T cells) include, but are not limited to, the pcDNA vector suite supplied by Invitrogen, in particular pcDNA 3.1 myc-His-tag comprising the CMV promoter and encoding a C-terminal 6×His and MYC tag; and the retrovirus vector pSRαtkneo (Muller et al., Mol. Cell. Biol., 11, 1785, 1991).

A wide range of additional host/vector systems suitable for expressing an antimicrobial peptide or fusion protein of the present invention are available publicly, and described, for example, in Sambrook et al (In: Molecular cloning, A laboratory manual, second edition, Cold Spring Harbor Laboratory, Cold Spring Harbor, N.Y., 1989).

Means for introducing the isolated nucleic acid molecule or a gene construct comprising same into a cell for expression are well-known to those skilled in the art. The technique used for a given organism depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

### Peptide/Analog/Derivative/Fusion Protein Isolation

Following production/expression/synthesis, an antimicrobial peptide of the invention or derivative or analog thereof is purified using a method known in the art. Such purification preferably provides a peptide of the invention substantially free of conspecific protein, acids, lipids, carbohydrates, and the like. Antibodies and other affinity ligands are particularly preferred for producing isolated protein. Preferably the protein will be in a preparation wherein more than about 90% (e.g. 95%, 98% or 99%) of the protein in the preparation is an antimicrobial peptide of the invention or derivative or analog thereof or fusion protein comprising same.

Standard methods of peptide purification are employed to obtain an isolated peptide of the invention, including but not limited to various high-pressure (or performance) liquid chromatography (HPLC) and non-HPLC peptide isolation protocols, such as size exclusion chromatography, ion exchange chromatography, phase separation methods, electrophoretic separations, precipitation methods, salting in/out methods, immunochromatography, and/or other methods.

A preferred method of isolating peptide compounds useful in compositions and methods of the invention employs reversed-phase HPLC using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can also be used to separate a peptide based on its charge.

Alternatively, affinity purification is useful for isolating a fusion protein comprising a label. Methods for isolating a protein using affinity chromatography are known in the art and described, for example, in Scopes (In: Protein purification: principles and practice, Third Edition, Springer Verlag, 1994). For example, an antibody or compound that binds to the label (in the case of a polyhistidine tag this may be, for example, nickel-NTA) is preferably immobilized on a solid support. A sample comprising a fusion protein is then contacted to the immobilized antibody or compound for a time and under conditions sufficient for binding to occur. Following washing to remove any unbound or non-specifically bound protein, the fusion protein is eluted.

The degree of purity of the peptide compound may be determined by various methods, including identification of a major large peak on HPLC. A peptide compound that produces a single peak that is at least 95% of the input material on an HPLC column is preferred. Even more preferable is a polypeptide that produces a single peak that is at least 97%, at least 98%, at least 99% or even 99.5% of the input material on an HPLC column.

To ensure that a peptide obtained using any of the techniques described above is the desired peptide for use in compositions and methods of the present invention, analysis of the composition of the peptide is determined by any of a variety of analytical methods known in the art. Such composition analysis may be conducted using high resolution mass spectrometry to determine the molecular weight of the peptide. Alternatively, the amino acid content of a peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine the sequence of the peptide. Since some of the peptide compounds contain amino and/or carboxy terminal capping groups, it may be necessary to remove the capping group or the capped amino acid residue prior to a sequence analysis. Thin-layer chromatographic methods may also be used to authenticate one or more constituent groups or residues of a desired peptide.

### Expression Constructs

### Nucleic Acid Encoding an Antimicrobial Peptide or Analog or Derivative

Nucleic acids that encode one or more antimicrobial peptides or analogs or derivatives of the present invention will be apparent to the skilled artisan based on the description herein.

For example, the nucleotide sequence of suitable nucleic acids encode an antimicrobial peptide or analog or derivative thereof individually or collectively selected from the group consisting of:

(i) a peptide comprising a sequence set forth in any one of SEQ ID NOs: 7-32;
(ii) an analog of (i) comprising a sequence set forth in any one of SEQ ID NOs: 33-83; and
(iii) a derivative of (i) having at least about 90% or 95% sequence identity thereto and comprising a sequence that differs from a sequence set forth in (i) or (ii) by one or more conservative amino acid substitutions, wherein said peptide has antimicrobial activity.

In determining whether or not two sequences fall within these defined percentage identity limits, those skilled in the art will be aware that it is possible to conduct a side-by-side comparison of the sequences. In such comparisons or alignments, differences will arise in the positioning of non-identical residues depending upon the algorithm used to perform the alignment. In the present context, references to percentage identities and similarities between two or more sequences shall be taken to refer to the number of identical and similar residues respectively, between said sequences as determined using any standard algorithm known to those skilled in the art. For example, nucleotide identities and similarities are calculated using software of the Computer Genetics Group, Inc., University Research Park, Maddison, Wis., United States of America, e.g., using the GAP program of Devereaux et al., Nucl. Acids Res. 12, 387-395, 1984, which utilizes the algorithm of Needleman and Wunsch, J. Mol. Biol. 48, 443-453, 1970. Alternatively, the CLUSTAL W algorithm of Thompson et al., Nucl. Acids Res. 22, 4673-4680, 1994, is used to obtain an alignment of multiple sequences, wherein it is necessary or desirable to maximize the number of identical/similar residues and to minimize the number and/or length of sequence gaps in the alignment. Sequence alignments can also be performed using a variety of other commercially available sequence analysis programs, such as, for example, the BLAST program available at NCBI.

Preferably the expression construct comprises a sequence encoding a peptide comprising a sequence set forth in any one of SEQ ID NOs: 7-32, more preferably any one of SEQ ID NOs: 10-13 and 23-32, still more preferably any one of SEQ ID NOs: 10-13, 24, 25, 28 or 30.

For example, the genetically-modified non-human mammal comprises an expression construct comprising a sequence set forth in any one of SEQ ID NOs: 84-88.90 or 91, preferably any one of SEQ ID Nos: 84-86 including SEQ ID NO: 84 or 85 or 86.

The expression construct can also optionally comprise a transcriptional terminator that is operative in the animal to which the construct is to be introduced. Furthermore, the gene construct may comprise a nucleic acid comprising the sequence of a polyadenylation signal operative in animal to which the construct is to be introduced.

### Promoters

Suitable promoters will be apparent to the skilled artisan. For example, a variety of transcriptional promoters that preferentially activate transcription in mammary epithelial cells are available. These include the promoters that control the genes encoding milk proteins such as caseins (αS1-, αS2-, β-, γ-, and κ-casein), β-lactoglobulin (Clark et al., 1989, Bio/Technology 7: 487-492), whey acid protein (Gordon et al., 1987, Bio/Technology 5: 1183-1187), and α-lactalbumin (Soulier et al., 1992, FEBS Letters 297: 13).

Nucleotide sequences of many promoters are publicly available, e.g., in GenBank and in scientific publications such as:

1) rat α-lactalbumin (Richards et al., 1981, J. Biol. Chem. 256: 526-532);

### 2) rat WAP (Campbell et al., 1984, Nucleic Acids Res. 12: 8685-8697);

3) rat α-casein (Jones et al., 1985, J. Biol. Chem. 260: 7042-7050);
4) rat α-casein (Lee and Rosen, 1983, J. Biol. Chem. 258: 10794-10804);
5) human alpha-lactalbumin (Hall, 1987, Biochem. J. 242: 735-742);
6) bovine α-S1 casein (Stewart, 1984, Nucleic Acids Res. 12: 389);
7) bovine α-casein (Gorodetsky et al., 1988, Gene 66: 87-96);
8) bovine α-casein (Alexander et al., 1988, Eur. J. Biochem. 178: 395-401);
9) bovine α-S2 casein (Brignon et al., 1977, FEBS Letters 188: 48-55);
10) bovine α-lactoglobulin (Jamieson et al., 1987, Gene 61: 85-90; Alexander et al., 1989, Nucleic Acids Res. 17: 6739).

In one example, a suitable promoters is a bovine β-casein gene promoter comprising a sequence set forth in SEQ ID NO: 92. In another example, a suitable promoter is a murine prolactin-inducible mammary specific promoter comprising a sequence set forth in SEQ ID NO: 93. In another example, a suitable promoter is a water buffalo α-lactalbumin gene promoter comprising a sequence set forth in SEQ ID NO: 94. In another example, a suitable promoter is a murine whey acidic protein (WAP) gene promoter comprising a sequence set forth in SEQ ID NO: 95 or a camel WAP gene promoter comprising a sequence set forth in SEQ ID NO: 96 or a rat WAP gene promoter comprising a sequence set forth in SEQ ID NO: 97. In another example, a suitable promoter is a caprine β-lactoglobulin gene promoter comprising a sequence set forth in SEQ ID NO: 98 or an ovine β-lactoglobulin gene promoter comprising a sequence set forth in SEQ ID NO: 99 or a caprine β-lactoglobulin gene promoter comprising a sequence set forth in SEQ ID NO: 100.

In another example, the promoter is a NF-κB promoter. For example, the promoter is derived from a lactoferrin gene. For example, the promoter comprises 4.4 kb of nucleic acid 5′ to a bovine lactoferrin gene, e.g., as described in Zheng et al., Gene, 353: 107-117, 2005.

Whilst it is preferable that a promoter used in an expression construct is derived from a non-human mammal into which the expression construct is to be introduced to increase the likelihood of correct expression, this is not essential. For example, rat and murine WAP gene promoters have been shown to be effective at inducing expression of proteins in other non-human mammals.

In the case of an expression construct to be administered to a mammary gland or cell or tissue thereof a suitable promoter also includes a promoter that expresses in a variety of cells in a non-human mammal including a mammary cell. For example, the promoter is a human cytomegalovirus (hCMV) immediate early (IE) promoter or a simian virus (SV)-40 promoter and/or enhancer.

### Signal Peptide Sequences

In one example, an expression construct encodes an antimicrobial peptide or analog or derivative fused to signal peptide, particularly a signal peptide of a milk specific gene, e.g., to induce or enhance secretion of an encoded peptide or analog or derivative into milk. For optimal secretion efficiency, the milk-specific signal peptide sequence is preferably derived from the same gene as the promoter used in the transgene.

Exemplary signal sequences are from genes coding for caseins, e.g., αS1-, αS2-, β-, γ-, and κ-casein, β-lactoglobulin, whey acid protein, or lactalbumin. For example, the sequence of a signal peptide from an α-1 lactalbumin is set forth in SEQ ID NO: 101. The sequence of a signal peptide from an αS1-casein protein is set forth in SEQ ID NO: 102. the sequence of a signal peptide from a β-lactoglobulin protein is set forth in SEQ ID NO: 103.

Selectable and/or Detectable Markers

In one embodiment, an expression construct comprises a nucleic acid encoding a detectable and/or selectable marker operably linked to a promoter. Such a marker facilitates the detection and/or selection of a genetically-modified cell or animal.

As used herein the term “selectable marker” shall be taken to mean a protein or peptide that confers a phenotype on a cell expressing said selectable marker that is not shown by those cells that do not carry said selectable marker. Examples of selectable markers include, but are not limited to the dhfr resistance gene, which confers resistance to methotrexate (Wigler, et al., Proc. Natl. Acad. Sci. USA 77:3567, 1980); the gpt resistance gene, which confers resistance to mycophenolic acid (Mulligan & Berg, Proc. Natl. Acad. Sci. USA 78:2072, 1981); the neomycin phosphotransferase gene, which confers resistance to the aminoglycoside G-418 (Colberre-Garapin, et al., J. Mol. Biol. 150:1, 1981); and the hygromycin resistance gene (Santerre, et al., Gene 30:147, 1984).

Alternatively, the nucleic acid construct comprises a detectable marker gene. Suitable detectable marker gene include, for example, a bacterial luciferase gene; a firefly luciferase gene; or a β-galactosidase gene (the expression of which is detected by the metabolism of 5-bromo-4-chloro-3-indolyl-β-D-galactopyranoside to produce a blue precipitate) or a fluorescent marker, such as, for example, a green fluorescent protein (gfp), a monomeric discosoma red fluorescent protein (dsRED) or a monomeric GFP from Aequorea coerulescens.

### Producing an Expression Construct

Methods for producing expression constructs are known in the art and/or described in Ausubel et al (In: Current Protocols in Molecular Biology. Wiley Interscience, ISBN 047 150338, 1987), Sambrook et al (In: Molecular Cloning: Molecular Cloning: A Laboratory Manual, Cold Spring Harbor Laboratories, New York, Third Edition 2001).

Typically, the nucleic acid encoding the constituent components of the expression construct is/are isolated using a known method, such as, for example, amplification (e.g., using PCR or splice overlap extension) or isolated from nucleic acid from an organism using one or more restriction enzymes or isolated from a library of nucleic acids. Methods for such isolation will be apparent to the ordinary skilled artisan. For example, the present inventors have used splice overlap extension to produce nucleic acid encoding an antimicrobial peptide variant or have synthesized nucleic acid encoding an antimicrobial peptide variant.

Alternatively, nucleic acid encoding a nucleic acid constituent of a construct for use in the method of the present invention is isolated using polymerase chain reaction (PCR). Methods of PCR are known in the art and described, for example, in Dieffenbach (ed) and Dveksler (ed) (In: PCR Primer: A Laboratory Manual, Cold Spring Harbour Laboratories, NY, 1995). Generally, for PCR two non-complementary nucleic acid primer molecules comprising at least about 20 nucleotides in length, and more preferably at least 25 nucleotides in length are hybridized to different strands of a nucleic acid template molecule, and specific nucleic acid copies of the template are amplified enzymatically. Preferably the primers hybridize to nucleic acid adjacent to the nucleic acid of interest (e.g., a nucleic acid encoding an antimicrobial peptide and/or analog and/or derivative, a promoter and/or a nucleic acid encoding a detectable marker or selectable marker), thereby facilitating amplification of the nucleic acid. Following amplification, the amplified nucleic acid is isolated using a method known in the art and, preferably cloned into a suitable vector, e.g., a vector described herein.

Other methods for the production of an expression construct of the invention will be apparent to the skilled artisan and are encompassed by the present invention. For example, a nucleic acid encoding a antimicrobial peptide is introduced into a publicly available expression vector in operable connection with a promoter that expresses a peptide in a mammary gland or cell or tissue thereof. Suitable vectors include, for example, a pRβH vector described in U.S. Ser. No. 10/952,376 in which a nucleic acid encoding an antimicrobial peptide analog or derivative is operably linked to a β-casein promoter and a β-lactamase selectable marker gene, or a pVEβcash vector also described in U.S. Ser. No. 10/952,376 in which a nucleic acid encoding an antimicrobial peptide or analog or derivative is operably linked to a truncated β-casein promoter and a neomycin resistance gene.

Prior to introduction of the expression construct into a cell to produce a genetically-modified cell, said construct is preferably linearized, for example, by restriction endonuclease digestion to facilitate integration into the genome of the cell. Optionally, regions that are not required for expression of an antimicrobial peptide and/or analog and/or derivative and/or selectable/detectable marker are removed at this stage, e.g., by restriction endonuclease digestion.

### Production of a Knock-in Construct

In one embodiment, the expression construct is a so-called “knock-in” construct. Such a construct facilitates the insertion of an expression construct at a predetermined site in the genome of a non-human mammal by homologous recombination. Accordingly, such a construct is useful for replacing an endogenous antimicrobial peptide encoding gene with a nucleic acid encoding, for example, an antimicrobial peptide or analog or derivative described according to any embodiment hereof. For example, a nucleic acid encoding an antimicrobial peptide or variant or fusion protein described herein is inserted into the coding region of a gene expressed in milk in nature, e.g., αS1-, αS2-, β-, γ-, and κ-casein, β-lactoglobulin, whey acid protein, or lactalbumin.

One of two configurations of construct is generally used for a vector for homologous recombination, i.e., an insertion construct or a replacement construct. An insertion construct comprises a region of homology to the target nucleic acid cloned as a single continuous sequence. The insertion construct additionally comprises a nucleic acid that is to be inserted into the target nucleic acid, e.g., a antimicrobial peptide encoding nucleic acid, positioned adjacent to and, if required, in-frame with the region of homology. The insertion vector is then linearized, e.g., by cleavage of a unique restriction site within the region of homology with the target sequence. Homologous recombination introduces the insertion construct sequences and any adjacent nucleic acid into the homologous site of the target nucleic acid, interrupting normal target nucleic acid structure by adding an additional sequence. Such a vector is useful for, for example, introducing one or more antimicrobial peptide encoding nucleic acids

A replacement construct is also useful for knocking-in a nucleic acid of interest. This form of construct contains two regions of homology to the target nucleic acid located on either side of a heterologous nucleic acid, for example, encoding one or more antimicrobial peptides or analogs or derivatives and/or selectable/detectable markers. Homologous recombination proceeds by at least two recombination events, i.e., a double cross-over event that leads to the replacement of target nucleic acid with the replacement construct sequences. More specifically, each region of homology in the vector induces at least one recombination event that leads to the heterologous nucleic acid in the vector replacing the nucleic acid located between the regions of homology in the target nucleic acid. A replacement construct is useful for, for example, replacing an endogenous antimicrobial peptide encoding gene with a nucleic acid encoding a specific antimicrobial peptide described herein.

The present invention clearly contemplates an expression construct designed to introduce a nucleic acid encoding a antimicrobial peptide at a predetermined site in the genome of a non-human mammal. In general terms, such an expression construct comprises a nucleic acid comprising a nucleotide sequence that is effective for homologous recombination with the target nucleic acid. For example, a replacement targeting vector comprises at least two regions of nucleic acid that are substantially identical to a genomic sequence of the target sequence. As will be apparent to the skilled artisan, some degree of non-identity does not significantly adversely affect the gene targeting capability of a construct of the invention. However, a higher the degree of identity between the regions of homology in the vector and the target sequence increases the likelihood of effective homologous recombination. Accordingly, it is preferred that a region of a vector homologous to a target nucleic acid comprises a nucleotide sequence that is at least about 80% identical to the target sequence, more preferably 90% or 95% identical.

Longer regions of homology are useful for inducing homologous recombination at the target nucleic acid. However, this region need not be so long so as to be unwieldy. Preferably each region of homology comprises at least about 1500 bp that is substantially identical to a target sequence, more preferably 2000 bp and even more preferably at least about 3000 bp.

Guidelines for the selection and use of sequences are described, for example, in Deng and Cappecchi, Mol. Cell. Biol., 12:3365-3371, 1992 and Bollag, et al., Ann. Rev. Genet., 23:199-225, 1989.

Suitable targeting constructs of the invention are prepared using standard molecular biology techniques known to those of skill in the art. For example, techniques useful for the preparation of suitable vectors are described by Maniatis, et al., Molecular Cloning: A Laboratory Manual, Cold Spring Harbor Laboratory, Cold Spring Harbor, N.Y. An appropriate vector includes, for example, an insertion vector such as the insertion vector described by Capecchi, M. R., Science, 244:1288-92, 1989; or a vector based on a promoter trap strategy or a polyadenylation trap, or “tag-and-exchange” strategy described by Bradley, et al., Biotechnology, 10:543-539, 1992; or Askew, et al., Mol. Cell. Biol., 13:4115-5124, 199.

### Production of a Genetically-Modified Cell

Following production of a suitable expression construct, said expression construct is introduced into a suitable cell. Depending on the type of construct produced the type of cell and method of introduction will vary.

For example, should the expression construct randomly integrate into the genome of a cell and not require homologous recombination, such a construct is preferably introduced into the pronucleus of a fertilized oocyte isolated from an animal to which the construct is to be introduced. Methods for isolating and/or producing a fertilized oocyte will be apparent to the skilled artisan. For example, one or more oocytes are harvested from a female animal and fertilized in vitro. Following a suitable time for the pronucleus to form, the expression construct is injected into said pronucleus. The resulting zygote is then maintained for a time and under conditions sufficient for an embryo to form, e.g., for a blastocyst to form. Optionally, the embryo or a cell thereof is screened to detect presence of the expression construct, e.g., by detecting expression of a selectable or detectable marker gene or by PCR or in situ hybridization. The embryo is then administered to or implanted into a uterus of a non-human mammal and maintained under conditions for a non-human mammal to develop and be born. Such methods for producing a genetically-modified cell and/or animal are known in the art and/or described in U.S. Ser. No. 10/820,777, WO93/14200, WO91/19810, WO93/02189, WO89/00689, WO92/06187, EP0451700, WO92/13069 and WO89/06689. Methods for producing genetically-modified cows using microinjection are described, for example, in Krimpenfort et al., Bio/Technol., 9: 844-847, 1991; Hyttinen et al., Bio/Technol., 12: 606-608, 1994 and/or Eyestone, Theriogeriology, 51: 509-517, 1998.

Alternatively, a genetically-modified non-human mammal is produced by introducing an expression construct or expression vector as described according to any embodiment hereof into a somatic cell, e.g., a fibroblast of a non-human mammal, resulting in a genetically-modified non-human mammalian cell. Methods for introducing a nucleic acid into a somatic cell are known in the art and include, for example, electroporation, microinjection, transfection mediated by DEAE-dextran, transfection mediated by calcium phosphate, transfection mediated by liposomes such as by using Lipofectamine (Invitrogen) and/or cellfectin (Invitrogen), transduction by Adenoviruses, Herpesviruses, Togaviruses or Retroviruses and microparticle bombardment such as by using DNA-coated tungsten or gold particles (Agacetus Inc., WI, USA). The genetically-modified non-human mammalian cell or the nucleus thereof is then injected into or fused with an enucleated mature oocyte from the same species of non-human mammal as the somatic cell to produce a monocell embryo. This monocell embryo is them maintained for a time and under conditions sufficient for a multi-cell embryo to form, e.g., a blastocyst to form, and the multi-cell embryo is then administered to or implanted into a uterus of a non-human mammal and maintained under conditions for a non-human mammal to develop and be born. Exemplary methods for producing a genetically-modified non-human mammal using these methods, e.g., nuclear transfer, will be apparent to the skilled artisan and/or are described, for example, in Schnieke et al. Science, 278: 2131-2133, 1997; and Baguisi et al., Nature Biotech., 17: 456-461, 1999.

In another example, an expression construct is included in a retrovirus particle, preferably containing the envelope protein from vesicular somatic protein. The retrovirus particle is injected between the zona pellucida and the membrane of an oocyte of a non-human mammal at a period when the nuclear membrane is absent, e.g., during metaphase II (MII) of the second meiosis. The infected oocyte is then fertilized with a sperm cell, and incubated for a time and under conditions sufficient for an embryo, e.g., a blastocyst to form. The embryo is then administered to, e.g., implanted in a uterus of a female non-human mammal and a genetically-modified non-human mammal permitted to develop and be born. A suitable retrovirus-mediated method for producing a genetically-modified non-human mammal, e.g., a cow or bull, is described in Chan et al., Proc Natl Acad Sci USA. 95: 14028-33, 1998.

It is to be understood that the genetically modified non-human mammals described herein can be produced by methods other than the specific methods taught herein, e.g., sperm-mediated transgenesis or embryonic stem cell-mediated transgenesis

### Production of a Genetically-Modified Non-Human Mammal

A fertilized oocyte or a zygote or an embryo into which an expression construct of the invention has been introduced is preferably transferred to a uterus of a pregnant or pseudopregant female non-human mammal and allowed to develop into an entire mammal. Following birth, animals are screened to identify those carrying the expression construct using a method known in the art and/or described herein.

Generally, such a method results in production of a non-human mammal heterozygous for the expression construct of the invention or in which some cells comprise the expression construct and some do not, i.e., a chimeric non-human mammal. By breeding either the non-human mammal heterozyogous for the expression construct or the chimeric non-human mammal with a wild-type non-human mammal offspring that are heterozygous for the expression construct are produced. Breeding two non-human mammals heterozygous for the expression construct or two non-human mammals homozygous for the expression construct or a non-human mammal heterozygous for the expression construct and a non-human mammal homozygous for the expression construct produces at least some offspring that are homozygous for the expression construct.

The present invention clearly contemplates either a genetically-modified non-human mammal homozygous for a genetic construct as described according to any embodiment hereof or a genetically-modified non-human mammal heterozygous for a genetic construct as described according to any embodiment hereof.

The present invention additionally contemplates a cell, a cell line, a cell culture, a primary tissue, a cellular extract or a cell organelle isolated from a genetically-modified non-human mammal of the present invention. For example, a cell culture or cell line or cell is derived from any desired tissue or cell-type from the genetically-modified non-human mammal, e.g., a mammary gland or cell or tissue thereof, or a stem cell or a reproductive cell, e.g., an oocyte or a sperm.

Isolation of an Antimicrobial Peptide and/or Analog and/or Derivative from Milk

Suitable methods for isolating an antimicrobial peptide and/or analog and/or derivative have been described supra and are to be taken to apply mutatis mutandis to isolation from milk.

In on example, an antimicrobial peptide and/or analog and/or derivative as described according to any embodiment hereof is isolated from milk by chromatography and concentration. Different types of chromatography can be employed and include ion exchange chromatography, reverse phase chromatography, molecular exclusion chromatography or affinity chromatography. The ion exchange chromatography can be anion exchange chromatography. The affinity chromatography can be immunoaffinity chromatography. Furthermore, multiple chromatography steps may be performed.

For example, an antimicrobial peptide and/or analog and/or derivative is isolated form milk by clarifying the milk of a non-human genetically-modified mammal as described according to any embodiment hereof, resulting in a clarified milk, and subjecting the clarified milk to chromatography, thereby isolating the peptide and/or analog and/or derivative.

In another example, an antimicrobial peptide and/or analog and/or derivative is isolated from milk by clarifying the milk of a non-human genetically-modified mammal as described according to any embodiment hereof, resulting in a clarified milk, subjecting the clarified milk to expanded-bed anion exchange chromatography, resulting in an anion exchange chromatographed material, subjecting the anion exchange chromatographed material to reverse phase chromatography, resulting in a reverse phase chromatographed material, subjecting the reverse phase chromatographed material to anion exchange chromatography, resulting in an anion exchange chromatographed material, subjecting the anionic exchange chromatographed material to molecular exclusion chromatography, resulting in a molecular exclusion chromatographed material, concentrating the molecular exclusion chromatographed material, resulting in a concentrated material, and subjecting the concentrated material to molecular exclusion chromatography, thereby isolating the antimicrobial peptide and/or analog and/or derivative.

### Determining the Antimicrobial Activity of a Peptide

Methods for determining the antimicrobial activity of a peptide will be apparent to the skilled artisan, for example, based on the description herein. For example, as exemplified herein, the present inventors have used a radial diffusion assay.

Other suitable methods include, for example, a broth dilution method. Essentially, this method involves growing a microorganism in liquid media until log phase is reached. The peptide, analog or derivative to be tested is serially diluted in media in which the microorganism is grown are grown and a sample of the microorganism added to the peptide containing sample. The sample is then maintained for a time and under conditions sufficient for growth of the microorganism, and the amount of growth of the microorganism determined relative to a negative control by detecting the absorbance at A₆₀₀.

Another method in accordance with the invention comprises contacting a microorganism previously contacted with a peptide to be tested with an agent that has affinity for a compound located within the microorganism, but is not able to cross an intact or undamaged membrane. The presence of the agent within the microorganism indicates that the agent crossed the membrane indicating that the membrane of the microorganism was damaged by the peptide. An example of such an agent is Sytox green dye (Molecular Probes, Eugene, Oreg.). This dye has a strong affinity for nucleic acids, but can only penetrate cells that have a damaged membrane.

Yet another method for determining whether a peptide being assayed for antimicrobial activity has damaged the membrane of the microorganism involves contacting the microorganism with a test peptide and an agent capable of crossing the membrane of the microorganism. The agent is capable of being processed within the microorganism to form a product that is unable to cross an undamaged membrane. The medium surrounding the microorganism is then assayed for the presence of said product. The presence of said product in the medium in which the microorganism is grown is indicative of damage to the membrane of the microorganism caused by the peptide, and is indicative of the antimicrobial activity of the peptide. An example of a suitable agent is calcein AM. Calcein AM is converted into free calcein within the microorganism. Normally, free calcein is unable to cross the cell membrane of the microorganism and enter the surrounding culture. Thus, detection of free calcein in the medium surrounding the microorganism is indicative of damage to the cell membrane of the microorganism, and thus the antimicrobial activity of the peptide.

### Identifying Peptides/Analogs/Derivatives for Treating Mastitis

Methods for testing a genetically-modified non-human mammal as described according to any embodiment hereof for protection against mastitis will be apparent to the skilled artisan.

In one example, a genetic construct is tested for its ability to protect against mastitis prior to use to produce a genetically-modified non-human mammal. For example, an in vitro assay is performed essentially as described in Almeida et al., J. Vet. Pharmacol. Ther., 30: 151-156, 2007, however, modified to test a genetic construct as described according to any embodiment hereof. For example, a mammary epithelial cell line, e.g., a bovine mammary epithelial cell line such as MAC-T is transfected with an expression construct or expression vector as described according to any embodiment hereof and/or contacted with a peptide and/or analog and/or derivative as described according to any embodiment hereof. Following sufficient time for a peptide or analog or derivative to be expressed and/or secreted the transfected cell is contacted with a bacterium that causes mastitis, e.g., S. uberis (e.g., a strain designated UT888) or S. dysgalactiae subsp. dysgalactiae (e.g., a strain designated UT19) or S. aureus (e.g., a strain designated UT23). The bacteria are fluorescently labelled, e.g., with a LIVE/DEAD BacLight viability kit from Molecular Probes, Eugene, Oreg., USA. Following washing to remove unbound or non-internalized bacteria, cells are viewed under a fluorescent microscope to determine the number of cells internalized and, of those cells internalized the number of cells that are viable. A peptide and/or analog and/or derivative or expression construct or expression vector that reduces the number of internalized and/or viable internalized bacterial cells is considered useful for preventing mastitis.

In another example, a mammary epithelial cell is contacted with bacteria prior to treatment, e.g., transfection with an expression construct or expression vector as described according to any embodiment hereof and/or contact with a peptide and/or analog and/or derivative as described according to any embodiment hereof. The level of internalized and/or viable internalized bacterial cells is then determined and a peptide and/or analog and/or derivative or expression construct or expression vector that reduces the number of internalized and/or viable internalized bacterial cells is considered useful for preventing mastitis.

In a further example, a genetically-modified non-human mammal is produced by performing a method as described according to any embodiment hereof. At an appropriate time, e.g., during pregnancy and/or during lactation bacteria (e.g., S. uberis) that cause mastitis are infused through a teat canal into a teat cistern using, for example, a syringe. Following a sufficient time for an infection to occur and mastitis to develop the non-human mammal is assessed for mastitis development and infection, e.g., by determining the level of somatic cells in milk, and/or culturing bacteria growing in milk. Examples of suitable methods for infusing bacteria into a mammary gland and/or assessing infection/mastitis status will be apparent to the skilled artisan and/or described, for example, in Nickerson et al., J. Dairy Sci., 73: 2774-2784, 1990.

In another example, a non-human mammal is infected with bacteria that cause mastitis and then a peptide and/or analog and/or derivative and/or expression construct is administered to the mammal. Following sufficient time, e.g., for mastitis to develop and/or for a peptide and/or analog and/or derivative to kill or prevent growth of the bacteria and/or for a peptide to be expressed from said expression construct and to kill or prevent growth of the bacteria, the level of infection and/or mastitis is assessed e.g., as described supra.

In another example, milk is isolated from a genetically-modified non-human mammal as described according to any embodiment hereof, and the milk is contacted with a sample of bacteria that cause mastitis, e.g., in a radial diffusion method and/or a broth dilution assay as described herein. An exemplary method is described in Wall et al., Nature Biotech., 23: 445-451, 2005.

### Compositions Comprising an Antimicrobial Peptide, Analog or Derivative

For a composition to be administered to a mammary gland or a cell or tissue thereof, a preferred composition comprises a carrier or excipient is suitable for intra-mammary administration. For example, the carrier or excipient does not inhibit the activity of the peptide and/or analog and/or derivative even following administration to a mammary gland. Preferably the carrier or excipient does not cause inflammation in a mammary gland or a tissue thereof. Preferably the carrier does not change the constitution of milk produced from the mammary gland and/or a milk product produced there from. In one example, the carrier or excipient is a non-phosphate containing isotonic buffer at the physiological pH of milk i.e. pH 6.7.

In another example, the carrier or excipient is a liquid, for example, a physiologic salt solution containing non-phosphate buffer at pH 6.7 to 7.6.

For intra-mammary administration by injection into a mammary gland, a preferred carrier or excipient is oil-based, preferably using mineral oil.

An antimicrobial peptide and/or analog and/or derivative as described according to any embodiment hereof and/or produced by a method as described according to any embodiment hereof is preferably provided in a composition, e.g., a pharmaceutical composition, a disinfecting composition, a preservative composition, a cosmetic composition or a phytoprotective composition. Such a composition additionally comprises, for example, a suitable carrier, e.g., pharmaceutically acceptable carrier. The term “carrier” as used herein, refers to a carrier that is conventionally used in the art to facilitate the storage, administration, and/or the biological activity of a regulatory agent. A carrier may also reduce any undesirable side effects of the regulatory agent. A suitable carrier is stable, i.e., incapable of reacting with other ingredients in the formulation. The carrier does not produce significant local or systemic adverse effect in recipients at the dosages and concentrations employed for treatment. Such carriers are generally known in the art. Suitable carriers for this invention include those conventionally used. Water, saline, aqueous dextrose, and glycols are preferred liquid carriers, particularly (when isotonic) for solutions. Alternatively, the carrier is selected from various oils, including those of petroleum, animal, vegetable or synthetic origin, for example, peanut oil, soybean oil, mineral oil, sesame oil, and the like. Suitable pharmaceutical carriers include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like.

A composition comprising an antimicrobial peptide of the invention or a derivative or analog thereof can be subjected to conventional pharmaceutical expedients, such as sterilization, and can contain conventional pharmaceutical additives, such as preservatives, stabilizing agents, wetting, or emulsifying agents, salts for adjusting osmotic pressure, buffers, and the like. Other acceptable components in the composition of the invention include, but are not limited to, isotonicity-modifying agents such as water, saline, and buffers including phosphate, citrate, succinate, acetic acid, and other organic acids or their salts. However, because an antimicrobial peptide of the present invention is a soluble hydrophilic molecule, sprays, solutions, lotions and topical ointments for administration are readily formulated without the need for chemical solvent-based solubilizing agents, which may be detrimental to a subject to which the peptide is to be administered.

Preferably a composition of the invention also includes one or more stabilizers, reducing agents, anti-oxidants and/or anti-oxidant chelating agents. The use of buffers, stabilizers, reducing agents, anti-oxidants and chelating agents in the preparation of protein-based compositions, is known in the art and described, for example, in Wang et al. J. Parent. Drug Assn. 34:452-462, 1980; Wang et al. J. Parent. Sci. Tech. 42:S4-S26 (Supplement), 1988. Suitable buffers include acetate, adipate, benzoate, citrate, lactate, maleate, phosphate, tartarate, borate, tri(hydroxymethyl aminomethane), succinate, glycine, histidine, the salts of various amino acids, or the like, or combinations thereof. Suitable salts and isotonicifiers include sodium chloride, dextrose, mannitol, sucrose, trehalose, or the like. Where the carrier is a liquid, it is preferred that the carrier is hypotonic or isotonic with oral, conjunctival, or dermal fluids and has a pH within the range of 4.5-8.5. Where the carrier is in powdered form, it is preferred that the carrier is also within an acceptable non-toxic pH range.

In some embodiments, an antimicrobial peptide of the invention or analog or derivative thereof is incorporated within a composition for administration to a mucus membrane, e.g., by nasal administration. Such a composition generally includes a biocompatible polymer functioning as a carrier or base. Such polymer carriers include polymeric powders, matrices or microparticulate delivery vehicles, among other polymer forms. The polymer can be of plant, animal, or synthetic origin. Often the polymer is crosslinked. Additionally, in these delivery systems the biologically active agent, can be functionalized in a manner where it can be covalently bound to the polymer and rendered inseparable from the polymer by simple washing. Polymers useful in this respect are desirably water interactive and/or hydrophilic in nature to absorb significant quantities of water, and they often form hydrogels when placed in contact with water or aqueous media for a period of time sufficient to reach equilibrium with water.

Drug delivery systems based on biodegradable polymers are preferred in many biomedical applications because such systems are broken down either by hydrolysis or by enzymatic reaction into non-toxic molecules. The rate of degradation is controlled by manipulating the composition of the biodegradable polymer matrix. These types of systems can therefore be employed in certain settings for long-term release of biologically active agents. Examples of suitable biodegradable polymers include, for example, poly(glycolic acid) (PGA), poly-(lactic acid) (PLA), and poly(D,L-lactic-co-glycolic acid) (PLGA).

Alternatively, a peptide or analog or derivative thereof of the invention can be administered via in vivo expression of the recombinant protein. In vivo expression can be accomplished via somatic cell expression according to suitable methods (see, e.g. U.S. Pat. No. 5,399,346). In this embodiment, nucleic acid encoding the protein can be incorporated into a retroviral, adenoviral or other suitable vector (preferably a replication deficient infectious vector) for delivery, or can be introduced into a transfected or transformed host cell capable of expressing the protein for delivery. In the latter embodiment, the cells can be implanted (alone or in a barrier device), injected or otherwise introduced in an amount effective to express the protein in a therapeutically effective amount.

In another embodiment, the antimicrobial peptides of the invention are used in combination with or to enhance the activity of other antimicrobial agents or antibiotics. Combinations of the peptides with other agents may be useful to allow antibiotics to be used at lower doses due to toxicity concerns, to enhance the activity of antibiotics whose efficacy has been reduced or to effectuate a synergism between the components such that the combination is more effective than the sum of the efficacy of either component independently. Antibiotics that may be combined with an antimicrobial peptide in combination therapy include but are not limited to penicillin, ampicillin, amoxycillin, vancomycin, cycloserine, bacitracin, cephalolsporin, methicillin, streptomycin, kanamycin, tobramycin, gentamicin, tetracycline, chlortetracycline, doxycycline, chloramphenicol, lincomycin, clindamycin, erythromycin, oleandomycin, polymyxin nalidixic acid, rifamycin, rifampicin, gantrisin, trimethoprim, isoniazid, paraminosalicylic acid, and ethambutol.

As exemplified herein, a peptide and/or analog and/or derivative of the present invention has a synergistic effect on the antimicrobial activity of chloramphenicol and/or tetracycline. Accordingly, in a preferred example, the present invention provides a composition comprising a peptide and/or analog and/or derivative of the present invention and tetracycline or a structurally and functionally-related antibiotic and/or chloramphenicol and/or a structurally and functionally-related antibiotic.

In another embodiment, the composition is a disinfecting or preservative composition, e.g., for cleaning a surface and/or for preserving food or pharmaceuticals. Such a composition comprises a suitable carrier, such as, for example, as described supra. Such a composition also preferably comprises one or more protease inhibitors to reduce or prevent degradation of the antimicrobial peptide of the invention.

In another embodiment, the composition is a phytoprotective composition. Such a composition is, for example, sprayed onto or applied to a plant or soil in which a plant is grown or is to be grown to prevent a microbial infection or to treat a microbial infection.

As will be apparent to the skilled artisan based on the foregoing, a preferred composition is suitable for spray application. For example, the composition is suitable for spraying onto a food product or onto a food preparation surface or onto a plant. Such spray compositions are useful for the treatment of food, e.g., to prevent food spoilage without actually handling the food. The skilled artisan will be aware of suitable components of a composition suitable for spray application. For example the composition comprises an antimicrobial peptide or analog or derivative as described according to any embodiment hereof and a suitable carrier, e.g., water or saline. Such a composition may also comprise, for example, a surfactant, e.g., Tween 20, preferably a surfactant does not inhibit or reduce the antimicrobial activity of said peptide, analog or derivative.

In some embodiments, a peptide described according to any embodiment hereof is applied to a surface of a device to prevent microbial proliferation on that surface of the device. The device is, for example, a medical device, which includes any material or device that is used on, in, or through a patient's body in the course of medical treatment (e.g., for a disease or injury). Medical devices include but are not limited to such items as medical implants, wound care devices, drug delivery devices, and body cavity and personal protection devices. The medical implants include but are not limited to urinary catheters, intravascular catheters, dialysis shunts, wound drain tubes, skin sutures, vascular grafts, implantable meshes, intraocular devices, heart valves, prosthetic devices (e.g., hip prosthetics) and the like. Wound care devices include but are not limited to general wound dressings, biologic graft materials, tape closures and dressings, and surgical incise drapes. Drug delivery devices include but are not limited to needles, drug delivery skin patches, drug delivery mucosal patches and medical sponges.

### Routes of Administration

In one example, a peptide and/or analog and/or derivative or composition comprising same is administered to a mammary gland to thereby treat or prevent mastitis. In accordance with this embodiment, the peptide and/or analog and/or derivative or composition is infused into a canal of a mammary gland, e.g., by dipping a teat or mammary gland into a composition peptide and/or analog and/or derivative or by infusion by injection into a canal of a mammary gland. Alternatively, the peptide and/or analog and/or derivative or composition is injected directly into a mammary gland or a region thereof.

In one example, an expression construct or expression vector of the present invention is administered to a mammary gland or a cell or tissue thereof by high-pressure jet-injection, e.g., as described in Kerr et al., Anim. Biotechnol., 7: 33-45, 1996 or Zheng et al., Gene, 353: 107-117, 2005. For example, using high-pressure jet-injection Kerr et al., delivered naked DNA into parenchyma of lactating sheep parenchyma.

In another example, an expression construct or expression vector of the present invention is administered to a mammary gland or cell or tissue thereof in a virus, e.g., an adenovirus. For example, a replication defective adenovirus, e.g., human adenovirus comprising an expression construct of the invention is produced and infused or injected into a canal of a mammary gland. For example, U.S. Pat. No. 6,875,903 describes suitable methods for administering an adenovirus comprising an expression construct to a mammary gland or a cell or tissue thereof of a ruminant animal.

Other viruses will be apparent to the skilled artisan and include, for example, a retrovirus, e.g., a lentivirus, or an adeno-associated virus.

For example, a retroviral vector generally comprises cis-acting long terminal repeats (LTRs) with packaging capacity for up to 6-10 kb of foreign sequence. The minimum cis-acting LTRs are sufficient for replication and packaging of a vector, which is then used to integrate the expression construct into the target cell to provide expression. Widely used retroviral vectors include those based upon murine leukemia virus (MuLV), gibbon ape leukemia virus (GaLV), simian immunodeficiency virus (SW), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:274-2378 (1989); Miller et al., J. Virol. 65:2220-2224 (1991); PCT/US94/05700; Miller and Rosman BioTechniques 7:980-990, 1989; Miller, A. D. Human Gene Therapy 1:5-14, 1990; Scarpa et al) Virology 180:849-852, 1991; Burns et al. Proc. Natl. Acad. Sci. USA 90:8033-8037, 1993.).

Various adeno-associated virus (AAV) vector systems have also been developed for nucleic acid delivery. AAV is a helper-dependent DNA parvovirus which belongs to the genus Dependovirus. AAV has no known pathologies and is incapable of replication without additional helper functions provided by another virus, such as an adenovirus, vaccinia or a herpes virus, for efficient replication and a productive life cycle. In the absence of the helper virus, AAV establishes a latent state by insertion of its genome into a host cell chromosome. Subsequent infection by a helper virus rescues the integrated copy which can then replicate to produce infectious viral progeny. The combination of the wild type AAV virus and the helper functions from either adenovirus or herpes virus generates a recombinant AVV (rAVV) that is capable of replication. One advantage of this system is its relative safety (For a review, see Xiao et al., (1997) Exp. Neurol. 144: 113-124). Vectors containing as little as 300 base pairs of AAV can be packaged and can integrate. Space for exogenous DNA is about 4.7 kb, which is sufficient to incorporate a nucleic acid encoding a peptide or analog or derivative of the present invention. An AAV vector such as that described in Tratschin et al., (1985) Mol. Cell. Biol. 5: 3251-3260 can be used to introduce DNA into cells. A variety of nucleic acids have been introduced into different cell types using AAV vectors (see for example Hermonat et al., (1984) PNAS USA 81: 6466-6470; Tratschin et al., (1985) Mol. Cell. Biol. 4: 2072-2081; Wondisford et al., (1988) Mol. Endocrinol. 2: 32-39; Tratschin et al., (1984) J. Virol. 51: 611-619; and Flotte et al., (1993) J. Biol. Chem. 268: 3781-3790). An AAV-based expression vector typically includes the 145 nucleotide AAV inverted terminal repeats (ITRs) flanking a restriction site that can be used for subcloning of a desired nucleic acid, either directly using the restriction site available, or by excision of the desired nucleotide sequence with restriction enzymes followed by blunting of the ends, ligation of appropriate DNA linkers, restriction digestion, and ligation into the site between the ITRs. For additional detailed guidance on AAV technology which may be useful in the practice of the subject invention, including methods and materials for the incorporation of a nucleotide sequence, the propagation and purification of the recombinant AAV vector containing the nucleotide sequence, and its use in transfecting cells and mammals, see e.g. Carter et al, U.S. Pat. No. 4,797,368 (10 Jan. 1989); Muzyczka et al, U.S. Pat. No. 5,139,941 (18 Aug. 1992); Lebkowski et al, U.S. Pat. No. 5,173,414 (22 Dec. 1992); Srivastava, U.S. Pat. No. 5,252,479 (12 Oct. 1993); Lebkowski et al, U.S. Pat. No. 5,354,678 (11 Oct. 1994); Shenk et al, U.S. Pat. No. 5,436,146 (25 Jul. 1995); Chatterjee et al, U.S. Pat. No. 5,454,935 (12 Dec. 1995), Carter et al WO 93/24641 (published 9 Dec. 1993), and Natsoulis, U.S. Pat. No. 5,622,856 (Apr. 22, 1997).

Additional Uses of an Antimicrobial Peptide and/or Analog and/or Derivative

The antimicrobial activity of the peptides of the present invention also make them useful for treating or preventing an infection in a subject. Accordingly, the present invention also provides a method of therapeutic or prophylactic treatment of a subject comprising administering the peptide, analog, derivative, fusion protein, complex, expression construct or expression vector or composition of the invention or composition comprising same to a subject in need thereof. In this respect, a subject in need of treatment with a peptide, analog or derivative of the invention is, for example, a subject suffering from an infection or suspected of suffering from an infection or at risk of developing an infection.

As used herein, the term “subject” shall be taken to mean any animal, including a human, plant or insect that may be infected by a microorganism. Preferably the subject is any animal, including a human that may be infected by a microorganism against which a peptide, analog, derivative, fusion protein, complex or composition of the invention has antimicrobial activity.

Preferably the peptide is administered under conditions sufficient for the peptide, analog and/or derivative to reduce or prevent microbial growth and/or to kill a microorganism, e.g., in a pharmaceutical composition or a cosmetic composition.

As used herein, the term “infection” shall be taken to mean the invasion, development and/or multiplication of a microorganism within or on another organism. An infection may be localized to a specific region of an organism or systemic. Infections for which a peptide, analog and/or derivative of the invention are useful for treating include any infection caused by a bacteria or a fungus and will be apparent to the skilled artisan from the disclosure herein.

In this respect, the present invention is not limited to the treatment of an infection in an animal subject. Rather, a peptide, analog, derivative, fusion protein, complex or composition of the present invention is also useful for, for example, treatment of a plant to thereby reduce or prevent a microbial infection therein or thereon. Accordingly, the peptide of the invention or analog or derivative thereof is a phytoprotective agent.

In a preferred embodiment, the subject is an animal, and more preferably a mammal. Accordingly, the peptide of the invention or analog or derivative thereof is a pharmaceutical agent or a cosmetic agent.

The peptide, analog and/or derivative of the invention can be administered to a subject by any of a variety of means, such as, for example, topical administration, nasal administration, oral administration, vaginal administration, rectal administration, intravenous administration, intraperitoneal administration, or subcutaneous administration. For example, as infectious microorganisms generally enter a mammal by way of a membrane, e.g., a mucus membrane, a peptide, analog or derivative of the invention is preferably administered in a manner suitable to contact a membrane.

In a preferred embodiment, a method of treating a subject of the invention additionally comprises providing or obtaining the peptide, analog, derivative, fusion protein, complex or composition of the invention or information concerning same.

As will be apparent to the skilled artisan based on the foregoing, the present invention also provides for the use of a peptide, analog, derivative, fusion protein, complex, expression construct or expression vector or composition of the invention in medicine. For example, the present invention provides for the use of the peptide, analog, derivative, fusion protein, complex, expression construct or expression vector of the present invention in the manufacture of a medicament for the treatment or prophylaxis of an infection.

The present inventors have demonstrated that the peptides of the present invention are active against a variety of microorganisms. Accordingly, the present invention also provides a method for reducing or preventing microbial growth, said method comprising contacting a microorganism or a surface or composition of matter suspected of comprising a microorganism with a peptide, analog, derivative, fusion protein, complex or composition of the invention for a time and under conditions sufficient to reduce microbial growth and/or kill a microorganism, thereby reducing or preventing microbial growth. Such a method is suitable for, for example, disinfecting a surface and/or preserving a food product and/or reducing or preventing water contamination.

Alternatively, or in addition, the method comprises applying a peptide, analog, derivative, fusion protein, complex or composition of the invention to a surface or composition of matter suspected of comprising a microorganism for a time and under conditions sufficient to reduce microbial growth and/or kill a microorganism, thereby reducing or preventing microbial growth. For example, the peptide, analog, derivative, fusion protein, complex or composition of the invention is sprayed onto the surface or composition of matter. Such spray application is useful for, for example, applying a peptide, analog or derivative of the invention to a food product or a fluid to be consumed, e.g., by a human. This is because spraying the peptide, analog, derivative, fusion protein, complex or composition reduces the handling of said food product or fluid, thereby further reducing the risk of microorganism contamination.

In one embodiment, the method additionally comprises performing a method to detect the presence of a microorganism. Such a detection method may be performed prior to and/or following contacting with a peptide, analog, derivative, fusion protein, complex or composition of the invention.

As will be apparent to the skilled artisan based on the foregoing, the present invention also provides for the use of a peptide, analog, derivative, fusion protein or complex of the invention in the manufacture of a composition for reducing or preventing microbial growth.

As a peptide of the present invention is useful for reducing microbial growth in a food product, the present invention additionally provides a method for prolonging the storage life of a perishable product, said method comprising:
(i) contacting a perishable product with the peptide, analog, derivative, fusion protein, complex or composition of the present invention for a time and under conditions sufficient to reduce or prevent growth of a microorganism and/or to kill a microorganism; and
(ii) storing the perishable product.

In this respect, the perishable product is capable of being stored for a longer period of time than the same product that has not been contacted with the peptide, analog, derivative, fusion protein, complex or composition of the invention.

Moreover, the present invention also provides a method for preserving milk or a milk product, said method comprising obtaining milk from a genetically-modified non-human mammal as described according to any embodiment hereof or a milk product produced there from, wherein said milk or milk product comprises an antimicrobial peptide and/or analog and/or derivative of the invention that is active therein, thereby preserving the milk or milk product.

In one example, the method comprises obtaining milk from a genetically-modified non-human mammal as described according to any embodiment hereof and producing a milk product there from.

The present invention also provides milk or a milk product comprising a peptide and/or analog and/or derivative of the present invention.

The present invention is described further in the following non-limiting examples.

### Example 1

### Peptides Having Antimicrobial Activity Against a Variety of Microorganisms Including Mastitis Causing Microorganisms

This example demonstrates the antimicrobial profile of synthetic peptides designated AGG01 (SEQ ID NO: 7) and AGG02 (SEQ ID NO: 8) against agents of mastitis.

### Synthetic Peptides

Two amidated peptides were commercially synthesized by Auspep. The sequences of the peptides are as follows:

(SEQ ID NO: 7)
KRGFGKKLRKRLKKFRNSIKKRLKNFNVVIPIPLP-NH₂;
and
(SEQ ID NO: 8)
KRGLWESLKRKATKLGDDIRNTLRNFKIKFPVPRQ-NH₂.

### Antimicrobial Assays

Peptides were tested for antimicrobial activity against Streptococcus uberis, Escherichia coli DH5α, Escherichia coli DH5α comprising an ampicillin resistant gene, Pseudomonas spp., Pseudomonas vulgaris, Proteus vulgaris, Pseudomonas aeruginosa (ATCC 27853), Salmonella choleraesuis (ATCC 14028), Bacillus subtilis, Staphylococcus aureus (ATCC 25923), Streptococcus pyogenes (ATCC 19615), Streptococcus Agalactiae (ATCC 12927), Streptococcus equi equi (β-Haemolytic streptococcus) (ATCC 9527), and the yeast Candida albicans (ATCC753), by a two stage radial diffusion assay essentially as described in Steinberg and Lehrer, Methods Mol. Biol., 78: 169-88, 1997. Briefly, approximately 4×10⁶ of mid-logarithmic-phase organisms were grown on plates in 11 ml of warm 0.8% agarose containing 0.03% (w/v) Trytpicase soy broth (TSB) powder, with or without 100 mM NaCl, buffered with 10 mM sodium phosphate, pH 7.4. In the case of S. uberis bacteria were grown on plates comprising 5% horse serum. The test peptide was serially diluted in acidified water (0.01% acetic acid), and 5 μl of diluted peptide sample was loaded in a 2.5 diameter well in the agarose. A 10 ml overlay gel composed of 6% TSB, 0.8% agarose and 10 mM sodium phosphate buffer (pH 7.4) was poured into each well. Plates were then incubated overnight to allow the surviving organisms to form microcolonies. The clear zone were measured to the nearest 0.1 mm using a magnified transilluminator and expressed in units (1 mm=10 U) after subtracting the well diameter. The minimum inhibitory concentration (MIC) is defined by the χ intercept of a regression line through zone diameters obtained from a series of serially diluted peptide samples.

### Results

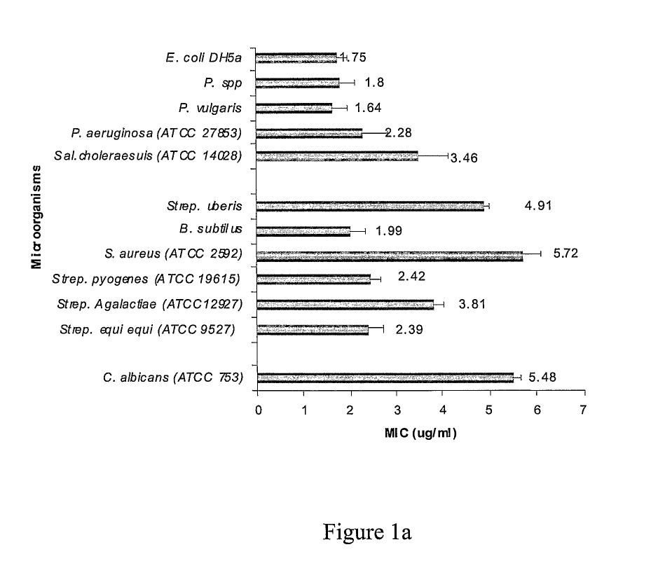

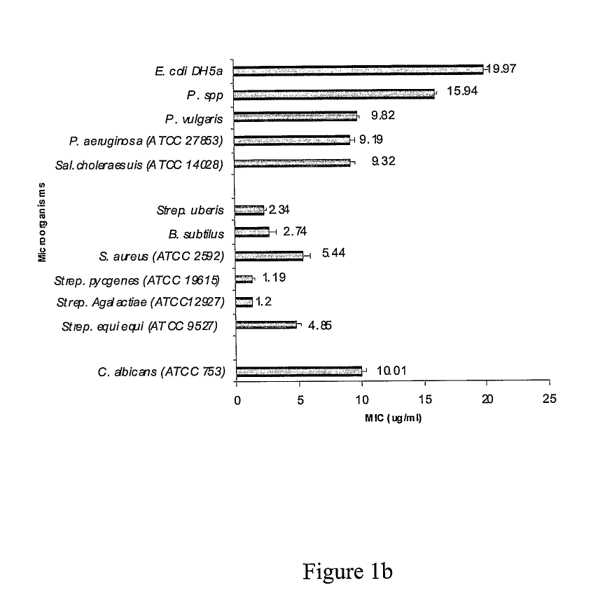

Table 1 shows the minimum inhibitory concentration (MIC) of each of the antimicrobial peptides set forth in SEQ ID Nos: 7 and 8 required to inhibit a range of gram-negative bacteria, gram positive bacteria and a fungus. Data in Table 1 are presented as means±standard error of the mean (SEM) from two experiments. Partial inhibition without obvious definition of a clear zone is indicated by asterisks (**). The MICs obtained for a peptide comprising an amino acid sequence set forth in SEQ ID Nos: 7 and 8 in low salt are also represented graphically in FIGS. 1a and 1b.

TABLE 1

| | MIC (μg/ml) | | | |
|---|---|---|---|---|
| | in media containing 0 mM NaCl or 100 mM NaCl | | | |
| | SEQ ID NO: 7 | | SEQ ID NO: 8 | |
| Microorganism | 0 mM | 100 mM | 0 mM | 100 mM |

Gram-negative bacteria

| | | | | |
|---|---|---|---|---|
| S. uberis | 4.91 ± 0.11 | 6.60 ± 0.18 | 2.34 ± 0.18 | 17.67 ± 0.13 |
| E. coli DH5α | 1.75 ± 0.22 | 1.32 ± 0.35 | 19.97 ± 0.41 | 26.10 ± 0.41 |
| Pseudomonas spp | 1.80 ± 0.30 | 1.83 ± 0.23 | 15.94 ± 0.29 | 22.12 ± 0.43 |
| P. aeruginosa | 2.28 ± 0.53 | 1.51 ± 0.51 | 9.19 ± 0.41 | 10.45 ± 0.24 |
| (ATCC 28753) | | | | |
| Salmonella | 3.46 ± 0.66 | 2.05 ± 0.62 | 9.32 ± 0.38 | ** |
| choleraesuis | | | | |
| (ATCC 14028) | | | | |
| Proteus vulgaris | 1.64 ± 0.32 | 1.73 ± 0.23 | 9.82 ± 0.28 | 67.45 ± 0.20 |

Gram-positive bacteria

| | | | | |
|---|---|---|---|---|
| Bacillus subtilis | 1.99 ± 0.38 | 13.83 ± 0.45 | 2.74 ± 0.48 | 8.67 ± 0.39 |
| Staphylococcus aureus | 5.72 ± 0.37 | ** | 5.44 ± 0.49 | ** |
| (ATCC 25923) | | | | |
| Streptococcus | 2.42 ± 0.25 | 3.57 ± 0.41 | 1.19 ± 0.37 | 8.24 ± 0.37 |
| pyogenes | | | | |
| (ATCC 19615) | | | | |
| Streptococcus | 2.39 ± 0.35 | 4.05 ± 0.39 | 4.85 ± 0.35 | ** |
| equi equi | | | | |
| (ATCC 9527) | | | | |
| Streptococcus agalactiae | 3.81 | | 1.2 | |
| (ATCC 12927) | | | | |

Fungus

| | | | |
|---|---|---|---|
| Candida albicans | 5.48 ± 0.16 | ** | 10.01 ± 0.47 |
| (ATCC 753) | | | |

The data presented in Table 1 and FIGS. 1a and 1b indicate a broad spectrum of activity for the identified antimicrobial peptides, in low and high salt concentrations. The maintenance of antimicrobial activity in high salt suggests efficacy in body fluids, such as, for example, blood.

SEQ ID NO: 7 appears active against all microorganisms tested at less than 10 μg/ml, these low MIC values suggesting that the base peptide and analogs and derivatives thereof having enhanced activity and/or half-life, are particularly strong candidates for development into therapeutic formulations. SEQ ID NO: 7 was capable of inhibiting growth of and/or killing S. uberis in both high and low salt concentrations. Moreover, SEQ ID NO: 7 also exhibited antimicrobial activity against other pathogens that cause mastitis, e.g., E. coli, S. aureus and S. agalactiae.

SEQ ID NO: 8 also has antimicrobial activity against S. uberis and other organisms causative of mastitis e.g., E. coli, S. aureus and S. agalactiae.

In separate experiments, the peptide AGG01 (SEQ ID NO: 7) or AG002 (SEQ ID NO: 8) is shown to be suitable for use in a dairy starter culture comprising lactobacilli, especially one or more Lactis spp., in particular one or more organisms selected individually or collectively from the group consisting of: L. helveticus, L. acidophilus, L. lactis, L. bugaricus and L. citrovorum, and especially L. acidophilus. This low activity against lactobacilli suggests utility of the peptides in dairy starter cultures.

### Example 2

### Production Of Additional Antimicrobial Peptides by Mutagenesis

This example demonstrates the production of new synthetic antimicrobial peptides by evolution of antimicrobial peptides of the invention and C-termini of cathelicidin proteins.

### Peptide Synthesis

Several mutagenesis approaches were employed to generate peptides having antimicrobial activity based on the sequences of peptides comprising SEQ ID NO: 7 and/or 8.

In a first process, the nucleotide sequences of nucleic acids encoding SEQ ID Nos: 7 and 8 were aligned, and codons encoding variable amino acids identified. A nucleotide sequence was then determined that was capable of encoding a sequence comprising an amino acid at any position that occurs in either SEQ ID NO: 7 or SEQ ID NO: 8. This consensus nucleotide sequence is set forth in SEQ ID NO: 88. Synthetic nucleic acids comprising possible sequences conforming to the sequence set forth in SEQ ID NO: 88 were then synthesized by PCR using degenerate olignonucleotides. Exemplary sequences conforming to the consensus sequence set forth in SEQ ID NO: 88 are set forth in SEQ ID NO: 90 and SEQ ID NO: 91.

In a second process, the nucleotide sequences of nucleic acids encoding antimicrobial peptide domains of cathelicidins from a number of different species were aligned and codons encoding variable amino acids were identified. Nucleotide sequences were determined that are capable of encoding the aligned sequences. Synthetic nucleic acids comprising possible sequences conforming to the aligned sequences were then synthesized by PCR using degenerate oligonucleotides. Exemplary encoded sequences derived by this approach are set forth in SEQ ID Nos: 23-32.

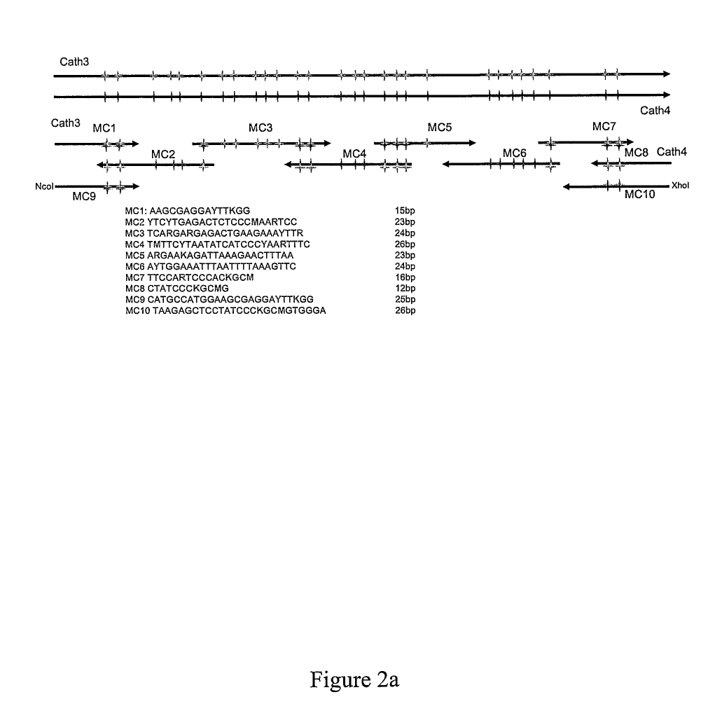

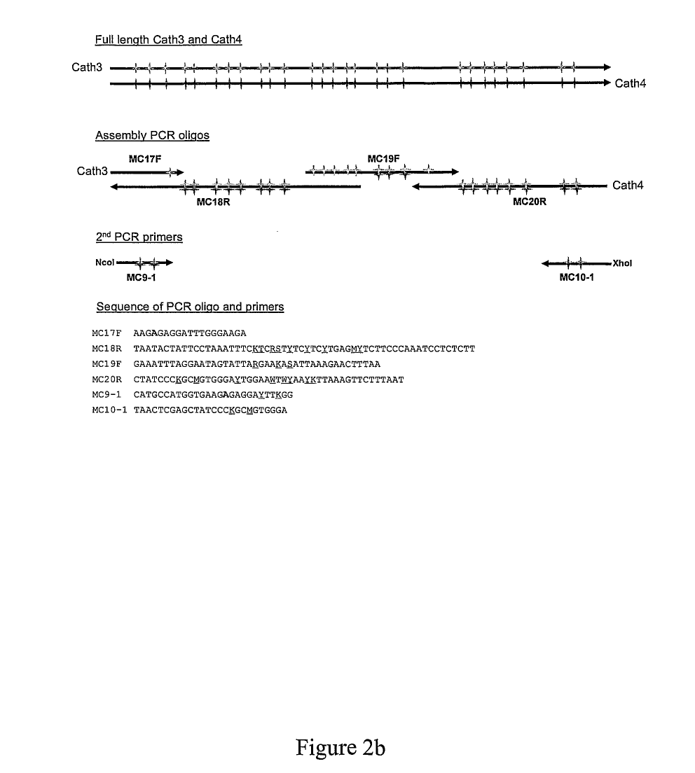

In a third process, a pool of overlapping degenerate oligonucleotides were produced that span the aligned lengths of SEQ ID NOs: 7 and 8, wherein the degenerate oligonucleotides comprise the sequences set forth in SEQ ID Nos: 113-120 (FIG. 2a). These oligonucleotides were then used in a splice overlap extension protocol to produce a single nucleic acid. Briefly, the reaction was performed using a 50 μl reaction containing 200 μM of each dNTP, 0.1 μM of each oligonucleotide, 0.5 U of Platinum Taq DNA Polymerase (Invitrogen) in a buffer containing 1.5 mM MgCl₂. The reaction was then performed with the following conditions: denaturation at 94° C. for 5 min, followed by 30 cycles at 94° C. for 30 s 20/35° C. for 30 s and 72° C. for 30 s; or denaturation at 94° C. for 5 min followed by 10 cycles at 94° C. for 30 s, 30° C. for 30 s, 72° C. for 30 s and then followed by anther 30 cycles at 94° C. for 30 s, 50° C. for 30 s and 72° C. for 30 s, and terminated by an incubation at 72° C. for 7 min. Following overlap extension, 1-2 μl of the amplification reaction was used as a template for a standard PCR in a 50 μl solution using the same reaction solution as described supra, under the same conditions as before except with primers comprising sequences set forth in SEQ ID NOs: 121 and 122. PCR cycling was conducted as follows: denaturation at 94° C. for 5 min followed by 30 cycles at 95° C. for 30 s, 55° C. for 30 s and 72° C. for 30 s, and terminated by incubation at 72° C. for 7 min.

In a third process, a pool of overlapping degenerate oligonucleotides were produced that span the aligned lengths of SEQ ID NOs: 7 and 8, wherein the degenerate oligonucleotides comprise the sequences set forth in SEQ ID Nos: 123-126 (FIG. 2b). These oligonucleotides were then used in a splice overlap extension protocol to produce a single nucleic acid. Briefly, the reaction was performed using a 50 μl reaction containing 200 μM of each dNTP, 0.1 μM of each oligonucleotide, 0.5 U of Platinum Taq DNA Polymerase (Invitrogen) in a buffer containing 1.5 mM MgCl₂. The reaction was then performed with the following conditions: denaturation at 94° C. for 5 min, followed by 30 cycles at 94° C. for 30 s 20/35° C. for 30 s and 72° C. for 30 s; or denaturation at 94° C. for 5 min followed by 10 cycles at 94° C. for 30 s, 30° C. for 30 s, 72° C. for 30 s and then followed by anther 30 cycles at 94° C. for 30 s, 50° C. for 30 s and 72° C. for 30 s, and terminated by an incubation at 72° C. for 7 min. Following overlap extension, 1-2 μl of the amplification reaction was used as a template for a standard PCR in a 50 μl solution using the same reaction solution as described supra, under the same conditions as before except with primers comprising sequences set forth in SEQ ID NOs: 127 and 128. PCR cycling was conducted as follows: denaturation at 94° C. for 5 min followed by 30 cycles at 95° C. for 30 s, 55° C. for 30 s and 72° C. for 30 s, and terminated by incubation at 72° C. for 7 min.

Amplicons produced using each of the approaches supra were directly cloned into pGEM-T easy vector (Promega), essentially according to manufacturer's instructions for sequencing to confirm their sequences. Alternatively, the amplicons were cloned into pBAD/gIIIA vector (Invitrogen) and transformed into an E. coli stain TOP10 host for expression and screening purposes.

### Sequence Analyses

To confirm that the processes described supra produced variant sequences, sequence analysis of the recombinant mutants was performed using nucleic acid from the various clones produced using fully synthesized oligonucleotides or by splice overlap extension. The sequences of peptides identified by these approaches and having antimicrobial activity are shown in SEQ ID Nos: 10-32.

### Screening of Peptides for Antimicrobial Activity

Screens were performed based on the principle that over expressing an antimicrobial peptide in bacteria will kill the host bacteria or inhibit the growth of the host bacteria. Screens were performed with recombinant nucleic acids cloned into the pBAD/gIIIA vector (Invitrogen), which contains secretion signal gene IIIA which targets proteins to the periplasmic space e.g., of E. coli. The nucleic acids were clones so as to produce an in-frame fusion between the encoded peptide and an upstream translation start site and downstream hexahistidine-encoding sequence and translation termination signal provided by the vector. As a positive control nucleic acids encoding the parental peptides were cloned into the pBAD/gIIIA vector.

Growth inhibition assays on solid LB medium were also performed essentially as described in Example 1. Briefly, primary expression was carried in several bacterial strains, i.e., E. coli strains DH5α, TOP10 and LMG194 in LB medium. Growth inhibition obviously appears in TOP10 and LMG194 stains two hours after 0.02% or 0.002% L-arabinose induction.

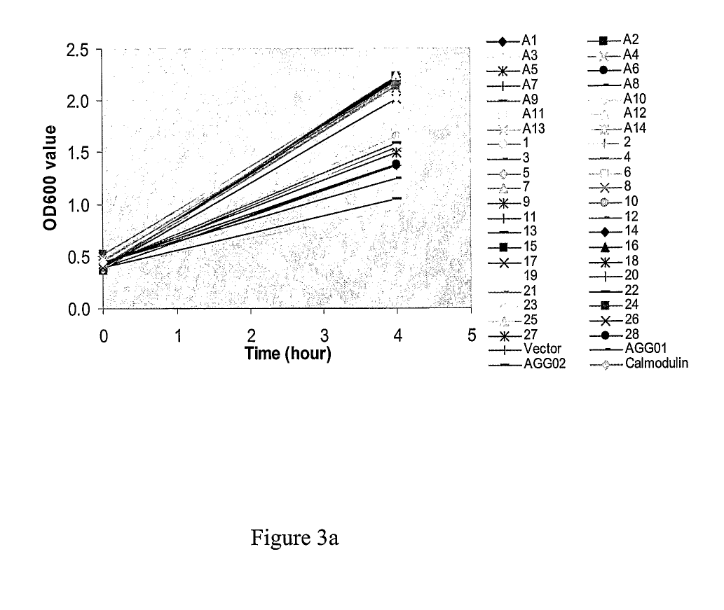

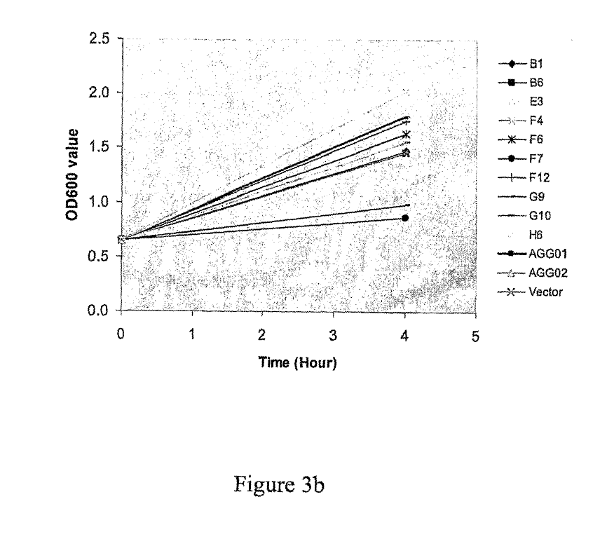

More particularly, recombinant nucleic acids were digested with NcoI and XhoI and cloned into the same sites of pBAD/gIIIA vector (FIG. 3) and then transformed into E. coli TOP10 cells. Colonies were randomly picked and sub-cultured into a 48-well plate with LB medium containing 100 μg/ml ampicillin per well at 37° C. overnight. Colonies expressing parental peptides or calmodulin were used as controls. Overnight culture was then subjected to the solid medium screen. At the same time, a 1:100 dilution of the overnight culture was also used in a liquid screen. An OD600 value was determined before induction of expression of peptides using L-arabinose and 4 hours after induction. A group of peptides capable of inhibiting bacterial growth and/or killing bacteria were then identified, in particular peptides designated A12 (SEQ ID NO: 10), 5 (SEQ ID NO: 11), 6 (SEQ ID NO: 12), 13 (SEQ ID NO: 13), A4 (SEQ ID NO: 14), A5 (SEQ ID NO: 15), A6 (SEQ ID NO: 16), A10 (SEQ ID NO: 17), A11 (SEQ ID NO: 18), 10 (SEQ ID NO: 19), 20 (SEQ ID NO: 20), 27 (SEQ ID NO: 21), 28 (SEQ ID NO: 22), B1 (SEQ ID NO: 23), B6 (SEQ ID NO: 24), E3 (SEQ ID NO: 25), F4 (SEQ ID NO: 26), F6 (SEQ ID NO: 27), F7 (SEQ ID NO: 28), F12 (SEQ ID NO: 29), G9 (SEQ ID NO: 30), G10 (SEQ ID NO: 31) and H6 (SEQ ID NO: 32) were identified as having antimicrobial activity.

The growth inhibition curves of expression of the parental peptides with control of vector expressing calmodulin is shown in FIGS. 4a and 4b. Peptides designated 5, 6, 13, A12, B6, E3, F7 and G9 inter alia inhibited bacterial growth and/or killed bacteria to a greater extent than the parental peptide comprising a sequence set forth in SEQ ID NO: 7 or SEQ ID NO: 8 (FIG. 3a, FIG. 3b).

### Example 3

### Stability of Antimicrobial Peptides in Milk

This example demonstrates the resistance of the bioactive synthetic antimicrobial peptide designated AGG01 (SEQ ID NO: 7) to proteolysis by milk proteases, thereby showing utility of the peptide in mammary glands or secretions thereof before or during or after lactation, or as a milk additive.

To determine whether or not antimicrobial peptides are bioactive in milk peptides comprising a sequence set forth in SEQ ID NO: 7 or SEQ ID NO: 8 were diluted in either 10 mM phosphate buffer or fresh or pasteurized milk to a final concentration of 200 μg/ml. Treatment groups are shown in Table 2.

TABLE 2

| | Peptide | | | | | |
|---|---|---|---|---|---|---|
| | | 2 | 3 | 4 | 5 | |
| | 1 | Fresh milk | | Pasteurized milk | | 6 |
| SEQ ID | In sodium | 37° C., 30 min | 37° C., 60 min | 37° C., 30 min | 37° C., 60 min | Milk only |
| NO: 7 | phosphate buffer, | | | | | (fresh) |
| | 4° C., 1 hour | | | | | |
| SEQ ID | In sodium | 37° C., 30 min | 37° C., 60 min | 37° C., 30 min | 37° C., 60 min | Milk only |
| NO: 8 | phosphate buffer, | | | | | (pasteurized) |
| | 4° C., 1 hour | | | | | |

Peptides having or comprising sequences set forth in any one of SEQ ID Nos: 9-83 hereof are tested similarly to demonstrate their stability in milk.

In this example, peptide AGG01 (SEQ ID NO: 7) showed antimicrobial activity in both fresh and pasteurized milk (data not shown). The antimicrobial peptide was retained in fresh milk, however was slightly reduced in pasteurized milk. Accordingly, SEQ ID NO: 7 retains bioactivity in milk, making this peptide useful for expression in a mammary gland or cell or tissue thereof and/or secretion into milk, e.g., to treat mastitis and/or to produce the peptide, e.g., as a bioreactor. Bioactivity of a peptide comprising the sequence set forth in SEQ ID NO: 8 was reduced or inhibited in the presence of fresh or pasteurized milk. Accordingly, SEQ ID NO: 8 is useful for expressing in a mammary gland or cell or tissue thereof before lactation, e.g., during pregnancy, to thereby prevent mastitis or infection by a microorganism that causes mastitis. Without being bound by theory or mode of action, sequence differences between SEQ ID NO: 7 and 8 may explain these different stabilities in milk e.g., by virtue of the presence of one or more protease recognition sequences in SEQ ID NO: 8 that are missing from SEQ ID NO: 7.

### Example 4

### Heat Resistance of Antimicrobial Peptides

This example demonstrates the heat resistance of the bioactive synthetic antimicrobial peptides designated AGG01 (SEQ ID NO: 7) and AGG02 (SEQ ID NO: 8) to heat treatment similar to that employed during pasteurisation processes for milk products, thereby showing utility of the AGG01 peptide at least as a milk additive before, during or after pasteurization occurs, and the utility of both peptides in non-dairy environments requiring heat treatments.

To determine whether or not antimicrobial peptides are bioactive following heating, e.g., following pasteurization peptides comprising a sequence set forth in SEQ ID NO: 7 or SEQ ID NO: 8 were diluted in either 10 mM phosphate buffer or fresh or pasteurized milk to a final concentration of 200 μg/ml and incubated at any of a variety of temperatures. Treatment groups are shown in Tables 3 and 4.

TABLE 3

| | Peptide | | | | | |
|---|---|---|---|---|---|---|
| | 1 | 2 | 3 | 4 | 5 | 6 |
| SEQ ID | In sodium | In pasteurised | In pasteurised | In pasteurised | In pasteurised | Milk only |
| NO: 7 | phosphate buffer, | milk, 4° C., 1 | milk, 37° C., | milk, 68° C., | milk, 71.7° C., | |
| | 4° C., 1 hour | hour | 30 min | 30 min. | 20 sec., then | |
| | | | | | 4° C. | |
| SEQ ID | In sodium | In pasteurised | In pasteurised | In pasteurised | In pasteurised | Milk only |
| NO: 8 | phosphate buffer, | milk, 4° C., 1 | milk, 37° C., | milk, 68° C., | milk, 71.7° C., | |
| | 4° C., 1 hour | hour | 30 min | 30 min. | 20 sec., then | |
| | | | | | 4° C. | |

TABLE 4

| | Peptide | | | | |
|---|---|---|---|---|---|
| | 1 | 2 | 3 | 4 | 5 |
| SEQ ID | 4° C., 1 hour | 37° C., 30 min. | 56° C., 30 min | 70° C., | Milk |
| NO: 7 | | | | 15 min. | only |
| SEQ ID | 4° C., 1 hour | 37° C., 30 min. | 56° C., 30 min | 70° C., | Milk |
| NO: 8 | | | | 15 min. | only |

Peptides having or comprising sequences set forth in any one of SEQ ID Nos: 9-83 hereof are tested similarly to demonstrate their heat stability in phosphate buffer, milk and milk products.

A peptide comprising a sequence set forth in SEQ ID NO: 7 is active in pasteurized milk, even following heat treatment (data not shown). However, the bioactivity of a peptide comprising a sequence set forth in SEQ ID NO: 8 is reduced or inhibited in pasteurized milk, as expected from the results described in Example 3. However, both peptides AGG01 and AGG02 retain their antimicrobial bioactivity in phosphate buffer following heat treatment, indicating that these peptides are heat resistant.

### Example 5

### Expression of Antimicrobial Peptides in Mammalian Cells

This example demonstrates expression and correct processing of antimicrobial peptide expressed with a prepro leader sequence in mammalian cells, and resistance of the processed peptides to proteolysis by milk proteases.

### Expression Vectors

To confirm that the antimicrobial peptides of the invention can be expressed in mammalian cells, nucleic acids encoding the antimicrobial peptides AGG01 (SEQ ID NO: 7) and AGG02 (SEQ ID NO: 8) peptides were cloned into the vector pCMV-SPORT6 (Invitrogen) in operable connection with the CMV promoter for expression in COS cells and CHO cells.

In one example, vectors designated pCMV-SPORT6-AGG01 and pCMV-SPORT6-AGG02 were produced by cloning nucleic acids encoding the antimicrobial peptides in-frame to upstream sequences encoding Macropus eugenii cathelicidin prepro sequences. The peptides expressed from pCMV-SPORT6-AGG01 and pCMV-SPORT6-AGG02 comprised the amino acid sequences set forth in SEQ ID Nos: 104 and 105.

In another example, vectors designated pCMV-SPORT6-mAGG01 and pCMV-SPORT6-mAGG02 were produced by cloning nucleic acids encoding the antimicrobial peptides in-frame to upstream sequences encoding a translation start site and a downstream translation termination signal. Peptides expressed from these vectors comprised SEQ ID No: 7 or 8.

In another example, hexahistidine-encoding sequence was placed in-frame downstream of the antimicrobial peptide and upstream of the translation termination signals in vectors designated pCMV-SPORT6-mAGG01 and pCMV-SPORT6-mAGG02, to produce the vectors designated pCMV-SPORT6-mAGG01-6×His and pCMV-SPORT6-mAGG02-6×His. Peptides expressed from these vectors were fusion proteins comprising SEQ ID No: 7 or 8 with C-terminal hexahistidine tags.

Vectors were transfected into COS cells and CHO cells according to standard procedures.

### Western Blotting

Western blotting of cell lysates was performed according to standard procedures to confirm expression of peptides in mammalian cells. Briefly, cell extracts and culture supernatants were obtained from CHO cells transfected with the vectors pCMV-SPORT6-AGG01 and pCMV-SPORT6-AGG02, resolved in 18% (w/v) SDS/polyacrylamide electrophoresis gels, transferred by electroblotting onto nitrocellulose membrane and either stained in Ponceau S or probed with polyclonal antisera. In a further example, cell extracts were incubated in bovine milk for 2 hrs prior to their preparation for electrophoresis, to determine stability in milk. Control samples comprises non-transfected CHO cell extracts and supernatants. Membranes were developed using polyclonal rabbit anti-mAGG01 serum that had been produced according to standard procedures, by immunizing rabbits with synthetic peptide (i.e., SEQ ID NO: 7). The antiserum against AGG01 (SEQ ID NO: 7) was used at a dilution of 1:4000 (v/v).

Membranes are also developed using polyclonal rabbit anti-AGG02 serum that had been produced according to standard procedures, by immunizing rabbits with synthetic peptide (i.e., SEQ ID NO: 8). The antiserum against AGG02 (SEQ ID NO: 8) is used at a dilution of 1:500 (v/v). The procedures herein are also applied to demonstrate expression of other antimicrobial peptides of the invention e.g., exemplified by SEQ ID Nos: 9-58.

In one example, expression of correctly-processed antimicrobial peptide AGG01 (SEQ ID NO: 7) was confirmed in CHO cells, by the presence of a peptide of the same size as the synthetic peptide in lysates of cells transfected with the vector pCMV-SPORT6-AGG01 and to a lesser extent in culture media, as determined in Ponceau S-stained gels and following development with anti-AGG01 serum. The peptide was also present following incubation with bovine milk for 2 hr, extending other data herein showing that the synthetic AGG01 peptide retained activity in milk (Example 3). These data suggest that the AGG01 peptide (SEQ ID NO: 7) is not processed by milk proteases and would be suitable for expressing in mammary glands during lactation or as an additive to milk and milk products. The prepro sequence and AGG01 precursor polypeptide expected to be expressed from the vector pCMV-SPORT6-AGG01 were not detectable using Ponceau S staining or anti-AGG01 serum, suggesting that the fusion polypeptide is process rapidly to a mature form by CHO cells and that the prepro sequence may be rapidly degraded.

In a further example, the CHO cell lysates and/or supernatants are also tested for their ability to inhibit growth of bacteria e.g., E. coli according to procedures described herein, to demonstrate that they retain antimicrobial activity following synthesis and processing by mammalian cells. Further purification of the peptides may be employed. For example, the peptides may be expressed as fusions with an affinity tag e.g., FLAG and isolated e.g., by nickel-NTA purification. Testing of peptides for their antimicrobial activities may be by means of performing a radial diffusion assay, e.g., as described in Example 1 for antimicrobial activity. For example, peptides are tested in a radial diffusion assay to identify those having antimicrobial activity against one or more bacteria that cause(s) mastitis, e.g., S. uberis. Alternatively, or in addition, heat stability of expressed and processed peptides is determined essentially as described in Example 4.

### Example 6

### Antibacterial Activity of Peptides Against Agents of Mastitis In Vivo

This example discloses expression vectors and methods for demonstrating efficacy of one or more peptides of the invention against one or more agents of mastitis in mammary epithelial cells in which they are expressed and, where applicable, correctly processed.

The procedures herein are applied to demonstrate expression of any antimicrobial peptide of the invention e.g., exemplified by SEQ ID Nos: 7-58 without undue experimentation.

### Expression Vectors

To confirm that the antimicrobial peptides of the invention can be expressed in mammary epithelial cells, and correctly processed by such cells to their bioactive antimicrobial form, nucleic acids encoding the antimicrobial peptides AGG01 (SEQ ID NO: 7) and AGG02 (SEQ ID NO: 8) were cloned into an expression vector e.g., pVEX, in operable connection with a bovine beta-casein promoter e.g., comprising a sequence set forth in SEQ ID NO: 92 or functional fragment thereof.

In one example, the antimicrobial peptide-encoding nucleic acids were produced by cloning nucleic acids encoding the antimicrobial peptides in-frame to upstream sequences encoding Macropus eugenii cathelicidin prepro sequences, such that the expressed peptides comprised the amino acid sequences set forth in SEQ ID NO: 104 (AGG01) and SEQ ID NO: 105 (AGG02). In this example, the AGG01 and AGG02 peptides are expressed under control of the beta-casein promoter and processed in mammary epithelial cells by virtue of cleavage of the prepro sequence from the cathelicidin proteins by endogenous mammary epithelial cell proteases.

In another example, the expression constructs of the preceding paragraph are modified to comprise nucleic acid encoding an alpha-lactalbumin signal peptide (e.g., SEQ ID NO: 101). The sequence encoding the signal peptide was placed upstream and in-frame with sequence encoding SEQ ID No: 104 or SEQ ID NO: 105. In this example, the AGG01 and AGG02 peptides are expected to be expressed as secretory proteins under control of the beta-casein promoter and signal peptide activities, and then processed in by virtue of cleavage of the prepro sequence from the cathelicidin proteins by endogenous mammary epithelial cell proteases.

In another example, the expression construct of the preceding paragraphs are further modified to comprise nucleic acid encoding a recognition sequence for enterokinase (e.g., SEQ ID NO: 112). In one example, the sequence encoding the enterokinase cleavage site is positioned in-frame between the sequence encoding the prepro sequence of M. eugenii cathelicidin and the sequence encoding AGG01 peptide (SEQ ID NO: 7) or AGG02 (SEQ ID NO: 8), to ensure correct processing in mammary epithelial cells. In another example, the sequence encoding the enterokinase cleavage site is positioned in-frame between the sequence encoding the alpha-lactalbumin signal sequence and the sequence encoding the prepro sequence of M. eugenii cathelicidin, to ensure removal of the signal peptide. In this example, the AGG01 and AGG02 peptides are in mammary epithelial cells in secretable or non-secretable form, optionally isolated, and then processed by enterokinase. Protease recognition sites that are known to be endogenous to mammary epithelial cells may promote correct processing in vivo. Protease recognition sites that are non-endogenous to mammary epithelial cells require processing in vitro.

For construction of pVEX-based expression constructs, the 554 bp early SV40 promoter of pVEX is removed by digestion using the enzymes StuI and NdeI, blunt-ended using Klenow fragment of DNA PolI, and re-circularized by self-ligation. A fragment of the β-casein promoter (SEQ ID NO: 92) is obtained by amplification of a 1.3 kb fragment there from using polymerase chain reaction (PCR) with the following primers:

(SEQ ID NO: 129)
TCTACTCGAGGATCATCTATCTGTCCCAAAG;
and
(SEQ ID NO: 130)
CTAGGATCCAATGATCTGATTTTGTGG.

The amplified fragment comprises 1230 bp of the canonical promoter plus 49 bp of the first non coding exon of the β-casein gene. The amplified β-casein promoter fragment is blunt-ended using Klenow enzyme and inserted into pVEX that has been digested using BamHI and end-filled sing Klenow enzyme. Recombinant clones are selected and nucleic acid encoding the antimicrobial peptide, optionally with in-frame upstream sequences as described in the preceding example (e.g., sequence encoding alpha-lactalbumin signal sequence and/or sequence encoding M. eugenii cathelicidin prepro sequence and/or sequence encoding a protease recognition sequence (SEQ ID NO: 112 or any one of a plasmin, MT-MSP1, Turin or urokinase recognition sequence as appropriate) is inserted into a unique HindIII site located downstream of the β-casein promoter fragment.

### Bacterial Strains and Culture Conditions

Streptococcus uberis (UT888), Streptococcus dysgalactiae subsp. dysgalactiae (UT19), and Staphylococcus aureus (UT23) isolated from dairy cows with mastitis are used. A non-pathogenic strain of Escherichia coli (DH5α; Gibco, Grand Island, N.Y., USA) is used as a negative control. Gram-positive bacteria are cultivated in Todd-Hewitt broth (THB; Becton Dickinson and Co., Franklin Lakes, N.J., USA) and E. coli is cultivated in Luria broth (LB). All bacteria are subcultured and grown on blood agar. For internalization assays, bacterial lawns are harvested, washed and resuspended at approximately 10⁷ colony-forming units per millilitre (cfu/mL) in Dulbecco's Modified Eagle's medium (DMEM, Gibco). The concentration of bacteria and strain purity are determined by standard plate count techniques.

### Fluorescent Labeling of Bacteria

Procedures for immunofluorescence staining and confocal laser microscopy (CLM) are performed essentially as described in Barker et al., Infection and Immunity 65: 1497-1504, 1997. Briefly, mastitis pathogens and E. coli DH5α kept are thawed at room temperature, plated onto blood agar, and incubated overnight at 37° C. in 5% CO₂:95% air (v/v). After incubation, the bacterial lawn is harvested with 0.5 mL of THB or LB, seeded into 9.5 mL of the same media (THB or LB), and incubated for 1 h at 37° C. with orbital shaking at 150 r.p.m. After incubation, bacterial suspensions are washed three times by centrifugation (20 800 g for 3 min at 4° C.) with phosphate-buffered saline (PBS) (pH 7.4) and fluorescently labeled (LIVE/DEAD BacLight Bacterial Viability Kits L-7007; Molecular Probes, Eugene, Oreg., USA) following manufacturer's instructions.

### Mammary Epithelial Cell Culture

A bovine mammary epithelial cell line (MAC-T) (Huynh et al., Exp. Cell. Res., 197: 191-199, 1991) is used for infection studies. Cells are transfected with a nucleic acid encoding an antibacterial peptide of the invention e.g., any one of SEQ ID Nos: 7-58. Transfected MAC-T cells are grown in 24-well cell culture plates (Corning Inc., Corning, N.Y., USA) or 8-well slides (Lab-Tek II; Nalge Nunc International Corp., Rochester, N.Y., USA) at 37° C. in 5% CO2:95% air (v/v) using cell growth media described previously (Almeida et al., J. Vet. Med., 45: 385-392, 1996). Mammary epithelial cell viability is monitored by trypan blue dye exclusion.

### Internalization and Intracellular Bactericidal Assay

Fluorescent-labeled or untreated mastitis pathogens or E. coli DH5α are co-cultured with MAC-T cells in DMEM. Following incubation [2 h at 37° C. in 5% CO₂:95% air (v/v)], monolayers are washed three times with PBS (pH 7.4).

As a control, co-cultures are incubated with non-transfected cells or non-transfected cells in DMEM containing gentamicin (100 μg/mL; Sigma Chemical Co., St Louis, Mo., USA) and penicillin (100 IU/mL, Sigma). Penicillin and gentamicin do not penetrate into mammary epithelial cells and are used in the standard internalization protocol to eliminate bacteria that are outside of mammary epithelial cells and allow discrimination between intracellular and extracellular micro-organisms. After removing media containing antibiotics, MAC-T cell monolayers are washed and lysed. MAC-T cell lysates are 10-fold serially diluted, plated in triplicate on blood agar, and incubated overnight. Intracellular survival is evaluated by determining the number of cfu/mL in MAC-T cell lysates. For fluorescent assays, after incubation bacteria, slides are washed with PBS (pH 7.4) and mounted. Cover slips are then sealed onto slides with nail polish and kept at 4° C. until visualization by CLM (Leica TCS SP2; Leica Microsystems, Heidelberg, Germany). Internalization and intracellular survival assays are performed in triplicate three times.

### Image Analysis

Red/green images are collected and overlaid using Leica Lite software (Leica Microsystems, Heidelberg, Germany).

### Results

Peptides capable of killing bacteria or preventing growth of bacteria within a mammary cell, e.g., as determined by reducing c.f.u from lysed MAC-T cells compared to non-transfected cells and/or that reduce the number of live bacteria and/or increase the number of dead bacteria within MAC-T cells are selected as peptides suitable for treatment of mastitis.

### Example 7

### Production of Transgenic Cows

This example discloses expression vectors and methods for producing transgenic cattle, especially cows, expressing the antimicrobial peptide(s) of the invention, and the efficacy of the expressed peptides in protecting animals against mastitis.

It is to be understood that the procedures herein are applied to demonstrate expression and efficacy of any antimicrobial peptide of the invention e.g., exemplified by SEQ ID Nos: 7-58 without undue experimentation.

### Expression Vectors

Any of the expression vectors described in the preceding example are employed herein, especially those in a pVEX backbone or other at-recognized vector suitable for transfection of somatic cells and their subsequent fusion with enucleated oocytes to produce transgenic cattle.

a) Preparation of pVEβcashAMP

An expression construct pVEβcashAMP is produced essentially in accordance with the description in Example 6, employing a pVEX backbone. The vector is modified to express gene constructs encoding the antimicrobial fusion proteins as described in Example 6.

### Transfection of Somatic Cells

Expression vectors based on pVEβcashAMP are used for transfecting a primary culture of somatic cells, using calcium phosphate or liposome method. Fetal calf fibroblasts are generally transfected. Transfected cells are then selected using geneticin, and following a period of 2 to 8 weeks, the cells that are resistant to geneticin are isolated for use as donor cells to obtain transgenic clones. Transfected selected cells are analyzed by PCR to confirm presence of the expression construct to ensure the appropriate nuclei are transferred to generate transgenic embryos.

### Oocyte Enucleation and Metaphase Nuclear Transfer in Mature Enucleated Oocytes

Bovine oocytes are aspirated from slaughterhouse ovaries and matured in TCM-199+5% FCS at 39° C. for 24 hours. The maturation medium is equilibrated with CO₂ for at least 2 hours prior to use. Mature oocytes are denuded by vortexing for 2 minutes in warm TL-HEPES with 1 mg/ml bovine testis hyaluronidase.

### Nuclear Transfer

Oocytes are treated with roscovitine to suspend meiosis. Oocytes are mechanically enucleated using a Narishige hydraulic micromanipulators and Nikon Diaphot microscopy. Enucleation is performed with a 20 μm beveled and sharpened pipette. Oocytes are stained with 5 μg/ml bisbenzimidine (Hoechst 33342) dye for 20 minutes. Metaphases are enucleated by visualization of the stained chromosomes under ultraviolet light. Metaphase chromosomes are assessed after aspiration inside the pipette. A transgenic somatic cell produced as described supra is then transferred into the perivitelline space and tightly opposed to the enucleated oocyte.

### Fusion

A transgenic somatic cell and an enucleated oocyte are manually aligned in the fusion chamber so that the membranes to be fused are parallel to the electrodes. Fusion is performed using one electrical pulse of 180 volts/cm for 15 μs (BTX Electro Cell Manipulator 200) and monitored with a BTX Optimizer-Graphic Pulse Analyzer. The chamber for pulsing embryos consists of two 0.5 mm stainless steel wire electrodes mounted 0.5 mm apart on glass microscope slide. Presumptive zygotes are monitored for fusion, lysis, and fragmentation.

### Transfer to Recipient Cows

Zygotes are evaluated at 48 hours after fertilization for cleavage and after 7 to 9 days for development to morulae or blastocysts. Generally, two blastocysts are transferred non-surgically per recipient cow, and pregnancies determined at 30-35 days by ultrasonography.

The implanted cows are allowed to normally pass the pregnancy up to a natural delivery. Newborn calves are fed with Ig rich colostrum during the first 48 hours, and then synthetic, later natural foods are used.

Following sufficient time for transgenic animals to reach maturity, animals are bred to produce female transgenic cows.

### Example 8

### Production of Transgenic Goats

This example discloses expression vectors and methods for producing transgenic goats expressing the antimicrobial peptide(s) of the invention, and the efficacy of the expressed peptides in protecting animals against mastitis.

It is to be understood that the procedures herein are applied to demonstrate expression and efficacy of any antimicrobial peptide of the invention e.g., exemplified by SEQ ID Nos: 7-58 without undue experimentation.

### Expression Constructs

A 2.0-kb promoter fragment from bovine alpha-lactalbumin (α-LA) gene is generated by PCR amplification using a genomic DNA from high milk-producing Holstein cow as the template. This PCR product containing entire α-LA promoter and 19-aa leader sequence are then subsequently inserted into the pCR3 vector (Invitrogen, San Diego, Calif.). Nucleic acid encoding an antimicrobial peptide (any one of SEQ ID Nos: 7-58 or a fusion construct as described in Example 6 or 7 lacking the introduced alpha-lactalbumin signal peptide-encoding sequence) is cloned into the pCR3 vector in-frame with the leader sequence to produce the vector pCR3-α-LA-AMP.

### Transgenic Animal Production

For transgenic goat production, pronuclear stage embryos are flushed from a donor goat's oviduct at the one and half day after insemination. The collected embryos are then rinsed with sterile phosphate buffered saline and placed under a phase contrast microscope for microinjection of the expression construct. An expression construct is then microinjected into the male pronucleus of the embryo. After a transient in vitro culture, healthy microinjected embryos are then transferred into recipient oviducts for development into a transgenic goat.

Following birth, ear punctures are taken from newborn goats and genomic DNA isolated there from. PCR is then performed suing the genomic DNA as a template to determine whether or not each newborn goat comprises the expression construct. Goats comprising the expression construct are then bred with wild-type goats to produce female goats, which are also screened to select those comprising the expression construct.

### Example 9

### Preparation of a Monoclonal Antibody that Recognizes an Antimicrobial Peptide

This example discloses a procedure for preparing monoclonal antibodies that specifically recognize antimicrobial peptides of the invention for diagnostic purposes. The procedure is applied to any peptide disclosed herein as SEQ ID Nos: 7-83.

A monoclonal antibody that specifically binds to an antimicrobial peptide comprising a sequence set forth in any one of SEQ ID Nos: 7-83 is produced using methods known in the art. Briefly, the peptide antigen is synthesized essentially using the methods described in Bodanszky, M. (1984) Principles of Peptide Synthesis, Springer-Verlag, Heidelberg and Bodanszky, M. & Bodanszky, A. (1984) The Practice of Peptide Synthesis, Springer-Verlag, Heidelberg. Peptides are purified using HPLC and purity assessed by amino acid analysis.

Female BalB/c mice are immunized with a purified form of the peptide. Initially mice are sensitized by intraperitoneal injection of Hunter's Titermax adjuvant (CytRx Corp., Norcross, Ga.,). Three boosts of the peptide are administered at 2, 5.5 and 6.5 months post initial sensitization. The first of these boosts is a subcutaneous injection while the remaining are administered by intraperitoneal injection. The final boost is administered 3 days prior to fusion.

The splenocytes of one of the immunized BALB/c mice is fused to X63-Ag8.653 mouse myeloma cells using PEG 1500. Following exposure to the PEG 1500 cells are incubated at 37° C. for 1 hour in heat inactivated fetal bovine serum. Fused cells are then transferred to RPMI 1640 medium and incubated overnight at 37° C. with 10% CO₂. The following day, cells are plated using RPMI 1640 media that has been supplemented with macrophage culture supernatants.

Two weeks after fusion, hybridoma cells are screened for antibody production by solid phase ELISA assay. Standard microtitre plates are coated with the peptide antigen in a carbonate based buffer. Plates are then blocked with BSA, washed and then the test samples (i.e. supernatant from the fused cells) is added, in addition to control samples, (i.e. supernatant from an unfused cell). Antigen-antibody binding is detected by incubating the plates with goat-anti-mouse HRP conjugate (Jackson ImmunoResearch Laboratories) and ABTS peroxidase substrate system (Vector Laboratories, Burlingame, Calif. 94010, USA). Absorbance is read on an automatic plate reader at a wavelength of 405 nm.

Any colonies that are identified as positive by these screens continue to be grown and screened for several further weeks. Stable colonies are then isolated and stored at −80° C.

Positive stable hybridomas are then cloned by growing in culture for a short period of time and diluting the cells to a final concentration of 0.1 cells/well of a 96 well tissue culture plate. These clones are then screened using the previously described assay. This procedure is then repeated in order to ensure the purity of the clone.

Four different dilutions, 5 cells/well, 2 cells/well, 1 cell/well, 0.5 cells/well of the primary clone are prepared in 96-well microtiter plates to start the secondary cloning. Cells are diluted in IMDM tissue culture media containing the following additives: 20% fetal bovine serum (FBS), 2 mM L-glutamine, 100 units/ml of penicillin, 100 μg/ml of streptomycin, 1% GMS-S, 0.075% NaHCO3. To determine clones that secrete anti-antimicrobial peptide antibody, supernatants from individual wells of the 0.2 cells/well microtiter plate are withdrawn after two weeks of growth and tested for the presence of antibody by ELISA assay as described above.

All positive clones are then adapted and expanded in RPMI media containing the following additives: 10% FBS, 2 mM L-glutamine, 100 units/ml of penicillin, 100 μg/ml of streptomycin, 1% GMS-S, 0.075% NaHCO3, and 0.013 mg/ml of oxaloacetic acid. A specific antibody is purified by Protein A affinity chromatography from the supernatant of cell culture.

The titers of the antibodies produced using this method are determined using the Easy Titer kit available from Pierce (Rockford, Ill., USA). This kit utilizes beads that specifically bind mouse antibodies, and following binding of such an antibody these beads aggregate and no longer absorb light to the same degree as non-associated beads. Accordingly, the amount of an antibody in the supernatant of a hybridoma is assessed by comparing the OD measurement obtained from this sample to the amount detected in a standard, such as for example mouse IgG.

The specificity of the monoclonal antibody is then determined by Western blotting according to standard procedures.

### Example 10

### Immunoblot Analysis of Milk from Transgenic Cows and Goats

This example discloses diagnostic procedures for determining expression of antimicrobial peptides in transgenic animals e.g., cattle and goats such as for the selection of breeding stocks expressing the antimicrobial peptides at particular levels.

Milk is collected from lactating females produced in Examples 7 and 8 essentially as described in Simons et al., Nature 328: 530-532, 1987) and resolved using SDS-polyacrylamide gel electrophoresis (SDS-PAGE) essentially as described in Cheng et al., Human Gene Therapy 9: 1995-2003, 1998). Proteins are electro-transferred from the gel to a PVDF membrane. The blots are then probed with the monoclonal antibody described in Example 7 and washed with phosphate-buffered saline containing 0.1% Tween-20 (PBS-T). Blots are then incubated with an anti-mouse secondary antibody conjugate to horseradish peroxidase (HRP). Blots are then developed with the chemiluminescent ECL™ detection system (Amersham, UK) and exposure to x-ray film. Band intensities were compared by densitometry.

Transgenic goats and cows secreting an antimicrobial peptide into their milk are then selected.

### Example 11

### Antimicrobial Activity of Milk from Transgenic Animals

This example discloses diagnostic procedures for determining activity of antimicrobial peptides expressed in milk of in transgenic animals e.g., cattle and goats such as for the selection of breeding stocks.

### Milk Collection

Milk is either collected from goats or cows described in Example 7 or 8 using an automated sampling device during the normal process of milking or collected by hand in an aseptic manner. All samples are centrifuged at 800 g for 15 min at 4° C. and the supernatant collected and tested immediately or frozen at −20° C. until use.

### Antimicrobial Assays

### 1. Radial Diffusion Assay

Spot-on-lawn assays are then performed to compare the antimicrobial activity of milk from transgenic cows or goats with that of synthetic antimicrobial peptides. Samples are assayed in triplicate and a dilution series of synthetic peptide is included in the assay. S. uberis or S. aureus or E. coli in log-phase growth containing are added to a Petri dish containing appropriate growth medium. Dishes are air dried for 30-60 min before 10 μl of a dilutions series (from undiluted to 1:32) of test milk samples or synthetic peptide standards, both diluted in skim milk (Difco Laboratories), are added to the dishes. After an additional 30-min drying time, dishes are inverted and incubated at 37° C. in air overnight.

Optical density of cleared zones is determined with a ChemiDoc XRS with Quantity One software (Bio-Rad Laboratories). The antimicrobial activity of milk from transgenic cows or goats is quantified by comparing optical densities of cleared zones with optical densities produced by synthetic peptides in a dilution series. In this manner, a level of bioactivity of peptide expressed in cow's milk relative to synthetic peptide is determined.

### 2. Mastitis Challenge Assays

Forty-eight hours before initiating bacterial challenges, health of the animals as described in Example 7 or 8 is assessed by differential leukocyte and milk somatic cell counts to verify that they are within normal ranges. Animals that appear not to be suffering from mastitis are then infected with a mastitis causing bacterium by infusing 2 ml S. uberis and/or S. aureus and/or E. coli via the streak canal after morning milking. One animal is infused with 2 ml of sterile PBS. The cows and goats are closely monitored, initially at 6-h intervals, and then every 12 h for 48 h. Body temperature, blood and milk samples are taken at 12-h intervals or more frequently throughout the study. Milk samples (20 l) are plated on suitable growth medium depending on the bacteria infused into the goat or cow and incubated at 37° C. for 18 to 24 h. Once an infection was confirmed by the presence of viable S. uberis and/or S. aureus and/or E. coli in two consecutive milk samples, the animal is treated with antibiotics for five consecutive milkings. Milk somatic cells and bacteria are monitored weekly thereafter to assure that infections are eliminated.

For enumeration of somatic cells, milk samples were heated to 60° C. for 15 min, cooled to 40° C. and cells counted on a Fossomatic 90 (Foss Electric). The device is calibrated quarterly with bovine milk somatic cell standards (Dairy Quality Control Institute Services). Samples are counted in duplicate. Cows and goats that do not show significant increases in somatic cell counts following infusion of an infectious bacteria are considered resistant to that bacteria and mastitis.

To determine whether or not transgenic animals are resistant to mastitis, each animal is infused a plurality of times with an infectious bacteria and the number of infections resulting determined. Animals that do not become infected or rarely become infected are considered resistant to mastitis causing bacteria.

### Example 12

### Synergistic Effect of Antimicrobial Peptides and Antibiotics

This example discloses synergism between synthetic antimicrobial peptides of the invention and known antibiotics. The procedure is applied to any peptide disclosed herein as SEQ ID Nos: 7-83.

### Methods

To determine whether or not antimicrobial peptides as described herein act in a synergistic manner with antibiotic compounds, a microbroth dilution method was performed against E. coli DH5α performed according to the guidelines of the National Committee for Clinical Laboratory Standards.

### Results

As shown in Table 5, a peptide comprising a sequence set forth in SEQ ID NO: 7 or 8 reduces the MIC of tetracycline or chloramphenicol indicating that these peptides act synergistically with tetracycline or chloramphenicol.

TABLE 5

| | MIC (μg/ml) | | |
|---|---|---|---|
| Combination | — | SEQ ID NO: 7 | SEQ ID NO: 8 |
| Ampicillin | 2 | 2 | 4 |
| Chloramphenicol | 4 | 1 | 2 |
| Tetracycline | 2 | 0.25 | 0.5 |

### Example 13

### Efficacy of Antimicrobial Peptides in the Treatment of Bovine Respiratory Disease and/or Swine Respiratory Disease

This example discloses the efficacy of antimicrobial peptide of the invention against known agents of BRD and/or SRD, as determined by the MIC value. The procedure is applied to any peptide disclosed herein as SEQ ID Nos: 7-83.

TABLE 6
Peptide activity against BRD/SRD isolates

| | |
|---|---|
| Microorganism | MIC (μg/ml) |

| [No. strains tested] | SEQ ID NO: 7 | SEQ ID NO: 8 |
|---|---|---|
| E. coli [9] | 1-8 | 8-32 |
| P. haemolytica [4] | 1-16 | 4-64 |
| P. multicoda [4] | 16 | 8-32 |
| B. bronchiseptica [2] | 1-2 | 32 |
| H. somnus [3] | 16 | 32 |
| A. pleuropneumoniae [3] | 8 | 16 |
| S. suis [3] | 16-32 | 4-8 |
| S. choleraesuis [3] | 4-8 | >64 |

The data presented in Table 6 indicate utility of peptide AGG01 in particular against one or more agents of BRD/SRD, with especially strong activity against isolates of E. coli, B. bronchiseptica and S. choleraesuis and possibly also against P. haemolytica, Intermediate activity was observed against A. pleuropneumoniae and S. choleraesuis, and weaker activity against S. suis and H. somnus.

### Example 14

### Efficacy of Antimicrobial Peptides in the Treatment of Otitis Externa

This example discloses the efficacy of antimicrobial peptide of the invention against known agents of otitis externa e.g., in canines, as determined by the MIC value. The procedure is applied to any peptide disclosed herein as SEQ ID Nos: 7-83.

TABLE 7
Peptide activity against otitis externa isolates

| | |
|---|---|
| Microorganism | MIC (μg/ml) |

| [No. strains tested] | SEQ ID NO: 7 | SEQ ID NO: 8 |
|---|---|---|
| S. aureus [1] | 2 | 4 |
| S. schleiferi [5] | 2-4 | 2-4 |
| S. epidermis [1] | 1 | 2 |
| S. pseudointermedin [3] | 1-2 | 1 |
| Pseudomonas aeruginosa [5] | 2-4 | 8-32 |

The data presented in Table 7 indicate utility of peptides AGG01 (SEQ ID NO: 7) and AGG02 (SEQ ID NO: 8) against one or more agents of otitis externa, with especially strong activity against isolates of S. aureus, S. schleiferi, S. epidermis and S. pseudointermedin. Strong antimicrobial activity was observed using SEQ ID NO: 7 against Pseudomonas aeruginosa, with slightly weaker activity against this pathogen using SEQ ID NO: 8.

1. A peptide or an analog or derivative thereof, said peptide, analog or derivative having antimicrobial activity at least against Streptococcus uberis.

2. The peptide or analog or derivative according to claim 1 having antimicrobial activity against S. uberis and a further organism selected from the group consisting of S. suis, S. agalactiae, P. aeruginosa, E. coli, S. aureus, S. schleifen subsp. coagulans, S. schleiferi, S. epidermis, S. pseudointermedin, Mannheimia haemolytica (P. haemolytica), P. multocida, A. pleuropneumoniae (APP), H. somnus, Salmonella choleraesuis and B. bronchiseptica and any combination thereof.

3. The peptide or analog or derivative according to claim 1 having low activity against one or more lactobacilli.

4. The peptide or analog or derivative thereof according to claim 1, wherein said peptide or analog or derivative thereof retains antimicrobial activity at salt concentrations normally found in a milk product and/or retains antimicrobial activity in milk or other dairy product.

5. The peptide or analog or derivative according to claim 1, wherein the antimicrobial activity of said antimicrobial peptide or analog or derivative thereof is reduced or antagonized or partially or completely inhibited when contacted with milk.

6. The peptide or analog or derivative according to claim 1, wherein said peptide or analog or derivative thereof has antimicrobial activity following heating to a temperature at which milk or a milk product or other dairy product is pasteurized.

7. The peptide or analog or derivative according to claim 6, wherein said peptide or analog or derivative thereof has antimicrobial activity in milk following heating to a temperature at which milk or a milk product or other dairy product is pasteurized.

8. The peptide or analog or derivative according to claim 1, wherein the antimicrobial activity of said peptide or analog or derivative thereof is completely or partially reduced or inhibited in milk following heating to a temperature at which milk or a milk product or other dairy product is pasteurized.

9. The peptide or analog or derivative thereof according to claim 1, wherein said analog is a retro-peptide analog.

10. The peptide or analog or derivative thereof according to claim 1, wherein said analog is a retro-inverted peptide analog.

11. A composition comprising one or more antimicrobial peptides and/or analogs and/or derivatives according to claim 1 and a suitable carrier or excipient.

12. An expression construct comprising nucleic acid encoding the peptide and/or analog and/or derivative thereof according to claim 1 operably linked to a promoter that confers expression on said nucleic acid in a mammary gland or cell or tissue thereof.

13-19. (canceled)

20. An expression vector comprising the expression construct according to claim 12.

21. A virus particle comprising the expression construct according to claim 12.

22. A genetically-modified non-human mammal comprising the expression construct according to claim 12 or a zygote or an embryo or an offspring or reproductive material thereof comprising said expression construct.

23-25. (canceled)

26. A method for treating or preventing a disease or condition selected from the group consisting of mastitis, respiratory disease, clostridial intestinal disease and otitis externa in human or a non-human mammal, said method comprising administering to a mammal in need of treatment or prophylaxis a peptide and/or analog and/or derivative according to claim 1 or a pharmaceutical composition comprising one or more of said peptide, analog or derivative or an expression construct expressing said peptide, analog or derivative.

27. A method for treating or preventing mastitis in a mammal, said method comprising expressing in a mammary gland of a mammal in need of treatment or prophylaxis or a cell or tissue thereof a peptide and/or analog and/or derivative according to claim 1.

28-29. (canceled)

30. A process for improving milk production in a non-human mammal, said process comprising performing the method according to claim 27 in a non-human mammal to thereby prevent or treat mastitis in a non-human mammal thereby improving milk production in the genetically-modified non-human mammal.

31. A process for improving production of a recombinant polypeptide in milk of a genetically-modified non-human mammal, said process comprising performing the method according to claim 27 in a non-human mammal that is genetically modified to thereby secrete the recombinant polypeptide into milk produced from its mammary gland(s), thereby enhancing production of milk comprising the recombinant polypeptide by the genetically-modified non-human mammal.

32. A method of producing a dairy product comprising lactobacilli or for which lactobacilli are used in production said method comprising providing an antimicrobial peptide, analog or derivative according to claim 1 to the dairy product for a time and under conditions sufficient to prevent contamination.

33. A method for producing the antimicrobial peptide and/or analog and/or derivative according to claim 1, said method comprising:
(i) producing or obtaining a genetically-modified non-human mammal capable of expressing the peptide, analog or derivative; and
(ii) maintaining the genetically-modified non-human mammal for a time and under conditions sufficient for the antimicrobial peptide and/or analog and/or derivative to be expressed,
thereby producing the antimicrobial peptide and/or analog and/or derivative.

34-52. (canceled)
